(19) United States
(12) Patent Application Publication
(10) Pub. No.: US 2021/0155930 A1
(43) Pub. Date: May 27, 2021

(54) MICRORNA TARGETING AGENT FOR TREATMENT OF HEART DISEASE

(71) Applicant: UNITED KINGDOM RESEARCH AND INNOVATION, Swindon (GB)

(72) Inventors: Jaya KRISHNAN, Kronberg im Taunus (DE); Corinne BISCHOF, Münsingen (CH)

(73) Assignee: UNITED KINGDOM RESEARCH AND INNOVATION, Swindon (GB)

(21) Appl. No.: 17/256,206

(22) PCT Filed: Jun. 28, 2019
(86) PCT No.: PCT/EP2019/067497

§ 371 Date: Dec. 27, 2020

(30) Foreign Application Priority Data
Jun. 28, 2018 (EP) 18180592.0

(51) Int. Cl. C12N 15/113; A61P 9/00

(52) U.S. Cl. CPC C12N 15/113; C12N 2310/3231; C12N 2310/113; A61P 9/00

(57) ABSTRACT

The present invention relates to an oligonucleic acid agent directed against the microRNA miR27b-5p for use in a method of treatment or prevention of heart disease.

The present invention relates to the treatment and prevention of heart disease by administering oligonucleic acid agents that modulate the activity or expression of microRNAs. More precisely, the invention provides methods for treating or preventing heart disease by inhibiting the expression and/or activity of the microRNA miR27b-5p.

### BACKGROUND OF THE INVENTION

The mechanism through which stress signaling promotes endoreplication, and thus the replication of DNA during S phase of the cell cycle without subsequent completion of mitosis and/or cytokinesis, to raise the cell size threshold is unknown. While the analysis of cell cycle regulation, growth signaling pathways, and transcriptional and translational networks has provided insight into upstream control networks, the more fundamental question of the underlying energetics in which the aforementioned regulators can function remains largely unaddressed. The energetic state of a cell not only determines ploidy, which is by definition the number of sets of chromosomes in a cell, but also cell size and function to establish an environment permissive for specific outputs that are co-opted by signaling cascades and transcriptional regulators to implement specific growth or functional endpoints. Emerging evidence links deregulated ADP:ATP homeostasis to the development of human hypertrophic cardiomyopathy (HCM) and aortic stenosis (AS). HCM and AS epitomize the cardiac overgrowth phenotype and are characterized by endoreplication, resulting in polyploidy and multinucleation (cells with two or more nuclei), and pathologic cardiomyocyte growth in an environment of depressed mitochondrial ATP synthesis. It is paradoxical that energetically deficient cardiomyocytes, exhibiting a reduced capacity to generate energy and cofactors though mitochondrial oxidation, are able to drive anabolic processes to support cardiomyocyte hypertrophy. In accord, mitochondrial myopathies are predominantly characterized by cardiac overgrowth.

Given that cardiac hypertrophy is metabolically characterized by depressed mitochondrial ATP synthesis we commenced on systematically profiling the expression of all subunits of the ATP synthase complex in patient biopsies of human HCM and AS. We identified ATP5A1 (encoding for the a-subunit of the ATP synthase catalytic F₁ complex which forms together with the p-subunit, the catalytic domain through which the central stalk rotates to regenerate ATP from ADP and inorganic phosphate) as downregulated in HCM and AS biopsies. Mechanistically, we demonstrate that ATP5A1 repression is mediated by the stress dependent-regulated hypoxia-inducible factor (HIF)1a driven induction of MIR27B. Inhibition of ATP5A1 suppresses mitochondrial ATP synthase activity, leading to accumulation of intra-mitochondrial ADP that is channeled and serves as a cofactor for MTHFD1L, a mitochondria localized rate-limiting enzyme of the 1-carbon pathway regulating formate and purine biosynthesis. Activation of MTHFD1L by ADP in cardiomyocytes accelerates de novo nucleotide synthesis and drives cardiomyocyte endoreplication and cell hypertrophy through parallel activation of AMPK-driven E2F. Consequently, MIR27B inactivation in vitro and in mice attenuates pre-existing heart failure in response to surgery-mediated aortic stenosis (transaortic constriction (TAC)), while cardiac-specific MIR27B expression results in spontaneous cardiac overgrowth. Analyses of the downstream components, ATP5A1 and MTHFD1L in mice, further support a central role for the HIF1α-MIR27B-ATP5A1-MTHFD1L axis in cardiac polyploidization and cell size control. In accord, activation of this axis correlates with human pathologic cardiac hypertrophy. These findings reveal a stress-dependent pathway connecting deregulated mitochondrial ATP homeostasis with pathophysiologic endoreplication in cardiomyocytes, resulting in multinucleation and morphologic growth.

As the inventors could identify cardiometabolic endoreplication as a hitherto unknown mechanism dictating pathologic growth progression in the energetically deficient myocardium through miR27b-5p overexpression, there is a need in the art for treatment methods that can inhibit the expression of miR27b-5p for prevention of pathological growth through endoreplication and de novo nucleotide biosynthesis.

Based on the above-mentioned state of the art, the objective of the present invention is to provide means and methods for treatment of heart disease, particularly cardiomyopathy. This objective is attained by the subject matter of the claims of the present specification.

### DESCRIPTION

The present invention is based upon the discovery that the inhibition of miRNA miR27b-5p through administration of an oligonucleic acid agent (SEQ ID NO 001) enables the modification of pathological growth of the myocardium via regulators such as ATP synthase, which drives mitochondrial ADP build-up and its redirection to methylenetetrahydrofolate dehydrogenase 1L (MTHFD1L) and de novo nucleotide biosynthesis.

The term miRNA in the context of the present invention relates to pri-, pre- and mature miRNA.

There are two mature subspecies of the stem-loop sequence of miRNA27b, namely miRNA27b-3p and miRNA27b-5p.

“Capable of forming a hybrid” in the context of the present invention relates to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

The term oligonucleic acid agent in the context of the present specification refers to an oligonucleotide capable of specifically binding to and leading to a significant reduction of the physiological role of miR27b-5p. Examples of oligonucleic acid agents of the present invention are antisense oligomers made of DNA, DNA having phosphorothioate modified linkages in their backbone, ribonucleotide oligomers, RNA comprising bridged or locked nucleotides, particularly wherein the ribose ring is connected by a methylene bridge between the 2′-O and 4′-C atoms, RNA having phosphorothioate modified linkages in their backbone or any mixture of deoxyribonucleotide and ribonucleotide bases as an oligomer.

The term antisense oligonucleotide or oligonucleotide agent in the context of the present specification refers to any oligonucleotide capable of specifically binding to and leading to a significant reduction of the physiological role of miR27b-5p. Examples of antisense oligonucleotides of the present invention are antisense oligomers made of DNA, DNA having phosphorothioate modified linkages in their backbone, ribonucleotide oligomers, RNA comprising bridged or locked nucleotides, particularly wherein the ribose ring is connected by a methylene bridge between the 2′-O and 4′-C atoms, RNA having phosphorothioate modified linkages in their backbone or any mixture of deoxyribonucleotide and ribonucleotide bases as an oligomer.

The terms oligonucleic acid agent and antisense oligonucleotide or oligonucleotide agent are used interchangeably in the present specification.

In certain embodiments, the antisense oligonucleotide of the invention comprises analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The antisense sequence may be composed partially of any of the above analogues of nucleic acids, with the rest of the nucleotides being “native” ribonucleotides occurring in nature, or may be mixtures of different analogues, or may be entirely composed of one kind of analogue.

The term gapmer is used in its meaning known in the field of molecular biology and refers to an antisense oligonucleotide complementary to its target sequence, that comprises a central block of a deoxyribonucleotide oligomer flanked by short ribonucleotide oligomers. The flanking ribonucleotide oligomers consist of nuclease and protease resistant ribonucleotides.

In certain embodiments, the nuclease and protease resistant ribonucleotides comprise 2′-O modified ribonucleotides, in particular bridged nucleic acids with a bridge between the 2′-O and 4′-C of the ribose moiety.

“Nucleotides” in the context of the present invention are nucleic acid or nucleic acid analogue building blocks, oligomers of which are capable of forming selective hybrids with miRNA oligomers on the basis of base pairing. The term nucleotides in this context includes the classic ribonucleotide building blocks adenosine, guanosine, uridine (and ribosylthymin), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed of any of the above nucleotides, or mixtures thereof.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear.

Unless otherwise stated, sequence identity values provided herein refer to the value obtained using the BLAST suite of programs (Altschul et al., J. Mol. Biol. 215:403-410 (1990)) using the above identified default parameters for protein and nucleic acid comparison, respectively.

### DETAILED DESCRIPTION OF THE INVENTION

A first aspect of the invention provides an oligonucleic acid agent directed against the microRNA miR27b-5p for use in a method of treatment or prevention of heart disease.

In other words, the oligonucleic acid agent of the invention is directed against, or capable of forming a hybrid with, the miRNA miR27b-5p (SEQ ID NO 002).

In certain embodiments, the oligonucleic acid agent is directed against miR27b-5p miRNA in its mature form.

In certain embodiments, the oligonucleic acid agent is capable of reducing miR27b-5p miRNA levels in a cell by an amount (expressed in percentage) of at least 10-20%, at least 30-40%, at least 50-60%, at least 70-80%, at least 90-98%, or at least 99% when the oligonucleic acid agent is introduced into a mammalian cell.

In certain embodiments, the oligonucleic acid agent for use in a method of treatment or prevention of heart disease comprises, or essentially consists of, the sequence ACC AAT CAG CTA AGC T (SEQ ID NO 001).

The oligo nucleic acid agents of the invention are defined by their sequence; however, the skilled person understands that by exchanging one or two positions, particularly while increasing binding to the mRNA through introduction of nucleotide analogues, sufficient specificity of binding may be attained to achieve the inventive effect.

In certain embodiments, the oligonucleic acid agent comprises a sequence hybridizing to miR27b-5p. The agent sequence is at least 95% identical, particularly 96%, 97%, 98%, 99% or 100% identical to SEQ ID 001. In certain embodiments, the hybridizing sequence comprises deoxynucleotides, phosphothioate deoxynucleotides, LNA and/or PNA nucleotides or mixtures thereof.

### Antisense Composed Partially or Entirely of Nucleoside Analogues

In certain embodiments, the oligonucleic acid agent is an antisense oligonucleotide.

In certain embodiments, the oligonucleic acid agent comprises or is essentially composed of LNA moieties and comprises about 20 or fewer nucleotides.

In certain embodiments, the oligonucleic acid agent is essentially composed of LNA moieties and is described by the sequence ACC AAT CAG CTA AGC T (SEQ ID NO 008). In a particular embodiment, the nucleoside analogues of SEQ ID NO 008 are linked by phosphate esters. In a particular embodiment, the nucleoside analogues of SEQ ID NO 008 are linked by phosphothioate esters.

In certain embodiments, the oligonucleic acid agent for use in a method of treatment or prevention of heart disease comprises, or essentially consists of one or several peptide nucleic acid (PNA) moieties.

In certain embodiments the present invention includes a method of treating or preventing heart disease in a subject in need thereof comprising administering to the subject an oligonucleic acid agent directed against the miRNA miR27b-5p.

Inhibition of the miRNA miR27b-5p can be effected by sequence specific chemically modified oligonucleotides. In certain embodiments, the oligonucleic acid agent of the invention can comprise locked nucleic acid (LNA), in which the nucleic acid's ribose moiety is modified with an extra bridge connecting the 2′ oxygen and 4′ carbon, which locks the ribose in the 3′-endo conformation. Similarly, the oligonucleic acid agent of the invention may comprise PNA moieties. LNA and PNA have a higher binding energy to base-matched DNA or RNA, resulting in tighter binding. Such binding may add in antisense-based inhibition of the complementary miR27b-5p miRNA target.

In certain embodiments, the oligonucleic acid agent of the invention may include a phosphonate, a phosphorothioate or a phosphate ester phosphate backbone modification.

In certain embodiments, the oligonucleic acid agent of the invention may include ribonucleotides. Optionally, the oligonucleic acid agent of the invention may include deoxyribonucleotides.

In certain embodiments, the hybridizing sequence of the oligonucleic acid agent comprises ribonucleotides, deoxynucleotides, phosphothioate deoxynucleotides, phosphothioate ribonucleotides and/or 2′-O-methyl-modified phosphothioate ribonucleotides.

In certain embodiments, the oligonucleic acid agent comprises ribonucleotides and deoxyribonucleotides, in particular modified ribonucleotides and modified deoxyribonucleotides. A non-limiting example of a modification of deoxyribonucleotides and ribonucleotides are phosphorothioate modified linkages in the oligonucleotide backbone. A non-limiting example of a modification of ribonucleotides is a 2′-O to 4′-C bridge.

In certain embodiment the 2′-O/4′-C bridge is a five-membered, six-membered or seven membered bridged structure.

### Antisense Gapmers

In certain embodiments, the oligonucleic acid agent is a gapmer characterized by a central DNA block, the sequence of which is complementary to the miR27b-5p miRNA, and which is flanked on either side (5′ and 3′) by nuclease-resistant LNA sequences which are also complementary to the miR27b-5p miRNA. The central DNA block contains the RNase H activating domain, in other words is the part that lead the target DNA to be hydrolyzed. In certain embodiments, the flanking LNA is fully phosphorothioated.

In certain embodiments, the oligonucleic acid agent comprises 12-20 nucleotides. In certain particular embodiments, the oligonucleic acid agent comprises 14-16 nucleotides.

In certain embodiments, the hybridizing sequence of the oligonucleic acid agent according to the invention comprises 14,15 or 16 nucleotides.

In certain embodiments, the central deoxyribonucleotide oligomer block of the gapmer comprises at least 5 deoxyribonucleotides. In certain embodiments, the central deoxyribonucleotide oligomer block of the gapmer comprises 5 to 10 deoxyribonucleosides linked by phosphate ester bonds or thiophosphate ester bonds.

In certain embodiments, the central deoxyribonucleotide oligomer block of the gapmer comprises a phosphate backbone between the deoxyribonucleosides.

In certain embodiments, the oligonucleic acid agent comprises, or essentially consists of, a central block of 5 to 10 deoxyribonucleotides linked by phosphate ester bonds flanked on either side by 2′-O modified ribonucleotides or PNA oligomers. In certain embodiments, the oligonucleic acid agent comprises, or essentially consists of, a central block of 5 to 10 deoxyribonucleosides flanked by LNA nucleoside analogues. In certain particular embodiments, said LNA nucleoside analogues are linked by phosphothioate moieties.

In certain embodiments, the oligonucleic acid agent of the invention comprises or essentially consists of the sequence ACCA-atcagcta-AGCT (SEQ ID NO 005), wherein the capital letters signify nucleoside analogues, particularly LNA, more particularly LNA linked by phosphothioate esters, and the lower case letters signify DNA nucleosides linked by phosphate esters, and the link between a nucleoside analogue and a DNA nucleoside is selected from phosphate ester and thiophosphate.

### RNAi/siRNA/shRNA

In certain embodiments, the oligonucleic acid agent is a ribonucleic agent, particularly a siRNA or shRNA.

An RNA interference (RNAi) agent in the context of the present specification refers to a ribonucleotide oligomer that causes the degradation of its enhancer RNA (eRNA) target sequence.

In certain embodiments, the RNAi agents of the invention comprise, or consist of,
a single-stranded or double-stranded interfering ribonucleic acid oligomer or precursor thereof, comprising a sequence tract complementary to the targeted enhancer RNA molecule; or
a single-stranded or double-stranded antisense ribonucleic or deoxyribonucleic acid, comprising a sequence tract complementary to the targeted enhancer RNA molecule.

In certain embodiments, the sequence tract complementary to the targeted enhancer RNA molecule is a contiguous sequence tract 7, 8, 9, 10, 11, 12, 13, 14, 15, 16, 17, 18, 19, 20, 21 or 22 nucleotides in length.

In certain embodiments, the RNAi agents of the invention include, but are not limited to, small interfering RNAs (siRNAs), short hairpin RNAs (shRNAs), microRNAs and non-coding RNAs or the like, Morpholinos (phosphodiamidate morpholino oligomers) and Dicer substrate siRNAs (DsiRNAs, DsiRNAs are cleaved by the RNAse III class endoribonuclease Dicer into 21-23 base duplexes having 2-base 3′-overhangs), UsiRNAs (UsiRNAs are duplex siRNAs that are modified with non-nucleotide acyclic monomers, termed unlocked nucleobase analogues (UNA), where the bond between two adjacent carbon atoms of ribose is removed), self-delivering RNAs (sdRNAs) including rxRNA™ (RXi Pharmaceuticals, Westborough, Mass., USA).

In some embodiments, the RNAi agents of the invention comprise analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed partially of any of the above nucleotides, with the rest of the nucleotides being “native” ribonucleotides occurring in nature, or may be mixtures of different analogues, or may be entirely composed of one kind of analogue.

In certain embodiments, the oligonucleic agent is conjugated to, or encapsulated by, a nanoparticle, a virus and a lipid complex.

In certain embodiments, the oligonucleic acid agent is a gapmer comprising a central deoxyribonucleotide oligomer block flanked by nuclease resistant ribonucleotide analogues on either (5′ and 3′) side.

Within the scope of the present invention is a method for treating or preventing heart disease in a patient in need thereof, comprising administering to the patient an oligonucleic acid agent according to the invention.

Similarly, a dosage form for the prevention or treatment of heart disease is provided, comprising the oligonucleic acid agent according to the invention.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

In certain embodiments, the amount of the oligonucleic acid agent sufficient to reduce expression of miR27b-5p miRNA is of 1 nanomolar or less, 200 picomolar or less, 100 picomolar or less, 50 picomolar or less, 20 picomolar or less, 10 picomolar or less, 5 picomolar or less, 2, picomolar or less and 1 picomolar or less in the environment of the cell.

Sequences

SEQ ID NO 001 (miR27b-5p targeting sequence;
5′-3′, and nucleotide chemistry):
ACC AAT CAG CTA AGC T
SEQ ID NO 002 (miR27b-5p)
GCAGAACUUAGCCACUGUGAA
SEQ ID NO 003 (AAV9-fl/fl-shMthfd1I; sense):
TGAATGGTGTCAGAGAATTTTTCAAGAGAAAATTCTCTGACACCATTCTT
TTTTC
SEQ ID NO 004 (AAV9-fl/fl-shMthfd1I; antisense):
TCGAGAAAAAAGAATGGTGTCAGAGAATTTTCTCTTGAAAAATTCTCTGA
CACCATTCA
SEQ ID NO 005 ACCA-atcagcta-AGCT (upper case:
nucleoside analogue, particularly LNA linked by
thiophosphate; lower case: DNA; boundary:
phosphate or thiophosphate)
SEQ ID NO 006 (Primer sequence forward; mir27b):
GCATGCTGATTTGTGACTTGAG-3′
SEQ ID NO 007 (Primer sequence reverse; mir27b):
5′-CCTCTGTTCTCCAAACTGCAG-3′.
SEQ ID NO 008: (miR27b-5p targeting sequence;
5′-3′, LNA):
ACC AAT CAG CTA AGC T

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1A-B show the control of F₁F₀ ATP synthase on endoreplication and multinucleation in pathologic growth. Expression of genes coding for ATP synthase subunits and markers of cardiac hypertrophy and dysfunction (NPPA and NPPB) in left ventricular biopsies from hypertrophic cardiomyopathy (HCM) and aortic stenosis (AS) patients versus healthy controls and in ventricular biopsies from mice subjected to transaortic constriction (TAC) versus sham-operated animals. The echocardiographically obtained measurements of the inter-ventricular septum diameter (IVSD;d) and left-ventricular posterior wall diameter in diastole (LVPWD;d), as well as the fractional shortening (% FS) and ejection fraction (% EF) of TAC or sham operated animals is shown.

FIG. 1C-D shows biopsies of left ventricles from patients with HCM and aortic stenosis and healthy controls (c) or sham- and TAC-operated mice (d) assessed for denoted protein expression by immunoblotting.

FIG. 1E-F show the ADP/ATP ratio in ventricular biopsies from HCM and aortic stenosis patients versus healthy controls (e), and in ventricular biopsies from mice subjected to TAC versus sham-operated animals (f).

FIG. 1G shows left ventricular longitudinal sections from patients with aortic stenosis (AS) (n=3) and hypertrophic cardiomyopathy (HCM) (n=3) and healthy controls (n=3) stained for DAPI (blue), laminin (green) and α-actinin (red), and imaged by confocal microscopy.

FIG. 1H shows heart sections stained as in FIG. 1G and assessed for the percentage of mono-, bi-, or multinucleated cardiomyocytes.

FIG. 1I shows left ventricular longitudinal sections from sham- (n=3) and TAC (n=3)-operated mice stained for DAPI (blue), laminin (green) and α-actinin (red), and imaged by confocal microscopy.

FIG. 1J shows heart sections stained as in (i) were assessed for the ratio of mononucleated to multinucleated cardiomyocytes. (2 sections/heart were analyzed with 2 fields/section used for quantification. In total, 3 hearts were analyzed per group).

FIG. 1K shows schematic representation of the AAV9-fl/fl-shAtp5a1 virus before and after Cre-mediated recombination (left panel) and of the experimental timeline (right panel).

FIG. 1L shows an immunoblot of ventricular lysates from Mlc2v-cre⁺ and Mlc2v⁻ cre mice transduced with AAV9-fl/fl-shAtp5a1 using antibodies against denoted proteins.

FIG. 1M shows an ADP/ATP ratio measured in left ventricular samples of Mlc2v-cre⁺ and Mlc2v⁻ cre mice transduced with AAV9-fl/fl-shAtp5a1 11 weeks after AAV9 injection.

FIG. 1N shows left ventricular longitudinal sections from Mlc2v-cre⁺ (n=3) and Mlc2v-cre⁻ (n=3) mice transduced with AAV9-fl/fl-shAtp5a1 were stained for DAPI (blue), laminin (green) and a-actinin (red), and imaged by confocal microscopy.

FIG. 1O shows heart sections stained as in FIG. 1N assessed for the ratio of mono-, bi-, or multinucleated cardiomyocytes.

FIG. 1P shows representative images of left ventricles of Mlc2v-cre⁺ and Mlc2v⁻ cre mice transduced with AAV9-fl/fl-shAtp5a1 11 weeks after AAV9 injection.

FIG. 1Q shows representative images of H&E-stained histological sections of Mlc2v-cre⁺ and M/c2v⁻ cre mice transduced with AAV9-fl/fl-shAtp5a1 11 weeks after AAV9 injection.

FIG. 1R-S show left ventricular weight/body weight (LVW/BW) (R) and ejection fraction (S) of M/c2v-cre⁺ and Mlc2v⁻ cre mice transduced with AAV9-fl/fl-shAtp5a1 11 weeks after AAV9 injection.

FIG. 2A shows the expression of MIR27B in patients with hypertrophic cardiomyopathy (HCM) or aortic stenosis (AS)

FIG. 2B shows the schematic representation of the experimental timeline of Mlc2v⁻ cre mice transduced with AAV9-fl/fl-mir27b.

FIG. 2C shows representative images of left ventricles of Mlc2v-cre⁺ and Mlc2v⁻ cre mice injected with AAV9-fl/fl-mir27b viruses.

FIG. 2D shows representative images of H&E-stained histological sections of Mlc2v-cre⁺ and M/c2v-cre⁺ mice transduced with AAV9-fl/fl-mir27b viruses.

FIG. 2E-F show LVW/BW (e) and ejection fraction (f) of Mlc2v-cre⁺ and Mlc2v⁻ cre mice injected with AAV9-fl/fl-mir27b viruses.

FIG. 2G show the relative expression of mature miR27b-5p and miR27b-3p in Mlc2v-cre⁺ and M/c2v-cre⁺ mice injected with AAV9-fl/fl-mir27b viruses.

FIG. 2H shows an immunoblot of ventricular lysates from Mlc2v-cre⁺ and Mlc2v-cre⁻ mice transduced as in FIG. 2C-G using antibodies against denoted proteins.

FIG. 2I shows left ventricular longitudinal sections from Mlc2v-cre⁺ (n=3) and Mlc2v-cre⁻ (n=3) mice transduced with AAV9-fl/fl-mir27b, stained for DAPI (blue), laminin (green) and α-actinin (red) and imaged by confocal microscopy.

FIG. 2J shows heart sections stained as in FIG. 2I and assessed for the ratio of mononucleated to multinucleated cardiomyocytes.

FIG. 2K shows a schematic representation of the antagomir study in sham- and TAC-operated mice. Baseline echocardiography measurement was performed at day=−5, sham or TAC surgery at day=0, scrLNA and miR27b-5p LNAs delivered intraperitoneal (i.p) at day=49-52 post surgery and monitored by echocardiography at the indicated time points.

FIG. 2 L-M show representative images of left ventricles (FIG. 2L) and H&E-stained (FIG. 2M) histological sections.

FIG. 2N-P show longitudinal monitoring of LVW/BW (FIG. 2N), left ventricular internal diameter at end systole (LVID;s) (FIG. 2O) and ejection fraction (FIG. 2P) of C57BL/6J mice subjected to sham or TAC surgery and treated with either scrambled (scrLNA) or miR27b-5p LNAs.

FIG. 2Q-T show the relative expression of mature miR27b-5p (FIG. 2Q), miR27b-3p (FIG. 2R), Nppa and Nppb (FIG. 2S) and Atp5a1 mRNA (FIG. 2T) in C57BL/6J mice treated with scrLNA or miR27b-5p LNAs and subjected to either sham or TAC surgery by qPCR.

FIG. 2U shows an immunoblot of ventricular lysates from sham- and TAC operated C57BL/6J mice treated with either scrLNA or miR27b-5p LNAs using antibodies against denoted proteins.

FIG. 2V shows left ventricular longitudinal sections from sham- or TAC operated mice treated with either scrLNA or miR27b-5p LNAs stained for DAPI (blue), laminin (green) and α-actinin (red) and imaged by confocal microscopy.

FIG. 2W shows heart sections stained as in (v) were assessed for the ratio of mononucleated to multinucleated cardiomyocytes.

FIG. 2X shows Kaplan-Meier survival curves comparing mortality between mice subjected to sham or TAC surgery and treated with scrLNA or miR27b-5p LNAs. No mortality was observed in sham-operated animals.

FIG. 3A shows a schematic representation of de novo purine biosynthesis pathway showing the contribution of glycolysis and 1-carbon metabolism.

FIG. 3B shows the relative amount of formate in NRCs transduced and treated as denoted.

FIG. 3C shows relative mitochondrial ADP/ATP ratio in NRCs transduced and treated as denoted.

FIG. 3D shows a heat map of relative metabolite abundance in NRCs transduced and treated as indicated. Depicted are metabolites with log₂(fold change)>0.5 compared to control treatment and adjusted p value<0.01 in at least one treatment group compared to corresponding control (n=4 biological replicates per group).

FIG. 3E shows the relative amount of [¹⁴C]carbon derived from [¹⁴C]glucose, [¹⁴C]serine and [¹⁴C]glycine incorporated into nucleic acids in NRCs transduced and treated as denoted.

FIG. 4A-F show NRCs transduced and treated as indicated were stained with propidium iodide (PI) and assessed for polyploidy by flow cytometry coupled to imaging (a,c,e) and multinucleation quantified from images (b,d,f).

FIG. 4G-I show an evaluation of [³H]leucine incorporation in NRCs transduced and treated as indicated. Data is represented as incorporated radioactivity relative to control NRCs (set as 1.0).

FIG. 5A shows a schematic representation of the experimental timeline of Mlc2v⁻ cre mice transduced with AAV9-fl/fl-shMthfd1l and subjected to sham or TAC surgery.

FIG. 5B shows left ventricular longitudinal sections from sham- or TAC-operated Mlc2v-cre⁺ and Mlc2v⁻ cre mice injected with AAV9-fl/fl-shMthfd1l viruses were stained for DAPI (blue), laminin (green) and α-actinin (red), and imaged by confocal microscopy. (3 sections/heart were analyzed with 2-4 fields/section imaged with representative z-stack fields shown. In total, 3 hearts were analyzed per group). Arrows indicate nucleation of cardiomyocytes.

FIG. 5C shows heart sections stained as in (b) were assessed for the ratio of mononucleated to multinucleated cardiomyocytes.

FIG. 5D shows representative images of left ventricles of sham or TAC-operated Mlc2v-cre⁺ and Mlc2v⁻ cre mice injected with AAV9-fl/fl-shMthfd1l viruses.

FIG. 5E shows representative images of H&E-stained histological sections of sham or TAC-operated Mlc2v-cre⁺ and Mlc2v⁻ cre mice injected with AAV9-fl/fl-shMthfd1l viruses.

FIG. 5F-G shows LVW/BW (f) and ejection fraction (g) of sham or TAC-operated Mlc2v-cre⁺ and Mlc2v⁻ cre mice injected with AAV9-fl/fl-shMthfd1l viruses.

FIG. 5H shows an immunoblot of ventricular lysates from sham or TAC-operated Mlc2v-cre⁺ and Mlc2v⁻ cre mice transduced as in (b-g) using antibodies against denoted proteins.

FIG. 5I shows a schematic representation of the experimental timeline of Mlc2v⁻ cre mice co-transduced with AAV9-fl/fl-shAtp5a1 and AAV9-fl/fl-shMthfd1l.

FIG. 5J shows left ventricular longitudinal sections from Mlc2v-cre⁺ (n=3) and Mlc2v-cre⁻ (n=3) mice co-transduced with AAV9-fl/fl-shAtp5a1 and AAV9-fl/fl-shMthfd1l were stained for DAPI (blue), laminin (green) and α-actinin (red), and imaged by confocal microscopy.

FIG. 5K shows heart sections stained as in (j) were assessed for the ratio of mononucleated to multinucleated cardiomyocytes.

FIG. 5I shows representative images of left ventricles of Mlc2v-cre⁺ and Mlc2v⁻ cre mice co-transduced with AAV9-fl/fl-shAtp5a1 and AAV9-fl/fl-shMthfd1l.

FIG. 5M shows representative images of H&E-stained histological sections of Mlc2v-cre⁺ and M/c2v⁻ cre mice co-transduced with AAV9-fl/fl-shAtp5a1 and AAV9-fl/fl-shMthfd1l.

FIG. 5N-O shows LVW/BW (n) and ejection fraction (o) of Mlc2v-cre⁺ and Mlc2v⁻ cre mice co-transduced with AAV9-fl/fl-shAtp5a1 and AAV9-fl/fl-shMthfd1l.

FIG. 5P shows an immunoblot of ventricular lysates from Mlc2v-cre⁺ and Mlc2v-cre⁻ mice transduced as in j-p using antibodies against denoted proteins.

FIG. 6A shows an immunoblot of NRCs ectopically expressing a constitutively active form of AMPKα1 using antibodies against denoted proteins.

FIG. 6B shows an immunoprecipitation (IP) with either control IgG or anti-AMPKα antibody from NRCs ectopically expressing a constitutively active form of AMPKα1. The IPs were blotted using p-AMPKα and p-Rb antibodies.

FIG. 6C shows an immunoblot of NRCs ectopically expressing HIF1αΔODD and treated with either DMSO or the AMPK inhibitor compound C (CC) using antibodies against denoted proteins.

FIG. 6D shows an immunoprecipitation with either control IgG or anti-AMPKα antibody from NRCs ectopically expressing HIF1αΔODD and treated with either DMSO or the AMPK inhibitor CC. The IPs were blotted using p-AMPKα and p-Rb antibodies.

FIG. 6E shows an immunoblot of ventricular lysates from sham- or TAC operated mice treated with either scrLNA or miR27b-5p LNAs using antibodies against p-Rb and total Rb.

FIG. 6F shows an immunoblot of ventricular lysates from Mlc2v-cre⁺ and Mlc2v⁻ cre mice injected with AAV9-fl/fl-mir27b viruses using antibodies against p-Rb and total Rb.

FIG. 6G shows an immunoblot of ventricular lysates from Mlc2v-cre⁺ and Mlc2v-cre⁻ mice injected with AAV9-fl/fl-shAtp5a1 viruses using antibodies against p-Rb and total Rb.

FIG. 6H shows an immunoblot of ventricular lysates from sham- or TAC-operated Mlc2v-cre⁺ and Mlc2v⁻ cre mice injected with AAV9-fl/fl-shMthfd1l viruses using antibodies against p-Rb and total Rb.

FIG. 6I shows an immunoblot of ventricular lysates from Mlc2v-cre⁺ and Mlc2v-cre⁻ mice co-transduced with AAV9-fl/fl-shAtp5a1 and AAV9-fl/fl-shMthfd1l using antibodies against p-Rb and total Rb.

FIG. 6J shows an immunoblot of ventricular lysates from HCM and aortic stenosis patients and healthy controls using antibodies against p-Rb and total Rb.

FIG. 6K shows a heat map of relative expression of denoted genes in ventricular lysates from sham- or TAC operated mice treated with either scrLNA or miR27b-5p LNAs.

FIG. 6L shows a heat map of relative expression of denoted genes in ventricular lysates from M/c2v-cre⁺ and Mlc2v-cre⁻ mice injected with either AAV9-fl/fl-mir27b

FIG. 6M shows a heat map of relative expression of denoted genes in ventricular lysates from M/c2v-cre⁺ and Mlc2v⁻ cre mice subjected to sham or TAC surgery and injected with AAV9-fl/fl-shMthfd1l viruses.

FIG. 6N shows a heat map of relative expression of denoted genes in ventricular lysates from HCM and aortic stenosis patients and healthy controls.

FIG. 6O shows the transfection of E2F luciferase reporter in NRCs infected and treated as indicated. Data is normalized to control infected NRCs (set as 1.0).

FIG. 6P shows the transfection of E2F luciferase reporter in NRCs infected as indicated. Data is normalized to control infected NRCs (set as 1.0).

FIG. 6Q shows the proposed mechanism of F₁F₀ ATP synthase function in cardiac ploidy and growth control. Pathologic stress leads to the stabilization and accumulation of HIF1a and HIF1α-dependent induction of glycolytic genes and the microRNA MIR27B. MIR27B, in turn, binds to the 3′UTR of the alpha subunit of F₁F₀ ATP synthase (ATP5A1) thereby targeting it for degradation. As a consequence of F₁F₀ ATP synthase inhibition, the ADP/ATP ratio is elevated inside the mitochondrial matrix which, on one hand, activates the energy sensor AMPK. pAMPK in turn hyperphosphorylates Rb resulting in the release of the transcription factor E2F and induction of cyclins and CDKs expression. On the other hand, the accumulating mitochondrial ADP is rechanneled towards MTHFD1L, an enzyme involved in the mitochondrial one-carbon metabolism that uses ADP as a cofactor to catalyze the conversion from CHO-THF to format. Formate, that is particularly derived from serine serves as a 1-carbon donor for the de novo synthesis of purines. The increased de novo formation of purines as substrates for nucleic acid synthesis and the induction of cyclins and CDKs promote DNA replication, multinucleation and cardiac growth. CHO-THF denotes formyl-tetrahydrofolate, CH₂-THF denotes methylenetetrahydrofolate, 3-PG denotes 3-phosphoglycerate.

FIG. 7 a shows a Pearson's correlation coefficient of ATP synthase subunit genes presented in FIG. 1a (left panel) against expression of NPPB. (n=4 for healthy controls; n=10 for AS and n=11 for HCM).

FIG. 7 b shows quantification of ATP levels from ventricular lysates of sham- and TAC-operated animals. (n=5 for sham and n=6 for TAC; shown is mean±SEM; *** p<0.001; two-tailed unpaired t-test).

FIG. 7 c, Left ventricular sections from HCM (n=3) and aortic stenosis (n=3) patients and healthy controls (n=3) were stained with haematoxylin and eosin (H&E) and imaged by light microscopy. (2 sections/heart with 2 fields/section were surveyed with representative fields shown). Scale bar is 200 μm.

FIG. 7 d, Left ventricular sections from sham- (n=3) and TAC (n=3)-operated mice were stained with H&E and imaged by light microscopy. (3 sections/heart with 3-5 fields/section were surveyed with representative fields shown). Scale bar is 200 μm.

FIG. 7 e, Cardiomyocytes were isolated from sham- and TAC-operated mice and stained for DAPI and α-actinin. Cells were imaged by confocal microscopy and a representative z-stack image from 3 mice per group is shown. Scale bar is 20 μm.

FIG. 7 f, Adult cardiomyocytes stained as in (e) were assessed for the ratio of mononucleated to multinucleated cardiomyocytes. (At least 130 cells were quantified from n=3 mice per group).

FIG. 7 g, HW/BW in sham- and TAC-operated mice. (n=5 for sham and n=6 for TAC; * p<0.05; two-tailed unpaired t-test).

FIG. 7 h,i, Representative images of H&E (h) and picrosirius red (i) stained histological sections of sham- and TAC-operated mice. Scale bar is 500 μm.

FIG. 7 j, Relative expression of Col1a1, Col3a1 and TGFb1 mRNA in left ventricular lysates of sham and TAC-operated mice. Data is normalized to sham-operated animals (set as 1.0). (n=5 for sham and n=6 for TAC; results shown are the mean±SEM; * p<0.05; two-tailed unpaired t-test)

FIG. 8 a, Relative expression of Atp5a1 mRNA in Mlc2v-cre⁺ and Mlc2v⁻ cre mice transduced with AAV9-fl/fl-shAtp5a1 11 weeks after AAV9 injection. Data is normalized to Mlc2v⁻ cre mice transduced with AAV9-fl/fl-shAtp5a1 (set as 1.0). (n=8 for Mlc2v-cre⁻ and n=9 for Mlc2v-cre⁺; shown is mean±SEM; *** p<0.001; two-tailed unpaired t-test).

FIG. 8b, Quantification of ATP levels from ventricular lysates of Mlc2v-cre⁺ and Mlc2v⁻ cre mice transduced with AAV9-fl/fl-shAtp5a1 (n=8 for Mlc2v-cre⁻ and n=9 for Mlc2v-cre⁺; shown is mean±SEM; * p<0.05; two-tailed unpaired t-test).

FIG. 8c, Left ventricular sections from Mlc2v-cre⁺ (n=3) and Mlc2v-cre⁻ (n=3) mice injected with AAV9-fl/fl-shAtp5a1 viruses were stained with H&E and imaged by light microscopy. (3 sections/heart with 3-5 fields/section were surveyed with representative fields shown). Scale bar is 200 μm.

FIG. 8d, Cardiomyocytes were isolated from Mlc2v-cre⁺ and Mlc2v⁻ cre mice injected with AAV9-fl/fl-shAtp5a1 viruses and stained for DAPI and α-actinin. Cells were imaged by confocal microscopy and a representative z-stack image from 3 mice per group is shown. Scale bar is 20 μm.

FIG. 8e, Adult cardiomyocytes stained as in (d) were assessed for the ratio of mononucleated to multinucleated cardiomyocytes. (At least 150 cells were quantified from n=3 mice per group).

FIG. 8f, Relative expression of Nppa and Nppb mRNA in Mlc2v-cre⁺ and Mlc2v⁻ cre mice transduced with AAV9-fl/fl-shAtp5a1 11 weeks after AAV9 injection. Data is normalized to M/c2v⁻ cre mice transduced with AAV9-fl/fl-shAtp5a1 (set as 1.0). (n=8 for Mlc2v-cre⁻ and n=9 for Mlc2v-cre⁺; shown is mean±SEM; * p<0.05; two-tailed unpaired t-test).

FIG. 8g, LVID;s in Mlc2v-cre⁺ and Mlc2v⁻ cre mice transduced with AAV9-fl/fl-shAtp5a1 11 weeks after AAV9 injection. (n=8 for Mlc2v-cre⁻ and n=9 for Mlc2v-cre⁺; shown is mean±SEM; ** p<0.01; two-tailed unpaired t-test).

FIG. 8 h, Representative images of picrosirius red-stained histological sections of Mlc2v-cre*and Mlc2v-cre⁻ mice transduced with AAV9-fl/fl-shAtp5a1 viruses. Scale bar is 500 μm.

FIG. 8i, Relative expression of Col1a1, Col3a1 and TGFb1 mRNA in left ventricular lysates of M/c2v-cre⁺ and Mlc2v⁻ cre mice transduced with AAV9-fl/fl-shAtp5a1 viruses. Data is normalized to Mlc2v⁻ cre mice (set as 1.0). (n=8 for Mlc2v-cre⁻ and n=9 for Mlc2v-cre⁺ mice; results shown are the mean±SEM; * p<0.05; two-tailed unpaired t-test).

FIG. 8j, Relative expression of ATP5A1 mRNA in iPSC-derived human cardiomyocytes (iPSC-hCM) transduced with lentivirus expressing non-silencing shRNA (nsRNA) or shRNA against ATP5A1 (shATP5A1). Data is normalized to control NRCs expressing nsRNA (set as 1.0). (n=3 biological replicates per group; results shown are the mean±SD; *** p<0.001; two-tailed unpaired t-test).

FIG. 8k, ADP/ATP ratio in iPSC-hCM transduced with lentivirus expressing nsRNA or shATP5A1. (n=3 biological replicates per group; shown is mean±SD; ** p<0.01; two-tailed unpaired t-test).

FIG. 8l,m, Quantification of ADP (1) and ATP (m) levels from iPSC-hCM transduced with lentivirus expressing nsRNA or shATP5A1. (n=3 for both groups; shown is mean±SD; ** p<0.01; two-tailed unpaired t-test).

FIG. 8n, iPSC-hCM transduced with lentivirus expressing nsRNA or shATP5A1 were stained for DAPI and phalloidin, and imaged by confocal microscopy. Representative fields from 3 biological replicates are shown. Scale bar is 20 μm.

FIG. 8o, Quantification of percentage of multinucleated cells in iPSC-hCM treated as in (n) (n=3 biological replicates with approximately 100 cells analyzed per experiment and condition; shown is mean±SD; * p<0.05; two-tailed unpaired t-test).

FIG. 8p, iPSC-hCM treated as in (n) were assessed for cell size using ImageJ. (n=3 independent experiments with approximately 100 cells analyzed per experiment and condition; shown is mean±SD; * p<0.05; two-tailed unpaired t-test).

FIG. 8q, Relative expression of Atp5a1 mRNA in NRCs transduced with lentivirus expressing nsRNA or shAtp5a1. Data is normalized to control NRCs expressing nsRNA (set as 1.0). (n=3 biological replicates per group; results shown are the mean±SD; *** p<0.001; two-tailed unpaired t-test).

FIG. 8r, NRCs transduced with nsRNA or shAtp5a1 were assessed for Atp5a1 protein levels by immunoblotting. Loading is normalized to cardiac actin.

FIG. 8s, ADP/ATP ratio in NRCs transduced with lentivirus expressing nsRNA or Atp5a1 shRNA as indicated. (n=3 biological replicates per group; shown is mean±SD; ** p<0.01; two-tailed unpaired t-test).

FIG. 8t,u, Quantification of ADP (t) and ATP (u) levels from NRCs transduced with lentivirus expressing nsRNA or Atp5a1 shRNA. (n=3 for both groups; shown is mean±SD; ** p<0.01; two-tailed unpaired t-test).

FIG. 8v, NRCs transduced with lentivirus expressing nsRNA or Atp5a1 shRNA were stained for phalloidin and DAPI, and imaged by confocal microscopy. Representative fields from n=3 biological replicates are shown. Scale bar is 20 μm.

FIG. 8w, Quantification of the percentage of multinucleated cells in NRCs treated as in (v) (n=3 biological replicates with approximately 100 cells analyzed per experiment and condition; shown is mean±SD; * p<0.05; two-tailed unpaired t-test).

FIG. 8x, NRCs treated as in (v) were assessed for cell size using ImageJ. (n=3 independent experiments with approximately 100 cells analyzed per experiment and condition; shown is mean±SD; * p<0.05; two-tailed unpaired t-test).

FIG. 8y, Evaluation of [³H]leucine incorporation in NRCs transduced with lentivirus expressing nsRNA or Atp5a1 shRNA. Data is represented as incorporated radioactivity relative to control NRCs expressing nsRNA (set as 1.0). (n=4 biological replicates per group; results shown are the mean±SEM; * p<0.05; two-tailed unpaired t-test).

FIG. 9 a,b, Relative expression of Atp5a1 mRNA in ventricles of control (Hif1α f/fl) and Hif1α cKO mice subjected to sham or TAC surgery (a), or control (Vhl fl/fl) and Vhl cKO mice (b). Data is normalized to controls (set as 1.0). (n=5 mice per group for (a) and (b); shown is mean±SEM; *, % p<0.05; ** p<0.01; one-way ANOVA and Bonferroni correction for (a) and two-tailed unpaired t-test for (b)).

c, Ventricular lysates of sham- or TAC-operated control (Hif1α f/fl) and Hif1α cKO mice (left panel), or control (Vhl fl/fl) and Vhl cKO mice (right panel) were processed for immunoblotting with antibodies against Hif1α and Atp5a1. Loading is normalized to cardiac actin.

FIG. 9 d,e, Quantification of ATP amount (d) and ejection fraction (e) in left ventricular biopsies from control (Hif1α fl/fl) and ventricle-specific Hif1α conditional knockout (Hif1α cKO) mice subjected to sham or TAC surgery (d). (n=5 mice per group for (d) and (e); shown is mean±SEM; *, % p<0.05; one-way ANOVA and Bonferroni correction).

FIG. 9 f,g, Quantification of ATP amount in left ventricular biopsies (f) and ejection fraction (g) of ventricle-specific Vhl conditional knockout (Vhl cKO) and respective control (Vhl fl/fl) mice (g). (n=5 mice per group for (f) and (g); shown is mean±SEM; * p<0.05; ** p<0.01; two-tailed unpaired t-test).

FIG. 9 h, Expression of miRNAs containing putative HRE(s) in their promoter (p<0.0001 between technical quadruplicates, and p<0.05 of the biological triplicates with average signal intensity of 1-fold over background) from ventricular lysates of Hif1α cKO mice subjected to TAC surgery versus TAC-operated control animals, or Vhl cKO mice versus control animals.

FIG. 9 i, Relative expression of mir27b, Vegfa and Ldha mRNA in ventricles of control (Hif1α fl/fl) and Hif1α cKO mice subjected to sham or TAC surgery. Data is normalized to sham-operated controls (set as 1.0). (n=5 mice per group; shown is mean±SEM; *, % p<0.05; one-way ANOVA and Bonferroni correction).

FIG. 9 j, Relative expression of mir27b RNA in ventricles of control (Vhl fl/fl) and Vhl cKO mice. Data is normalized to control mice (set as 1.0). (n=5 mice per group; shown is mean±SEM; *** p<0.001; two-tailed unpaired t-test).

FIG. 9 k-m, Relative expression levels of AmpO (k), mature miR27b-3p, miR23b-3p and miR24-3p (I), and miR27b-5p, miR23b-5p and miR24-5p (m) from ventricular lysates of wildtype C57BL/6J mice subjected to sham or TAC surgery. Data is normalized to sham-operated control mice (set as 1.0). (n=5 for sham and n=8 for TAC; data shown is mean±SEM; * p<0.05; ** p<0.01; *** p<0.001; two-tailed unpaired t-test).

FIG. 9 n-p, Relative expression of AmpO (n), mature miR27b-3p, miR23b-3p and miR24-3p (o), and miR27b-5p, miR23b-5p and miR24-5p (p) in NRCs ectopically expressing an empty control vector or HIF1αΔODD. Data is normalized to NRCs transduced with an empty control vector (set as 1.0). (n=5 biological replicates per group; shown is mean±SEM; ** p<0.01; two-tailed unpaired t-test).

FIG. 9 q, Evaluation of [³H]leucine incorporation in NRCs treated with T3 or PBS (mock). Data is represented as incorporated radioactivity relative to mock-treated NRCs (set as 1.0). (n=4 biological replicates per group; results shown are the mean±SEM; *** p<0.001; two-tailed unpaired t-test).

FIG. 9 r-t, Relative expression of AmpO (r), mature miR27b-3p, miR23b-3p and miR24-3p (s), and miR27b-5p, miR23b-5p and miR24-5p (t) in NRCs treated with PBS (mock) or T3. Data is normalized to mock-treated NRCs (set as 1.0). (n=5 biological replicates per group; shown is mean±SEM; *** p<0.001; two-tailed unpaired t-test).

FIG. 9 u, Relative expression of Hif1a, mir27b, Glut1 and Vegfa mRNA in NRCs stimulated with T3 and transduced with non-silencing control shRNA (nsRNA) or shRNA against Hif1α (shHif1α). Data is normalized to control NRCs infected with nsRNA (set as 1.0). (n=3 biological replicates per group; shown is mean±SD; *, ç, % p<0.05; one-way ANOVA and Bonferroni correction).

FIG. 9 v, Sequence of the human, monkey, possum, rat and mouse mir27b promoter harboring a conserved HRE located 162 bp upstream of the precursor mir27b transcription start site. HRE is shown in red, with the core HRE motif capitalized.

FIG. 9 w, Co-transfection of wildtype (wt) or HRE-mutated (mut) mir27b promoters fused to luciferase with different doses of either an empty control vector or HIF1αΔODD. Data is normalized to wildtype promoter transfected with empty control vector (set as 1.0). (n=4 biological replicates per group; data shown is mean±SEM; * p<0.05; one-way ANOVA followed by Dunnett's post test).

FIG. 9 x, Co-transfection of mir23b and mir24-1 promoters fused to luciferase with different doses of either an empty vector control or HIF1α A ODD. Data is normalized to luciferase vector transfected with empty control vector (set as 1.0). (n=4 biological replicates per group; data shown is mean±SEM; * p<0.05; one-way ANOVA followed by Dunnett's post test).

FIG. 9 y, NRCs were transduced with lentivirus expressing either a nsRNA or shRNA against Vhl (shVhl) and processed for chromatin immunoprecipitation with a HIF1α-specific antibody (IP: HIF1α) or with a control isotype-matched antibody (IgG control). Hif1α promoter binding was analyzed by qPCR. Data shown is relative to chromatin immunoprecipitation with Ig control antibody on nuclear lysates from NRCs expressing nsRNA (set as 1.0). (n=3 biological replicates per group; data shown is mean±SD; *** p<0.001; two-tailed unpaired t-test).

FIG. 9 z, NRCs transduced with nsRNA or shVhl were assessed for Hif1α and Vhl protein levels by immunoblotting. Loading is normalized to cardiac actin.

FIG. 10 a, Target gene recognition motif in the 3′UTR of human and mouse Atp5a1. The miR27b-5p seed sequence and corresponding target region on Atp5a1 are indicated by alignment.

FIG. 10 b, Co-transfection of wildtype (wt) or mir27b binding site-mutated (mut) Atp5a1 3′UTR fused to luciferase with different doses of control, miR27b-3p or miR27b-5p mimics. Data is normalized to wildtype promoter transfected with control mimics (set as 1.0). (n=4 biological replicates per group; data shown is mean±SEM; ** p<0.01; *** p<0.001; one-way ANOVA followed by Dunnett's post test).

FIG. 10 c, NRCs transduced with empty control vector or ectopic mir27b were assessed for Atp5a1 protein levels by immunoblotting. Loading is normalized to cardiac actin.

FIG. 10 d,e, Relative expression of mir27b precursor (d) and mature miR27b-3p and miR27b-5p (e) in NRCs ectopically expressing an empty control vector or mir27b. Data is normalized to NRCs transduced with an empty control vector (set as 1.0). (n=3 biological replicates per group; shown is mean±SD; * p<0.05; *** p<0.001; two-tailed unpaired t-test).

FIG. 10 f, Relative expression level of Atp5a1 mRNA from NRCs transfected with different concentrations of control, miR27b-3p and miR27b-5p mimics. Data is normalized to control mimics (set as 1.0). (n=3 replicates per group; data shown is mean±SD; * p<0.05; *** p<0.001; one-way ANOVA followed by Dunnett's post test).

FIG. 10 g,h, Relative expression of mature miR27b-5p (g) and miR27b-3p (h) in NRCs transduced with an empty vector control or HIF1αΔODD and treated with either scrLNA or miR27b-5p LNAs. Data is normalized to NRCs transduced with an empty control vector (set as 1.0). (n=5 biological replicates per group; shown is mean±SEM; *, ç, % p<0.05; one-way ANOVA and Bonferroni correction).

FIG. 10 i, Immunoblot for Hif1α and Atp5a1 expression in NRCs transduced with lentivirus expressing empty control vector or HIF1αΔODD and treated with either scrambled control (scrLNA) or miR27b-5p LNAs. Loading is normalized to cardiac actin.

FIG. 10 j,k, Relative expression of mature miR27b-5p (j) and miR27b-3p (k) in NRCs treated with PBS (mock) or T3 in the presence of a scrLNA or miR27b-5p LNA. Data is normalized to mock-treated cells (set as 1.0). (n=5 biological replicates per group; shown is mean±SEM; *, g, % p<0.05; one-way ANOVA and Bonferroni correction).

FIG. 10 l, NRCs treated with PBS (mock) or T3 in the presence of scrLNA or miR27b-5p LNA were assessed for Hif1a, mir27b and Atp5a1 RNA levels. Data is normalized to NRCs treated with scrLNA (set as 1.0). (n=4 biological replicates per group; shown is mean±SEM; *, ç, % p<0.05; one-way ANOVA and Bonferroni correction).

FIG. 10 m, NRCs treated as in (1) were assessed for Atp5a1 protein levels by immunoblotting. Loading is normalized to cardiac actin.

FIG. 10 n,o, ATP synthase enzymatic activity in NRCs transduced and treated as indicated. Data is normalized to control sets (set as 1.0). (n=5 biological replicates per group; results shown are the mean±SEM; *, % p<0.05; ** p<0.01; *** p<0.001; two-tailed unpaired t-test (n) or one-way ANOVA followed by Bonferroni correction (o).

FIG. 10 p, NRCs transduced with empty control vector or ectopic ATP5A1 were assessed for Atp5a1 protein levels by immunoblotting. Loading is normalized to cardiac actin.

FIG. 11 a, Relative expression of Nppa and Nppb mRNA in Mlc2v-cre⁺ and Mlc2v⁻ cre mice transduced with AAV9-fl/fl-mir27b 11 weeks after AAV9 injection. Data is normalized to Mlc2v-cre mice transduced with AAV9-fl/fl-mir27b (set as 1.0). (n=6 for Mlc2v-cre⁻ and n=8 for Mlc2v-cre⁺; shown is mean±SEM; * p<0.05; two-tailed unpaired t-test).

FIG. 11 b, LVID;s in Mlc2v-cre⁺ and Mlc2v-cre⁻ mice transduced with AAV9-fl/fl-mir27b 11 weeks after AAV9 injection. (n=6 for Mlc2v-cre⁻ and n=8 for Mlc2v-cre⁺; shown is mean±SEM; * p<0.05; two-tailed unpaired t-test).

FIG. 11 c, Relative expression of Atp5a1 mRNA in Mlc2v-cre⁺ and Mlc2v⁻ cre mice transduced with AAV9-fl/fl-mir27b 11 weeks after AAV9 injection by qPCR. Data is normalized to Mlc2v-cre mice transduced with AAV9-fl/fl-mir27b (set as 1.0). (n=6 for Mlc2v-cre⁻ and n=8 for Mlc2v-cre⁺ mice; shown is mean±SEM; *** p<0.001; two-tailed unpaired t-test).

FIG. 11 d, Left ventricular sections from Mlc2v-cre⁺ and Mlc2v⁻ cre mice transduced with AAV9-fl/fl-mir27b were stained with H&E and imaged by light microscopy 11 weeks after AAV9 injection. (3 sections/heart with 3-5 fields/section were surveyed with representative fields shown. In total, 3 hearts were surveyed per group). Scale bar is 200 μm.

FIG. 11 e, Cardiomyocytes were isolated from Mlc2v-cre⁺ and Mlc2v⁻ cre mice injected with AAV9-fl/fl-mir27b viruses and stained for DAPI and α-actinin. Cells were imaged by confocal microscopy and a representative z-stack image from 3 mice per group is shown. Scale bar is 20 μm.

FIG. 11 f, Adult cardiomyocytes stained as in (e) were assessed for the ratio of mononucleated to multinucleated cardiomyocytes. (At least 160 cells were analyzed from n=3 mice per group).

FIG. 11 g, Representative images of picrosirius red-stained histological sections of Mlc2v-cre⁺ and Mlc2v⁻ cre mice transduced with AAV9-fl/fl-mir27b viruses. Scale bar is 500 μm.

FIG. 11 h, Relative expression of Col1a1, Col3a1 and TGFb1 mRNA in left ventricular lysates of Mlc2v-cre⁺ and Mlc2v⁻ cre mice injected with AAV9-fl/fl-mir27b viruses. Data is normalized to Mlc2v⁻ cre mice (set as 1.0). (n=6 for Mlc2v-cre⁻ and n=8 for Mlc2v-cre⁺ mice; results shown are the mean±SEM; * p<0.05; two-tailed unpaired t-test).

FIG. 11 i, Longitudinal monitoring of aortic velocity of C57BL/6J mice subjected to sham or TAC surgery and treated with either scrambled (scrLNA) or miR27b-5p LNAs. Arrows in panels indicate LNA injections. (n=5 for sham scrLNA, n=5 for sham miR27b-5p LNA, n=7 for TAC scrLNA, n=8 for TAC miR27b-5p LNA; shown is mean±SEM).

FIG. 11 j, Left ventricular sections from C57BL/6J mice subjected to sham or TAC surgery and treated with either scrLNA or miR27b-5p LNAs were stained with H&E and imaged by light microscopy. (3 sections/heart with 2-4 fields/section were surveyed with representative fields shown. In total, 3 hearts were surveyed per group). Scale bar is 200 μm.

FIG. 11 k, Cardiomyocytes were isolated from C57BL/6J mice subjected to sham or TAC surgery and treated with either scrLNA or miR27b-5p LNAs and stained for DAPI and α-actinin. Cells were imaged by confocal microscopy and a representative z-stack image from 3 mice per group is shown. Scale bar is 20 μm.

FIG. 11 l, Adult cardiomyocytes stained as in (k) were assessed for the ratio of mononucleated to multinucleated cardiomyocytes. (At least 150 cells were analyzed from n=3 mice per group).

FIG. 11 m, Representative images of picrosirius red-stained histological sections of C57BL/6J mice subjected to sham or TAC surgery and treated with either scrLNA or miR27b-5p LNAs. Scale bar is 500 μm.

FIG. 11 n, Relative expression of Col1a1, Col3a1 and TGFb1 mRNA in left ventricular lysates of C57BL/6J mice treated with scrLNA or miR27b-5p LNAs and subjected to either sham or TAC surgery by qPCR. Data is normalized to sham-operated mice treated with scrLNA (set as 1.0). (n=5 for sham scrLNA, n=5 for sham miR27b-5p LNA, n=7 for TAC scrLNA, n=8 for TAC miR27b-5p LNA; shown is mean±SEM; *, % p<0.05; one-way ANOVA and Bonferroni correction).

FIG. 12 a, Relative expression of Mthfd1l mRNA in NRCs expressing empty control vector or ectopic MTHFD1L. Data is normalized to NRCs expressing empty control vector (set as 1.0). (n=3 biological replicates per group; results shown are the mean±SD; *** p<0.05; two-tailed unpaired t-test).

FIG. 12 b, Immunoblot of NRCs transduced with lentivirus expressing empty control vector or MTHFD1L using antibodies against Mthfd1l. Loading is normalized to cardiac actin.

FIG. 12 c-h, Quantification of ATP (c,e,g) and ADP (d,e,h) levels in NRCs transduced and treated as indicated. (n=4 biological replicates per group; results shown are the mean±SD; *, % p<0.05; one-way ANOVA followed by Bonferroni correction).

FIG. 13 a, Evaluation of [³H]leucine incorporation in NRCs stimulated with T3 and treated with scrLNA or miR27b-5p LNAs. Data is represented as incorporated radioactivity relative to control (mock) NRCs treated with scrLNAs (set as 1.0). (n=4 biological replicates per group; results shown are the mean±SEM; *, % p<0.05; one-way ANOVA followed by Bonferroni correction).

FIG. 13 b, Relative amount of formate in NRCs stimulated with T3 and treated with scrLNA or miR27b-5p LNAs. (n=5 biological replicates per group; results shown are the mean±SEM; *,% p<0.05; one-way ANOVA followed by Bonferroni correction).

FIG. 13 c, Relative ADP/ATP ratio in NRCs stimulated with T3 and treated with scrLNA or miR27b-5p LNAs. (n=5 biological replicates per group; results shown are the mean±SEM; *,% p<0.05; one-way ANOVA followed by Bonferroni correction).

FIG. 13 d, Heat map of relative metabolite abundance in NRCs stimulated with T3 and treated with scrLNA or miR27b-5p LNAs. Depicted are metabolites with log₂(fold change)>0.4 and adjusted p value<0.01 in at least one treatment group compared to corresponding control (n=4 biological replicates per group).

FIG. 13 e, Relative amount of [¹⁴C]carbon derived from [¹⁴C]glucose, [¹⁴C]serine and [¹⁴C]glycine incorporated into nucleic acids in NRCs stimulated with T3 and treated with scrLNA or miR27b-5p LNAs. (n=5 biological replicates per group; results shown are the mean±SEM; * p<0.05; one-way ANOVA followed by Bonferroni correction).

FIG. 14 a, Differential analysis of metabolites in left ventricles of shm or TAC-operated C57BL/6J mice injected with scrLNA or mir27b-5p L C57BL/6J miceNA; circle seize reflects—log₁₀(adj.p-value); circle color reflects log 2 fold change compared to denoted control (n=6 biological replicates per group).

FIG. 14 b, Hierarchical cluster analysis of metabolites in denoted samples measured in duplicates (n=6 biological replicates per group).

FIG. 15 a, Principal Component Analysis (PCA) of denoted cohorts.

FIG. 15 b,c. Principal Component Analysis performed separately on left ventricular samples of sham or TAC treated mice with active (scrambled, b) or LNA-mediated repressed miR27b-5p (c). Arrows represent lipid species that concur to explain 15% of the variance encompassed by PC1 (Wilcoxon non-parametric signed-rank test; n=5 per group).

FIG. 15 d, Barplot of the saturation profiles in the denoted four cohorts (mean+/−SD). Lipids were grouped according to the number of double bonds (db), and all species exceeding 6 db were gathered in a single group (6+). Analyzed lipids include the following 19 lipid classes cholesteryl esters (CE), ceramides (Cer), cholesterols (Chol), cardiolipins (CL), diacylglycerols (DAG), lyso-phosphatides (LPA), lyso-phosphatidylcholines (LPC), Lyso-phosphatidylehanolamines (LPE), ether linked LPE (LPE-O⁻), lyso-phosphatidylinositols (LPI), phosphatidylcholines (PC), ether linked PC (PC-O⁻), phosphatidylethanolamines (PE), ether linked PE (PE-O⁻), phosphatidylglycerols (PG), phosphatidylinositols (PI), phosphatidylserines (PS), sphingomyelins (SM) and triacylglycerols. (adj. p-value<0.001***, adj. p-value<0.01**, adj. p-value<0.1*; Wilcoxon non-parametric signed-rank test; n=5 biological samples per group)

FIG. 15 e, Barplot of the total length profiles in the four denoted cohorts (mean+/−SD). Lipids (as analysed in d) were grouped according to the total length of their acyl chains, i.e. number of carbon atoms. Significant comparisons are indicated with asterisks (adj. p-value<0.001***, adj. p-value<0.01**, adj. p-value<0.1*; Wilcoxon non-parametric signed-rank test; n=5 biological samples per group).

FIG. 15 f, Mean mol % abundance of lipid species in left ventricular biopsies of scrLNA treated TAC-operated mice minus mean mol % abundance of lipid species in sham-controls with.

FIG. 15 g, Mean mol % abundance of lipid species in TAC-operated mice injected with miR27b-5p LNA minus mean mol % abundance of lipid species in sham-controls with repressed (LNA) miR27b-5p.

FIG. 15 h, Relative expression of indicated mRNAs in C57BL/6J mice treated with scrLNA or miR27b-5p LNAs and subjected to either sham or TAC surgery by qPCR. Data is normalized to sham-operated mice treated with scrLNA (set as 1.0). (n=5 for sham scrLNA, n=5 for sham miR27b-5p LNA, n=7 for TAC scrLNA, n=8 for TAC miR27b-5p LNA; shown is mean±SEM; *, % p<0.05; one-way ANOVA and Bonferroni correction).

FIG. 15 i, Oxidation of [1-¹⁴C]-palmitate to ¹⁴CO₂ was measured in left ventricular biopsies from C57BL/6J mice subjected to sham or TAC surgery and treated with either scrLNA or miR27b-5p LNAs. All values were normalised to the protein content. (n=5 for sham scrLNA, n=5 for sham miR27b-5p LNA, n=6 for TAC scrLNA, n=6 for TAC miR27b-5p LNA; shown is mean±SD; *, % p<0.05; one-way ANOVA and Bonferroni correction).

FIG. 16 a,b, Quantification of ADP (a) and ATP (b) levels in NRCs treated with PBS or 10 μM ADP for 3 days. (n=4 biological replicates per group; results shown are the mean±SD; *, % p<0.05; one-way ANOVA followed by Bonferroni correction).

FIG. 16 c,d, Quantification of ADP (c) and ATP (d) levels in NRCs treated with PBS or 20 nM oligomycin. (n=3 biological replicates per group; results shown are the mean±SD; *, % p<0.05; one-way ANOVA followed by Bonferroni correction).

FIG. 16 e, NRCs treated with PBS or 10 μM ADP for 3 days were stained for DAPI and α-actinin and imaged by confocal microscopy. Representative fields of three independent experiments are shown. Scale bar is 5 μm.

FIG. 16 f, NRCs treated with PBS oroligomycin were stained for DAPI and α-actinin and imaged by confocal microscopy. An average of 4 fields per condition and experiment were imaged and representative fields of three independent experiments are shown. Scale bar is 50 μm.

FIG. 16 g,h, NRCs treated and imaged as in (e,f) were assessed for multinucleation. (At least 110 cells were quantified per sample from n=3 independent experiments; shown is mean±SD; ** p<0.01; two-tailed unpaired t-test).

FIG. 16 i,j, NRCs treated and imaged as in (e,f) were assessed for cell surface area using ImageJ. (At least 110 cells were quantified per sample from n=3 independent experiments; shown is mean±SD; * p<0.05; ** p<0.01; two-tailed unpaired t-test).

FIG. 17 a-d, NRCs transduced with the indicated lentiviruses were stained for the cardiac-specific marker α-actinin, DAPI and phospho-Histone H3 (p-Histone H3) for assessment of cell mitosis and imaged by confocal microscopy. p-Histone H3 positive cardiomyocytes were quantified. 3 biological replicates with 3-4 fields/replicate were analyzed per condition with representative fields shown. Scale bar is 100 μm.

FIG. 17 e, Relative expression of Mthfd1l mRNA in NRCs expressing nsRNA or shMthfd1l. Data is normalized to NRCs expressing nsRNA (set as 1.0). (n=3 biological replicates per group; results shown are the mean±SD; *** p<0.05; two-tailed unpaired t-test).

FIG. 17 f, Immunoblot of NRCs transduced with lentivirus expressing nsRNA or shMthfd1l using an antibody against Mthfd1l. Loading is normalized to cardiac actin.

FIG. 17 g,h, NRCs transduced with the indicated lentiviruses were stained for the cardiac-specific marker α-actinin, DAPI and phospho-Histone H3 (p-Histone H3) for assessment of cell mitosis and imaged by confocal microscopy. p-Histone H3 positive cardiomyocytes were quantified. 3 biological replicates with 3-4 fields/replicate were analyzed per condition with representative fields shown. Scale bar is 100 μm.

FIG. 18 a, Left ventricular sections from sham- or TAC-operated Mlc2v-cre⁺ and Mlc2v-cre mice transduced with AAV9-fl/fl-shMthfd1l were stained with H&E and imaged by light microscopy 11 weeks post AAV9 injection. (3 sections/heart with 2-4 fields/section were surveyed with representative fields shown. In total, 3 hearts were surveyed per group). Scale bar is 200 μm.

FIG. 18 b, Left ventricular internal dimension at systole (LVID;s) in sham- or TAC-operated M/c2v-cre⁺ and Mlc2v⁻ cre mice transduced with AAV9-fl/fl-shMthfd1l 11 weeks post AAV9 injection. (n=4 for sham Mlc2v-cre-, n=5 for sham Mlc2v-cre⁺, n=8 for TAC Mlc2v-cre⁻ and n=7 for TAC Mlc2v-cre⁺ mice; shown is mean±SEM; *, % p<0.05; one-way ANOVA followed by Bonferroni correction).

FIG. 18 c,d, Relative expression of Nppa and Nppb (c) and Mthfd1l mRNA (d) from ventricular lysates obtained from sham- or TAC-operated Mlc2v-cre⁺ and Mlc2v-cre⁻ mice transduced with AAV9-fl/fl-shMthfd1l 11 weeks post AAV9 injection. Data is normalized to sham-operated M/c2v⁻ cre mice (set as 1.0). (n=4 for sham Mlc2v-cre, n=5 for sham Mlc2v-cre⁺, n=8 for TAC M/c2v-cre and n=7 for TAC Mlc2v-cre*mice; shown is mean±SEM; *, % p<0.05; one-way ANOVA followed by Bonferroni correction).

FIG. 18 e, Cardiomyocytes were isolated from sham- or TAC-operated Mlc2v-cre⁺ and Mlc2v-cre⁻ mice transduced with AAV9-fl/fl-shMthfd1l and stained for DAPI and α-actinin. Cells were imaged by confocal microscopy and a representative z-stack image from 3 mice per group is shown. Scale bar is 20 μm.

FIG. 18 f, Adult cardiomyocytes stained as in (e) were assessed for the ratio of mononucleated to multinucleated cardiomyocytes. (At least 150 cells were analyzed from n=3 mice per group).

FIG. 18 g, Representative images of picrosirius red-stained histological sections of sham- or TAC-operated Mlc2v-cre⁺ and Mlc2v-cre⁻ mice transduced with AAV9-fl/fl-shMthfd1l. Scale bar is 500 μm.

FIG. 18 h, Relative expression of Col1a1, Col3a1 and TGFb1 mRNA in left ventricular lysates of C57BL/6J in sham or TAC-operated Mlc2v-cre⁺ and Mlc2v⁻ cre mice injected with AAV9-fl/fl-shMthfd1l viruses. Data is normalized to sham-operated Mlc2v⁻ cre mice (set as 1.0). (n=4 for sham Mlc2v-cre-, n=5 for sham Mlc2v-cre⁺, n=8 for TAC Mlc2v-cre⁺ and n=7 for TAC Mlc2v-cre⁺ mice; shown is mean±SEM; *, % p<0.05; one-way ANOVA followed by Bonferroni correction).

FIG. 18 i, Left ventricular sections from Mlc2v-cre⁺ and Mlc2v-cre⁻ mice co-transduced with AAV9-fl/fl-shAtp5a1 and AAV9-fl/fl-shMthfd1l were stained with H&E and imaged by light microscopy 11 weeks post AAV9 injection. (3 sections/heart with 3-5 fields/section were surveyed with representative fields shown. In total, 3 hearts were surveyed per group). Scale bar is 200 μm.

FIG. 18 j, Cardiomyocytes were isolated from Mlc2v-cre⁺ and Mlc2v⁻ cre mice co-transduced with AAV9-fl/fl-shAtp5a1 and AAV9-fl/fl-shMthfd1l viruses and stained for DAPI and α-actinin. Cells were imaged by confocal microscopy and a representative z-stack image from 3 mice per group is shown. Scale bar is 20 μm.

FIG. 18 k, Adult cardiomyocytes stained as in (n) were assessed for the ratio of mononucleated to multinucleated cardiomyocytes. (At least 150 cells were analyzed from n=3 mice per group).

FIG. 18 l, LVID;s in Mlc2v-cre⁺ and Mlc2v⁻ cre mice co-transduced with AAV9-fl/fl-shAtp5a1 and AAV9-fl/fl-shMthfd1l 11 weeks post AAV9 injection. (n=6 mice per group; shown is mean±SEM).

FIG. 18 m-o, Relative expression of Nppa and Nppb (m), Atp5a1 (n), and Mthfd1l mRNA (o) from ventricular lysates obtained from Mlc2v-cre⁺ and Mlc2v-cre⁻ mice co-transduced with AAV9-fl/fl-shAtp5a1 and AAV9-fl/fl-shMthfd1l 11 weeks post AAV9 injection. Data is normalized to Mlc2v⁻ cre mice (set as 1.0). (n=6 mice per group; shown is mean±SEM; *** p<0.001; two-tailed unpaired t-test).

FIG. 18 p, Representative images of picrosirius red-stained histological sections of Mlc2v-cre⁺ and Mlc2v⁻ cre mice co-transduced with AAV9-fl/fl-shAtp5a1 and AAV9-fl/fl-shMthfd1l viruses. Scale bar is 500 μm.

FIG. 18 q, Relative expression of Col1a1, Col3a1 and TGFb1 mRNA from left ventricular lysates obtained from Mlc2v-cre⁺ and Mlc2v⁻ cre mice co-transduced with AAV9-fl/fl-shAtp5a1 and AAV9-fl/fl-shMthfd1l viruses. Data is normalized to Mlc2v⁻ cre mice (set as 1.0). (n=6 mice per group; shown is mean±SEM; * p<0.05; two-tailed unpaired t-test).

FIG. 19 a,b, Relative expression of Ccnd1 (a) and Ccne1 (b) mRNA in NRCs treated as indicated. Data is normalized to control infected cells transfected with non-silencing siRNA (set as 1.0). (n=3 biological replicates per group; shown is mean±SD; * p<0.05; ** p<0.01 one-way ANOVA and Bonferroni correction).

FIG. 19 c, NRCs transduced with lentivirus expressing empty vector control or HIF1αΔODD and transfected as indicated were stained for DAPI, Ki67 and α-actinin, and imaged by confocal microscopy. An average of 4 fields per condition and experiment were imaged and representative fields of three independent experiments are shown. Scale bar is 50 μm.

FIG. 19 d,e, Quantification of the percentage of multinucleated (d) and Ki67-positive (e) cells from NRCs treated as in (c) (n=3 independent experiments with at least 100 cells analyzed per experiment and condition; shown is mean±SD; *, % p<0.05; one-way ANOVA and Bonferroni correction).

FIG. 19 f, NRCs treated as in (c) were assessed for cell size using ImageJ. (n=3 independent experiments with at least 100 cells analyzed per experiment and condition; shown is mean±SD; *, % p<0.05; one-way ANOVA and Bonferroni correction).

FIG. 19 g, Relative expression of Cdkn1b mRNA in NRCs treated as indicated. Data is normalized to cells transfected with non-silencing siRNA (set as 1.0). (n=3 biological replicates per group; shown is mean±SD; ** p<0.01; two-tailed unpaired t-test).

FIG. 19 h, NRCs transfected with non-silencing or Cdkn1b siRNAs were stained for DAPI, Ki67 and α-actinin, and imaged by confocal microscopy. An average of 4 fields per condition and experiment were imaged and representative fields of three independent experiments are shown. Scale bar is 50 μm.

FIG. 19 i,j, Quantification of the percentage of multinucleated (i) and Ki67-positive (j) cells from NRCs treated as in (h) (n=3 independent experiments with at least 100 cells analyzed per experiment and condition; shown is mean±SD; * p<0.05; two-tailed unpaired t-test).

FIG. 19 k, NRCs treated as in (h) were assessed for cell size using ImageJ. (n=3 independent experiments with at least 100 cells analyzed per experiment and condition; shown is mean±SD; * p<0.05; two-tailed unpaired t-test).

FIG. 20 a, Relative expression of Aldh1l1 mRNA in left ventricular biopsies of C57BL/6J mice subjected to either sham or TAC surgery by qPCR. Data is shown as 2\hat{ }-dCt relative to Hprt1. (n=5 for sham, n=7 TAC; shown is mean±SEM; ** p<0.01; two-tailed unpaired t-test).

FIG. 20 b, Relative expression of ALDH1L1 mRNA in left ventricular biopsies from hypertrophic cardiomyopathy (HCM) and aortic stenosis (AS) patients versus healthy controls. Data is shown as 2\hat{ }-dCt relative to HPRT1. (n=4 for healthy controls; n=10 for AS and n=11 for HCM; shown is mean±SEM).

FIG. 20 c, NADPH quantification in NRCs treated as indicated. All values were normalised to the protein content. (n=3 biological replicates per group; shown is mean±SD).

FIG. 20 d, Immunoblot of left ventricular lysates from HCM and aortic stenosis patients and healthy controls using a methylated lysine antibody. Loading is normalized to cardiac actin.

FIG. 20 e-j, Immunoblot of left ventricular lysates from mice treated as indicated using a methylated lysine antibody. Loading is normalized to cardiac actin.

TABLE 1
Echocardiographic analysis of Mlc2v-cre−/-cre+
mice injected with AAV9-fl/fl-shAtp5a1 viruses

AAV9-fl/fl-shAtp5a1

| | Mlc2v-cre− | Mlc2v-cre+ |
|---|---|---|
| n | 8 | 9 |
| Heart rate (bpm) | 472.25 ± 36.12 | 498.53 ± 46.53 |
| IVS; d (mm) | 0.74 ± 0.03 | 0.68 ± 0.05 |
| IVS; s (mm) | 1.02 ± 0.06 | 0.90 ± 0.06 |
| LVID; d (mm) | 3.68 ± 0.16 | 3.92 ± 0.11 |
| LVID; s (mm) | 2.30 ± 0.10 | 2.96 ± 0.08 |
| LVPW; d (mm) | 0.72 ± 0.03 | 0.74 ± 0.06 |
| LVPW; s (mm) | 1.05 ± 0.03 | 1.06 ± 0.04 |
| LVID Trace (CO) (ml/min) | 16.89 ± 2.19 | 16.89 ± 1.21 |
| LVID Trace (SV) (μl) | 35.85 ± 4.97 | 34.02 ± 2.86 |
| % FS | 32.51 ± 2.92 | 24.55 ± 1.96** |
| % EF | 65.45 ± 3.83 | 49.27 ± 3.17** |
| LV Mass (mg) | 90.45 ± 8.30 | 88.68 ± 7.40 |
| LV Vol; d (μl) | 57.53 ± 5.63 | 66.75 ± 4.49 |
| LV Vol; s (μl) | 22.42 ± 1.91 | 33.81 ± 2.29* |
| LVW/BW (echo) | 3.18 ± 0.26 | 3.57 ± 0.27 |
| HW/BW (post sacrifice) | 3.48 ± 0.08 | 4.13 ± 0.12** |

IVS, intraventricular septum thickness at diastole (d) and systole (s); LVID, left ventricular internal diameter at diastole (d) and systole (s); LVPW, left ventricular posterior wall thickness at diastole (d) and systole (s); FS, fractional shortening; EF, ejection fraction; LVW/BW, left ventricular weight/body weight; HW/BW, heart weight/body weight.
Values shown are mean ± s.e.m.;
*P < 0.05;
**P < 0.01; two-tailed unpaired t-test.

TABLE 2
Echocardiographic analysis of Mlc2v-cre−/-cre+
mice injected with AAV9-fl/fl-mir27b viruses

AAV9-fl/fl-mir27b

| | Mlc2v-cre− | Mlc2v-cre+ |
|---|---|---|
| n | 6 | 8 |
| Heart rate (bpm) | 494.81 ± 22.74 | 446.51 ± 11.06 |
| IVS; d (mm) | 0.71 ± 0.02 | 0.73 ± 0.01 |
| IVS; s (mm) | 1.00 ± 0.03 | 0.98 ± 0.01 |
| LVID; d (mm) | 3.56 ± 0.04 | 4.00 ± 0.08* |
| LVID; s (mm) | 2.33 ± 0.06 | 2.90 ± 0.07* |
| LVPW; d (mm) | 0.71 ± 0.02 | 0.71 ± 0.01 |
| LVPW; s (mm) | 1.05 ± 0.02 | 0.95 ± 0.01 |
| LVID Trace (CO) (ml/min) | 17.17 ± 1.43 | 17.41 ± 0.85 |
| LVID Trace (SV) (μl) | 34.33 ± 1.40 | 38.99 ± 1.55 |
| % FS | 34.54 ± 1.30 | 27.58 ± 0.62* |
| % EF | 64.49 ± 1.72 | 52.59 ± 1.01* |
| LV Mass (mg) | 82.89 ± 2.64 | 104.15 ± 3.83* |
| LV Vol; d (μl) | 53.10 ± 1.49 | 72.25 ± 3.32** |
| LV Vol; s (μl) | 18.90 ± 1.21 | 34.39 ± 2.00** |
| LVW/BW (echo) | 2.75 ± 0.05 | 3.18 ± 0.12* |
| HW/BW (post sacrifice) | 3.59 ± 0.07 | 3.98 ± 0.13* |

IVS, intraventricular septum thickness at diastole (d) and systole (s); LVID, left ventricular internal diameter at diastole (d) and systole (s); LVPW, left ventricular posterior wall thickness at diastole (d) and systole (s); FS, fractional shortening; EF, ejection fraction; LVW/BW, left ventricular weight/body weight; HW/BW, heart weight/body weight.
Values shown are mean ± s.e.m.;
*P < 0.05;
**P < 0.01; two-tailed unpaired t-test.

TABLE 3
Echocardiographic analysis of sham- or TAC operated mice injected with scrLNA or miR27b-5p LNAs

| days post surgery | 0 | 14 | 28 | 42 | 63 |
|---|---|---|---|---|---|
| | scrLNA sham | | | | |
| n | 5 | 5 | 5 | 5 | 5 |
| LVID; d (mm) | 3.78 ± 0.13 | 3.53 ± 0.06 | 3.43 ± 0.12 | 3.28 ± 0.08 | 3.37 ± 0.07 |
| LVID; s (mm) | 2.62 ± 0.16 | 2.24 ± 0.09 | 1.99 ± 0.19 | 1.88 ± 0.10 | 1.98 ± 0.11 |
| LVPW; d (mm) | 0.72 ± 0.02 | 0.75 ± 0.03 | 0.74 ± 0.02 | 0.76 ± 0.01 | 0.78 ± 0.03 |
| LVPW; s (mm) | 0.98 ± 0.03 | 1.08 ± 0.05 | 1.13 ± 0.05 | 1.12 ± 0.05 | 1.16 ± 0.05 |
| % FS | 30.73 ± 2.20 | 36.68 ± 1.70 | 42.28 ± 3.57 | 42.68 ± 2.44 | 41.53 ± 2.39 |
| % EF | 58.78 ± 3.25 | 65.22 ± 0.32 | 73.64 ± 4.59 | 74.63 ± 2.68 | 73.3 ± 2.68 |
| LVW/BW echo | 3.46 ± 0.18 | 3.42 ± 0.11 | 3.26 ± 0.20 | 2.79 ± 0.16 | 2.88 ± 0.11 |
| Aortic velocity (mm/sec) | 848 ± 71 | 945 ± 56 | 1008 ± 87 | 939 ± 102 | 955 ± 36 |

HW/BW (post sacrifice) 3.98 ± 0.03

| | miR27b-5p LNA sham | | | | |
|---|---|---|---|---|---|
| n | 5 | 5 | 5 | 5 | 5 |
| LVID; d (mm) | 3.66 ± 0.27 | 3.40 ± 0.06 | 3.38 ± 0.13 | 3.28 ± 0.10 | 3.54 ± 0.12 |
| LVID; s (mm) | 2.53 ± 0.27 | 2.05 ± 0.05 | 1.99 ± 0.12 | 1.93 ± 0.07 | 2.18 ± 0.09 |
| LVPW; d (mm) | 0.70 ± 0.02 | 0.76 ± 0.02 | 0.78 ± 0.01 | 0.79 ± 0.00 | 0.79 ± 0.03 |
| LVPW; s (mm) | 0.97 ± 0.04 | 1.12 ± 0.04 | 1.20 ± 0.02 | 1.18 ± 0.02 | 1.11 ± 0.03 |
| % FS | 31.50 ± 4.15 | 39.68 ± 1.54 | 41.30 ± 2.01 | 41.12 ± 0.79 | 38.31 ± 1.58 |
| % EF | 59.31 ± 5.93 | 71.25± 1.81 | 73.09 ± 2.36 | 73.14 ± 0.96 | 69.39 ± 1.95 |
| LVW/BW echo | 3.45 ± 0.18 | 3.20 ± 0.16 | 3.15 ± 0.18 | 3.12 ± 0.16 | 3.28 ± 0.21 |
| Aortic velocity (mm/sec) | 799 ± 19 | 864 ± 84 | 802 ± 42 | 960 ± 45 | 876 ± 71 |

HW/BW (post sacrifice) 4.20 ± 0.18

| | scrLNA TAC | | | | |
|---|---|---|---|---|---|
| n | 7 | 7 | 7 | 7 | 7 |
| LVID; d (mm) | 3.79 ± 0.07 | 3.53 ± 0.09 | 3.72 ± 0.12 | 4.01 ± 0.15* | 4.24 ± 0.18*** |
| LVID; s (mm) | 2.80 ± 0.08 | 2.54 ± 0.10 | 2.71 ± 0.12 | 3.33 ± 0.15* | 4.21 ± 0.25** |
| LVPW; d (mm) | 0.71 ± 0.02 | 0.97 ± 0.04* | 1.01 ± 0.04* | 1.05 ± 0.05 | 1.13 ± 0.07** |
| LVPW; s (mm) | 0.92 ± 0.02 | 1.30 ± 0.05** | 1.36 ± 0.06 | 1.29 ± 0.04 | 1.38 ± 0.05 |
| % FS | 28.60 ± 1.30 | 25.78 ± 0.33 | 27.09 ± 2.01 | 19.29 ± 1.25 | 17.17 ± 2.12** |
| % EF | 55.90 ± 2.10 | 51.45 ± 3.18 | 53.23 ± 3.19 | 37.32 ± 2.95 | 36.02 ± 4.13** |
| LVW/BW echo | 3.71 ± 0.18 | 4.80 ± 0.16* | 5.27 ± 0.22* | 5.74 ± 0.45 | 7.37 ± 0.95*** |
| Aortic velocity (mm/sec) | 699 ± 29 | 4590 ± 182*** | 4612 ± 183* | 4697 ± 132*** | 4964 ± 357*** |

HW/BW (post sacrifice) 7.02 ± 0.47

| | miR27b-5p LNA TAC | | | | |
|---|---|---|---|---|---|
| n | 8 | 8 | 8 | 8 | 8 |
| LVID; d (mm) | 3.93 ± 0.07 | 2.98 ± 0.14* | 3.59 ± 0.17 | 3.91 ± 0.21 | 3.83 ± 0.25 |
| LVID; s (mm) | 2.76 ± 0.08 | 2.70 ± 0.20 | 2.57 ± 0.22 | 3.05 ± 0.33 | 2.96 ± 0.44 |
| LVPW; d (mm) | 0.72 ± 0.01 | 1.07 ± 0.04** | 0.98 ± 0.04 | 1.02 ± 0.06* | 0.93 ± 0.04* |
| LVPW; s (mm) | 0.97 ± 0.02 | 1.47 ± 0.05** | 1.32 ± 0.06* | 1.25 ± 0.04 | 1.29 ± 0.05* |
| % FS | 29.26 ± 1.64 | 27.38 ± 2.40 | 28.83 ± 2.96 | 15.28 ± 0.97* | 27.52 ± 1.81 |
| % EF | 56.56 ± 2.53 | 52.37 ± 4.70 | 55.92 ± 4.58 | 32.62 ± 2.49** | 54.79 ± 2.28 |
| LVW/BW echo | 3.85 ± 0.20 | 4.22 ± 0.16 | 4.98 ± 0.29* | 5.73 ± 0.65* | 5.62 ± 0.65* |
| Aortic velocity (mm/sec) | 775 ± 40 | 4638 ± 78*** | 4301 ± 259*** | 4544 ± 364*** | 4843 ± 234*** |

HW/BW (post sacrifice) 6.06 ± 0.21
LVID, left ventricular internal diameter at diastole (d) and systole (s); LVPW, left ventricular posterior wall thickness at diastole (d) and systole (s); FS, fractional shortening; EF, ejection fraction; LVW/BW, left ventricular weight/body weight; HW/BW, heart weight/body weight.
Values shown are mean ± s.e.m.;
*P < 0.05;
**P < 0.01;
TAC scrLNAvs. sham scrLNA; miR27b-5p LNA sham vs. miR27b-5p LNA TAC; two-tailed unpaired t-test.

TABLE 4
Echocardiographic analysis of sham- or TAC operated Mlc2v-cre−/-cre+
mice injected with AAV9-fl/fl-shMthfd1l viruses

| | AAV9-fl/fl-shMthfd1l | | | |
|---|---|---|---|---|
| | sham | | TAC | |
| | Mlc2v-cre− | Mlc2v-cre+ | Mlc2v-cre− | Mlc2v-cre+ |
| n | 4 | 5 | 8 | 7 |
| Heart rate (bpm) | 512.91 ± 23.90 | 486.51 ± 8.27 | 583.57 ± 82.17 | 561.55 ± 19.39 |
| IVS; d (mm) | 0.74 ± 0.01 | 0.74 ± 0.01 | 1.11 ± 0.04 | 0.91 ± 0.01* |
| IVS; s (mm) | 0.98 ± 0.02 | 0.96 ± 0.01 | 1.39 ± 0.05 | 1.11 ± 0.03* |
| LVID; d (mm) | 3.84 ± 0.11 | 3.70 ± 0.16 | 4.26 ± 0.28 | 3.56 ± 0.13* |
| LVID; s (mm) | 2.74 ± 0.13 | 2.57 ± 0.17 | 3.58 ± 0.41 | 2.36 ± 0.39* |
| LVPW; d (mm) | 0.71 ± 0.01 | 0.75 ± 0.01 | 0.99 ± 0.07 | 0.94 ± 0.04 |
| LVPW; s (mm) | 0.96 ± 0.03 | 1.03 ± 0.02 | 1.24 ± 0.15 | 1.15 ± 0.10 |
| % EF | 49.64 ± 2.62 | 57.81 ± 3.29 | 37.72 ± 7.75 | 58.03 ± 7.27* |
| LVID Trace (CO) (ml/min) | 17.25 ± 1.49 | 16.11 ± 0.47 | 14.98 ± 1.16 | 16.56 ± 4.11 |
| LVID Trace (SV) (μl) | 33.42 ± 1.74 | 33.25 ± 1.01 | 27.81 ± 1.78 | 31.36 ± 4.95 |
| LV mass (mg) | 86.08 ± 5.84 | 95.33 ± 3.15 | 161.33 ± 11.27 | 82.91 ± 9.45*** |
| LV Vol; d (μl) | 55.57 ± 4.49 | 63.98 ± 2.61 | 66.96 ± 10.88 | 48.82 ± 8.26* |
| LV Vol; s (μl) | 23.80 ± 4.83 | 32.34 ± 1.32 | 39.39 ± 11.00 | 17.72 ± 2.10* |
| LVW/BW (echo) | 3.86 ± 0.54 | 3.42 ± 0.19 | 6.32 ± 0.16 | 4.12 ± 0.19** |
| HW/BW (post sacrifice) | 4.07 ± 0.41 | 4.01 ± 0.29 | 6.53 ± 0.53 | 4.91 ± 0.03* |
| Aortic Vel. (mm/s) | 881 ± 68 | 864 ± 207 | 4552 ± 53 | 4725 ± 70 |

IVS, intraventricular septum thickness at diastole (d) and systole (s); LVID, left ventricular internal diameter at diastole (d) and systole (s); LVPW, left ventricular posterior wall thickness at diastole (d) and systole (s); EF, ejection fraction; LVW/BW, left ventricular weight/body weight; HW/BW, heart weight/body weight.
Values shown are mean ± s.e.m.;
*P < 0.05;
**P < 0.01 TACMlc2v-cre−vs. TACMlc2v-cre+; two-tailed unpaired t-test.

TABLE 5
Echocardiographic analysis of Mlc2v-cre−/-cre+ mice co-injected
with AAV9-fl/fl-shAtp5a1 and AAV9-fl/fl-shMthfd1l viruses

AAV9-fl/fl-shAtp5a1/shMthfd1l

| | Mlc2v-cre− | Mlc2v-cre+ |
|---|---|---|
| n | 6 | 6 |
| Heart rate (bpm) | 499.60 ± 11.38 | 511.23 ± 9.01 |
| IVS; d (mm) | 0.72 ± 0.01 | 0.73 ± 0.02 |
| IVS; s (mm) | 0.97 ± 0.03 | 0.99 ± 0.04 |
| LVID; d (mm) | 3.87 ± 0.11 | 4.09 ± 0.12 |
| LVID; s (mm) | 2.82 ± 0.18 | 2.86 ± 0.17 |
| LVPW; d (mm) | 0.74 ± 0.04 | 1.01 ± 0.03* |
| LVPW; s (mm) | 0.98 ± 0.04 | 1.06 ± 0.04 |
| LVID Trace (CO) (ml/min) | 18.78 ± 1.36 | 20.03 ± 1.56 |
| LVID Trace (SV) (μl) | 37.49 ± 2.24 | 39.00 ± 2 34 |
| % FS | 29.28 ± 2.51 | 27.42 ± 2.83 |
| % EF | 56.35 ± 4.04 | 53.20 ± 4.44 |
| LV Mass (mg) | 97.47 ± 5.73 | 107.89 ± 4.88 |
| LV Vol; d (μl) | 65.08 ± 4.61 | 74.42 ± 5.12 |
| LV Vol; s (μl) | 29.04 ± 4.41 | 35.68 ± 5.28 |
| LVW/BW (echo) | 3.66 ± 0.22 | 3.93 ± 0.21 |
| HW/BW (post sacrifice) | 4.14 ± 0.13 | 4.40 ± 0.25 |

IVS, intraventricular septum thickness at diastole (d) and systole (s); LVID, left ventricular internal diameter at diastole (d) and systole (s); LVPW, left ventricular posterior wall thickness at diastole (d) and systole (s); FS, fractional shortening; EF, ejection fraction; LVW/BW, left ventricular weight/body weight; HW/BW, heart weight/body weight.
Values shown are mean ± s.e.m.;
*P < 0.05;
**P < 0.01; two-tailed unpaired t-test.

### Examples

F₁F₀ ATP Synthase Repression Drives Cardiometabolic Endoreplication and Pathologic Growth

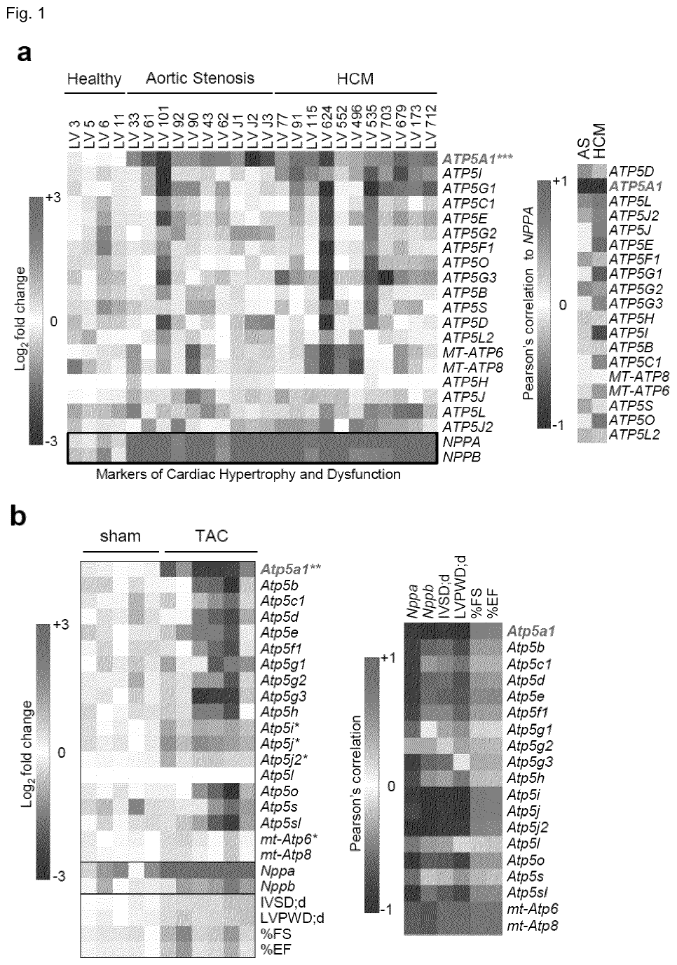

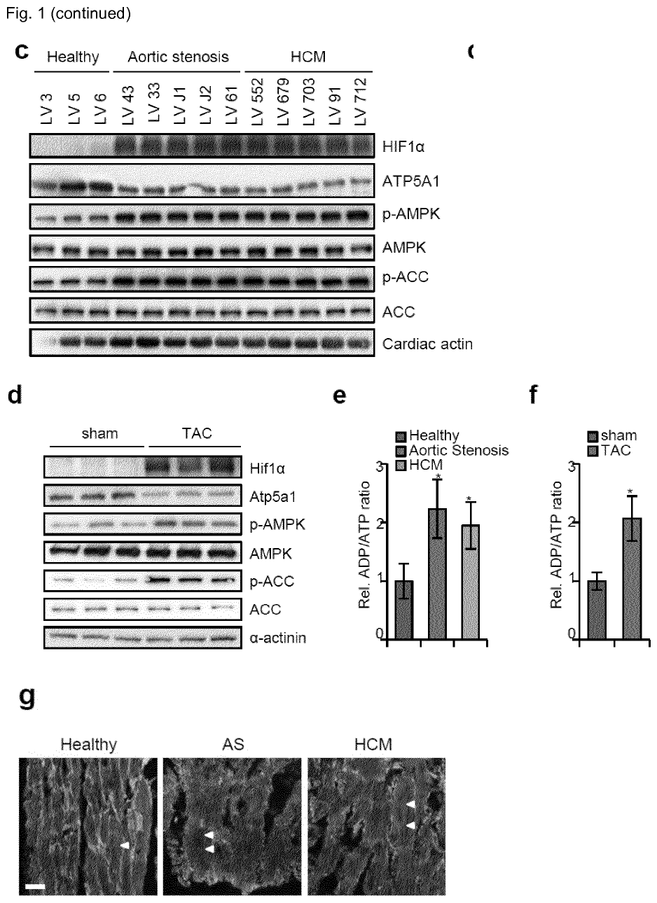

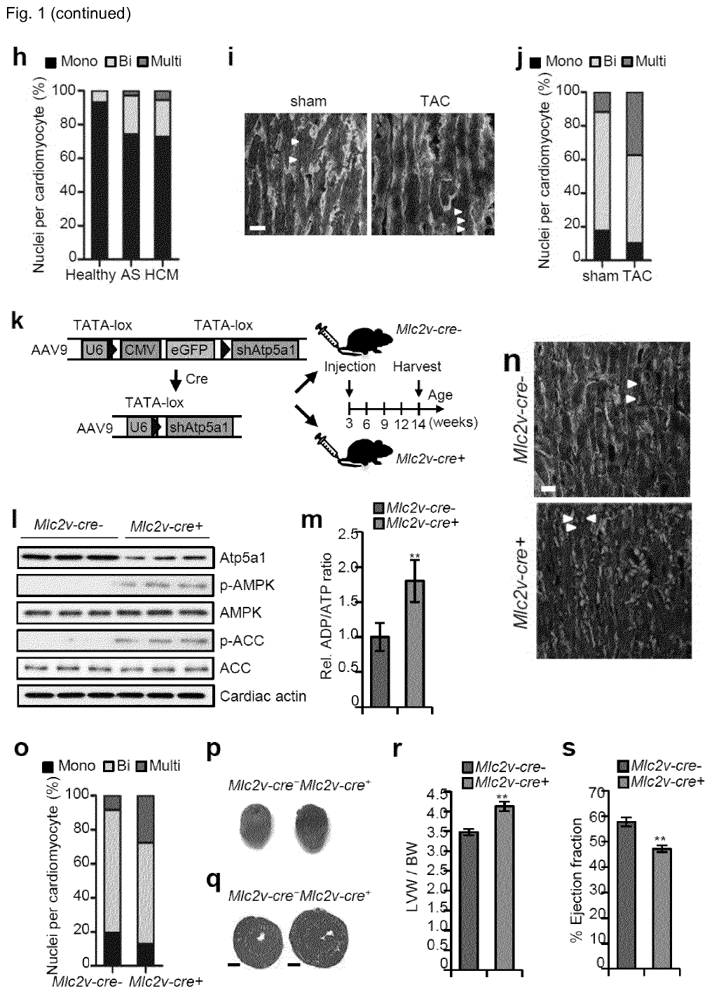

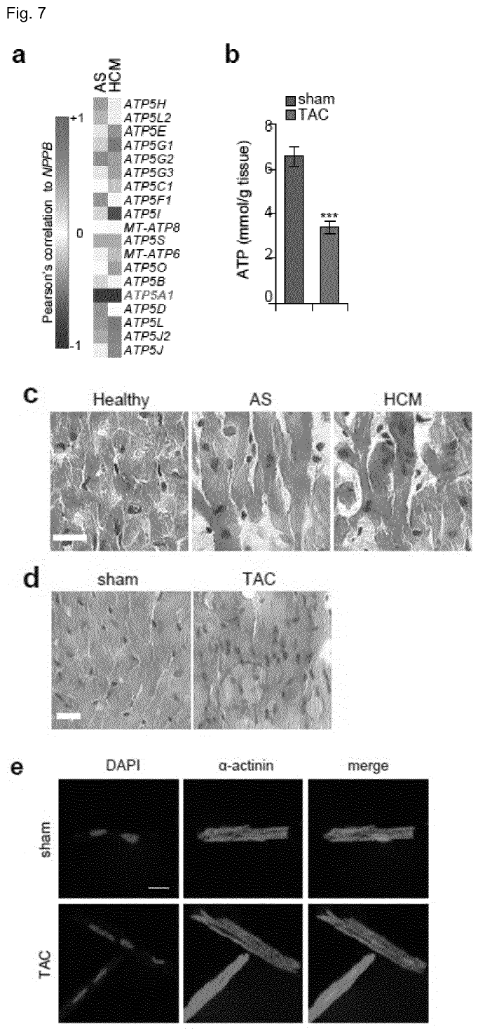

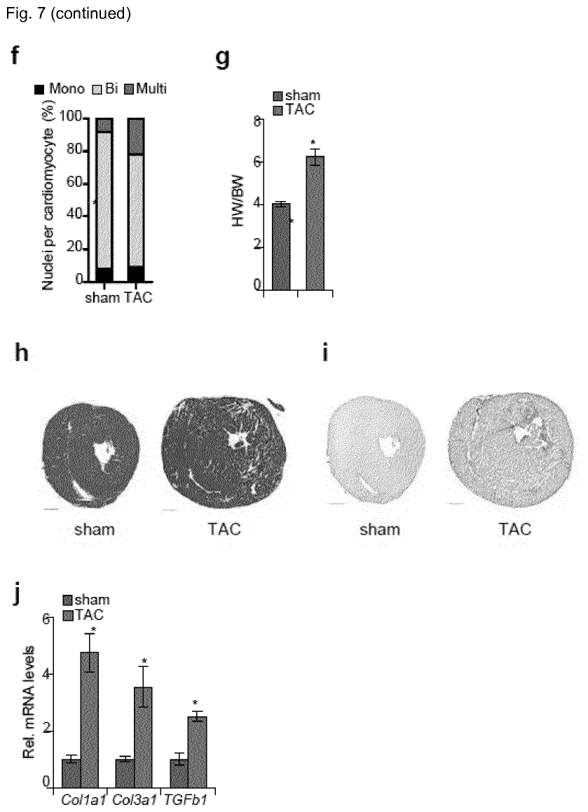

In order to identify mediators of ATP depletion in human and mouse pathologic cardiac hypertrophy we profiled mRNA levels of all subunits of the ATP synthase complex in patient biopsies of human HCM and AS, and in mice subjected to TAC (FIG. 1a,b and FIG. 7a). Among the subunits profiled, ATP5A1 mRNA and protein was consistently reduced in both human and mouse cardiac hypertrophy and correlated significantly with upregulation of the hypertrophic markers natriuretic peptide A (NPPA), natriuretic peptide B (NPPB), and echocardiographically measured disease indicators of cardiac morphology and function (FIG. 1a-d and FIG. 7a). These changes in ATP5A1 levels correlated with activation of AMP-activated protein kinase (AMPK) through increased phosphorylation at Thr172 in the diseased myocardium of both humans and mice, resulting in phosphorylation and downstream inactivation of its direct target Acetyl-CoA-Carboxylase (ACC) (FIG. 1c,d). Critically, although historically termed as being an AMP activated kinase, AMPK is in fact more sensitive and predominantly activated by ADP. In line with repressed ATP5A1 expression and AMPK activation, ADP:ATP ratios were elevated in diseased human and mouse left ventricular biopsies (FIG. 1e,f and FIG. 7b). In addition, an increased fraction of cardiomyocytes exhibiting multinucleation was observed in human left ventricular AS and HCM biopsies (FIG. 1g,h and FIG. 7c). In mice, an increase in the fraction of multinucleated cells was also observed after TAC surgery (FIG. 1i,j and FIG. 7d-f). The TAC protocol mimics human aortic stenosis through surgical constriction of the mouse aorta, resulting in increased blood velocity into the ventricle to impose a pressure overload stress leading to pathologic cardiac growth and fibrosis indicated by increased picrosirius red staining and expression of the fibrotic marker genes collagen type I alpha 1 (Col1a1), collagen type I alpha 3 (Col3a1) and transforming growth factor beta 1 (TGF1b) as encountered in human aortic stenosis (Barrick, C. J., et al., 2007, Am J Physiol Heart Circ Physiol, 292, 2119-2130). (FIG. 7g-j).

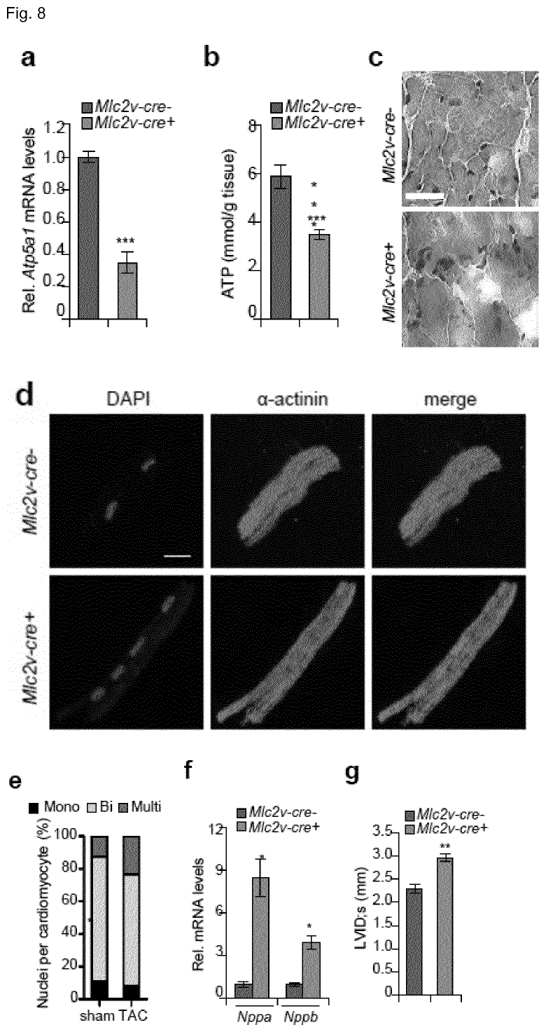

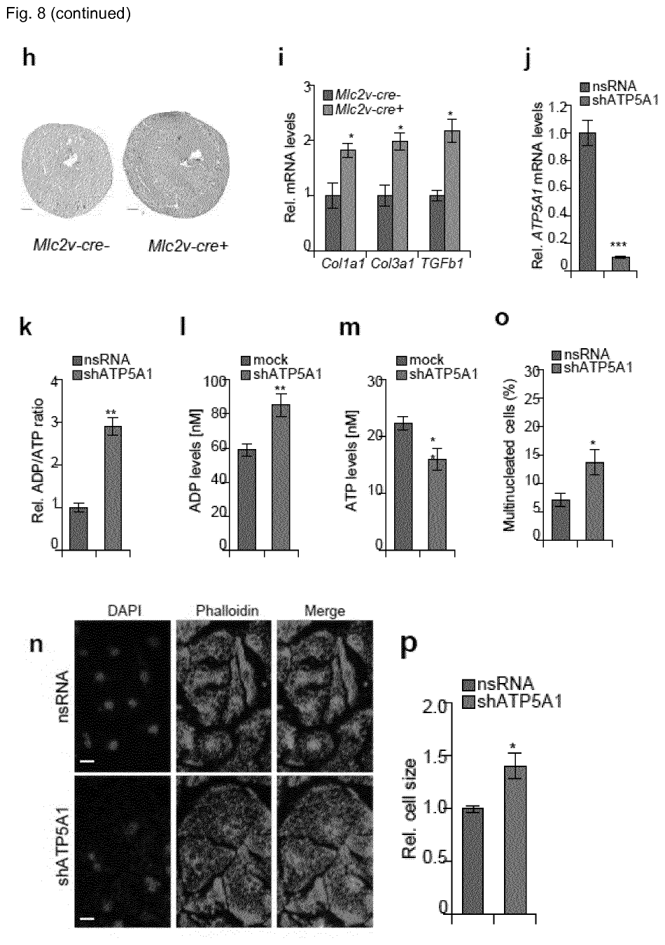

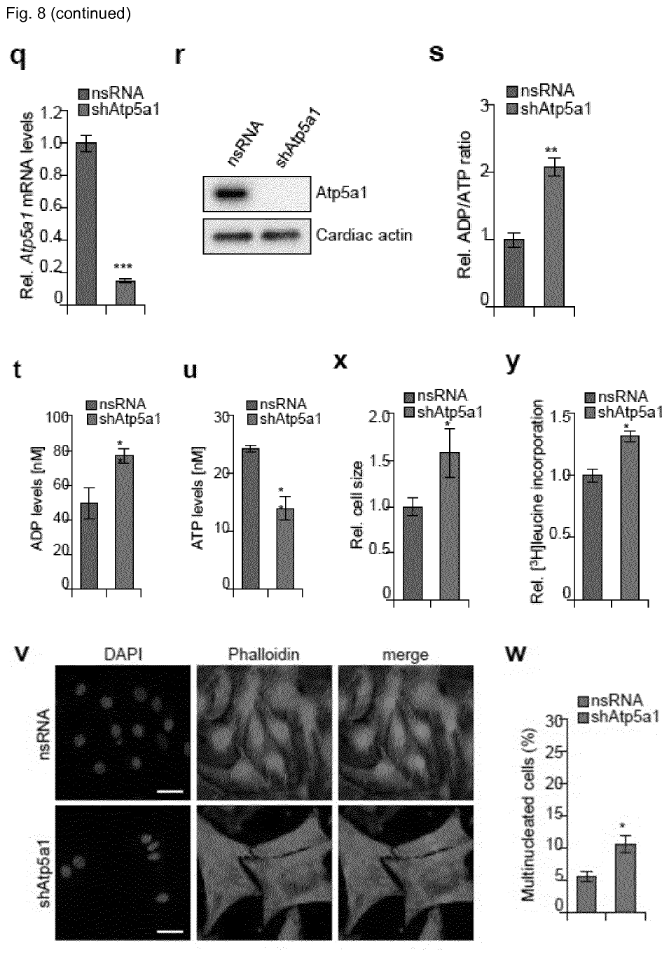

To understand the physiologic implication of ATP5A1 depletion, we utilized a novel adeno-associated virus (AAV)9-based system for in vivo ventricular-specific transgenesis (Mirtschink, P., et al., 2015, Nature, 522, 444-449). Briefly, delivery of an AAV containing TATA-lox flanked genes or short-hairpin RNAs (shRNA) imparts Cre recombinase (Cre) dependency, which upon delivery to tissue-specific Cre transgenic mice results in tissue-specific ectopic gene or shRNA expression (Sauer, B., et al., 1988, Proc Natl Acad Sci USA, 85, 5166-5170). AAV9 harboring shRNAs targeting Atp5a1 (AAV9-fl/fl-shAtp5a1) was delivered to Mlc2v-Cre transgenic mice (Chen, J., et al., 1998, Development, 125, 1943-1949). that express Cre recombinase specifically in the ventricular myocardium (FIG. 1k). AAV9-fl/fl-shAtp5a1 delivery led to repressed Atp5a1 mRNA and protein expression (FIG. 1l and FIG. 8a), with concomitant activation of Ampk and augmented phosphorylation of its downstream target Acc (FIG. 1l), increased ADP:ATP levels (FIG. 1m and FIG. 8b), cardiomyocyte multinucleation and overgrowth combined with significantly reduced cardiac contractility and fibrosis (FIG. 1n-s, FIG. 8c-i and Table 1). Next, we confirmed these findings in induced pluripotent stem cell (iPSC)-derived human cardiomyocytes and primary neonatal rat cardiomyocytes (NRC). Human and rat cardiomyocytes were stained with DAPI to visualize nuclei, and phalloidin to label filamentous actin and outline the cell surface. As noted in FIG. 8j-y, knockdown of ATP5A1 with shRNAs in human and rat heart cells resulted in increased ADP:ATP ratio (FIG. 8k-m, s-u), confirmed by elevated levels of ADP and ATP depletion (FIG. 8t, u), and cardiomyocyte multinucleation, concomitant to cell overgrowth as quantified by 2D cell size analysis and leucine incorporation, a readout for protein synthesis as an indirect measure of growth (FIG. 8n-p, v-y) (Fukuzawa, J., et al., 2000. Hypertension, 35, 1191-1196). Thus, ATP5A1 depletion is sufficient to induce endoreplication and multinucleation to drive pathologic cardiac growth and compromise cardiac function.

HIF1α regulates F₁F₀ ATP synthase activity via mir27b-5p

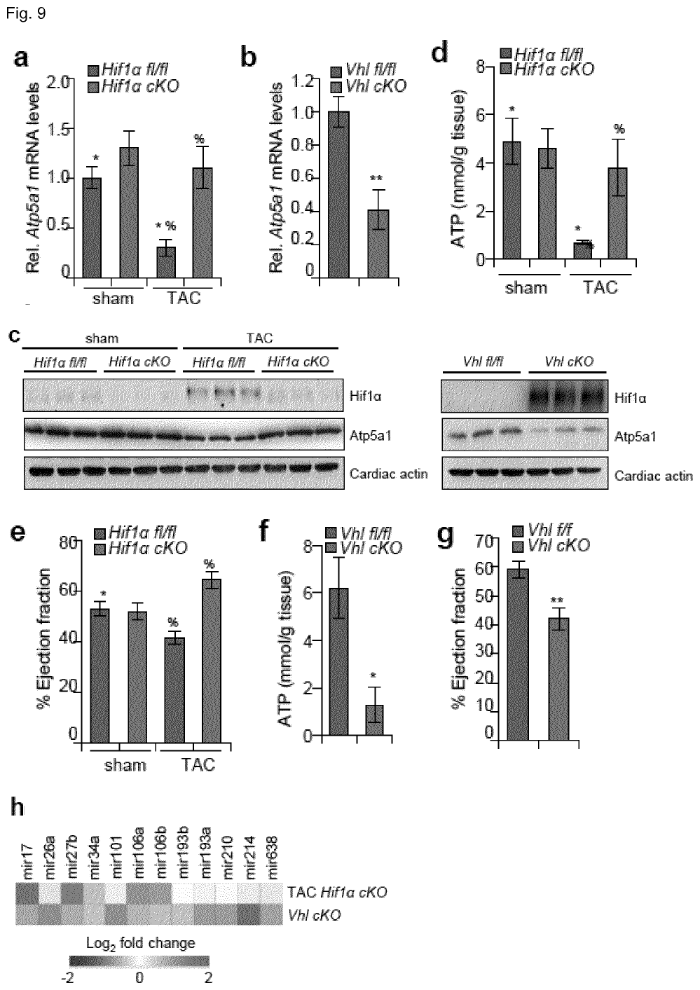

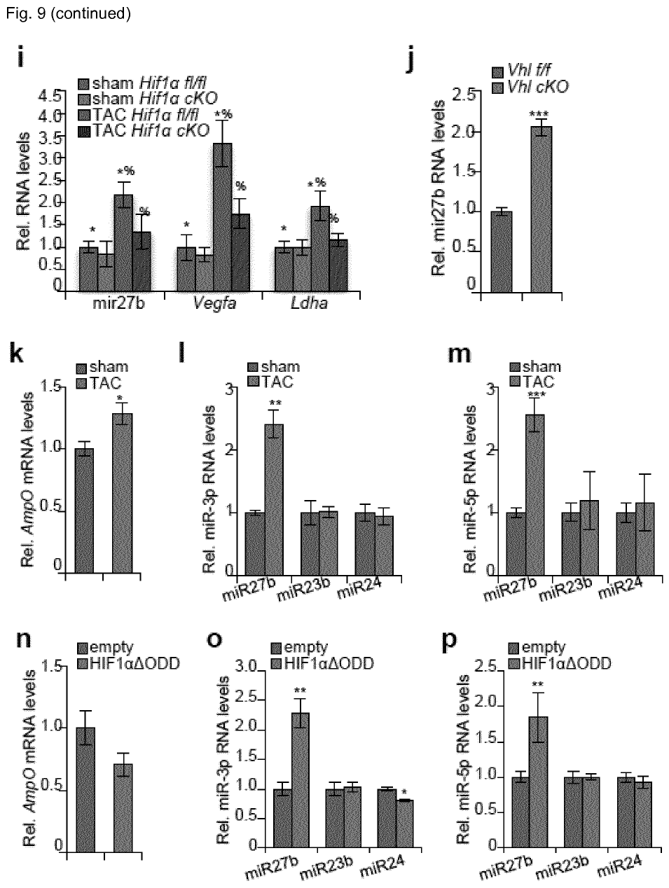

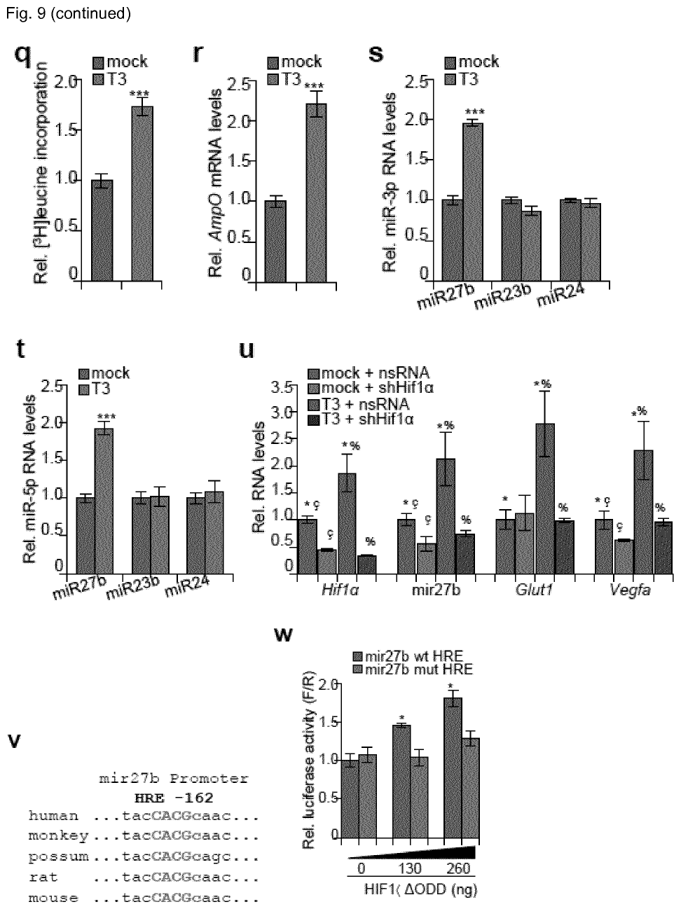

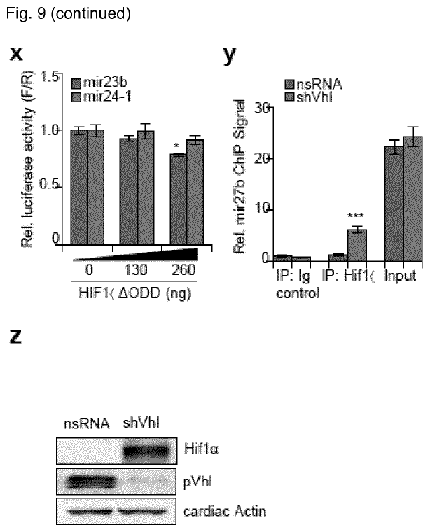

We detected inverse co-regulation of cardiac Hif1α activation with Atp5a1 mRNA and protein expression in ventricular-specific Hif1α conditional knockout (cKO) mice subjected to TAC, and in mice deficient for the von Hippel Lindau protein (Vhl) in the ventricular myocardium, leading to myocardial Hif1α accumulation, due to inhibited HIF1α degradation via its oxygen dependent subunit (FIG. 9a-c). As a consequence of the inverse relationship between HIF1α accumulation and ATP5A1 repression, ventricular-specific Hif1α cKO mice were protected from the ATP deficiency and contractile dysfunction observed in control Hif1α fl/fl littermates subjected to TAC (FIG. 9d,e), whereas Vhl cKO mice displayed low ventricular ATP levels associated with decreased systolic function (FIG. 9f,g). However, in silico and expression studies repeatedly failed to pinpoint direct Hif1α target genes capable of mediating the observed effects on Atp5a1 mRNA and ATP levels (data not shown). This led us to explore the role of Hif1α-regulated miRNAs in mediating these effects. We performed expression profiling of miRNAs from left ventricles of control and ventricular-specific Hif1α cKO mice subjected to TAC, and on ventricle-specific Vhl cKO mice. By comparing expression data within the respective groups and filtering for miRNAs containing cross-species conserved hypoxia response element (HRE) motifs, we identified several potential Hif1α-regulated miRNAs (FIG. 9h). A subset of these miRNAs have been previously linked to hypoxia and/or Hif1α dependence. Of these miRNAs, mir27b was unique by its repression in Hif1α cKO mice subjected to TAC (compared to littermate control Hif1α fl/fl TAC mice) in line with the expression pattern of established HIF1α target genes, and its upregulation in ventricular-specific Vhl cKO mice (FIG. 9i,j), suggesting potential regulation by Hif1α.

mir27b is contained within the mir23b-27b-24 cluster in intron 15 of the aminopeptidase O (AmpO) gene in humans and mice (Zhou, Q., et al., 2011, Proc Natl Acad Sci USA, 108, 8287-8292). To determine the stress- and Hif1α-dependence of mir27b transcription, we assayed expression of AmpO and all miRNAs contained within the cluster in mice subjected to TAC, and in vitro in response to expression of a constitutively active Hif1α mutant lacking the oxygen dependent degradation domain (ODD) (referred to as HIF1αΔODD) or by stimulation with the hypertrophy-inducing p-adrenergic receptor agonist triiodothyronine (T3) (FIG. 9k-t). Application of TAC in mice resulted in a slight induction of AmpO, largely unchanged expression of mature miR23b-3p/5p and miR24-3p/5p, and a ˜2.5-fold induction of miR27b-3p/5p (FIG. 9k-m).

In vitro ectopic HIF1αΔODD expression led to AmpO downregulation and unchanged levels of miR23b-3p/5p and miR24-3p/5p (FIG. 9n-p), while T3 stimulation induced AmpO expression but not that of miR23b-3p/5p or miR24-3p/5p (FIG. 9r-t). In contrast, mature miR27b-3p/5p was consistently upregulated in both in vitro models (FIG. 9p,t). These results suggest that miR27b-3p/5p is regulated independently of its host gene AmpO and its neighboring miRNAs in response to stress and Hif1a. To confirm MIR27B as a direct HIF1α target, we stimulated control non-silencing (ns) and shHif1α transduced NRCs with T3 and analyzed the expression of mir27b and established Hif1α target genes as controls (FIG. 9u). In silico analysis for cross-species conserved HREs in the mir27b promoter, corresponding to −1 kb upstream of the pre-mir27b transcriptional start site (TSS) revealed a conserved HRE motif close to the TSS in humans and mice (FIG. 9v). To assess functionality of this HRE, mir27b promoter-luciferase assays with the wildtype and HRE-mutated promoter were performed. Hif1α increased luciferase reporter expression only of the wildtype promoter but not in the HRE mutant promoter or in control cells, respectively (FIG. 9w). Promoters of the neighboring miRNAs mir23b and mir24 did not show increased promoter activity in the presence of ectopic expression of HIF1αΔODD (FIG. 9x). Moreover, Hif1α specifically associated with the mir27b promoter in native chromatin of NRC transduced with shVhl as revealed by Hif1α chromatin-immunoprecipitation (ChIP) from nuclear extracts (FIG. 9y). The efficiency of pVhl depletion by shVhl and consequent Hif1α accumulation was confirmed by immunoblotting (FIG. 9z). Taken together, these data define MIR27B as a bona fide HIF1α target miRNA.

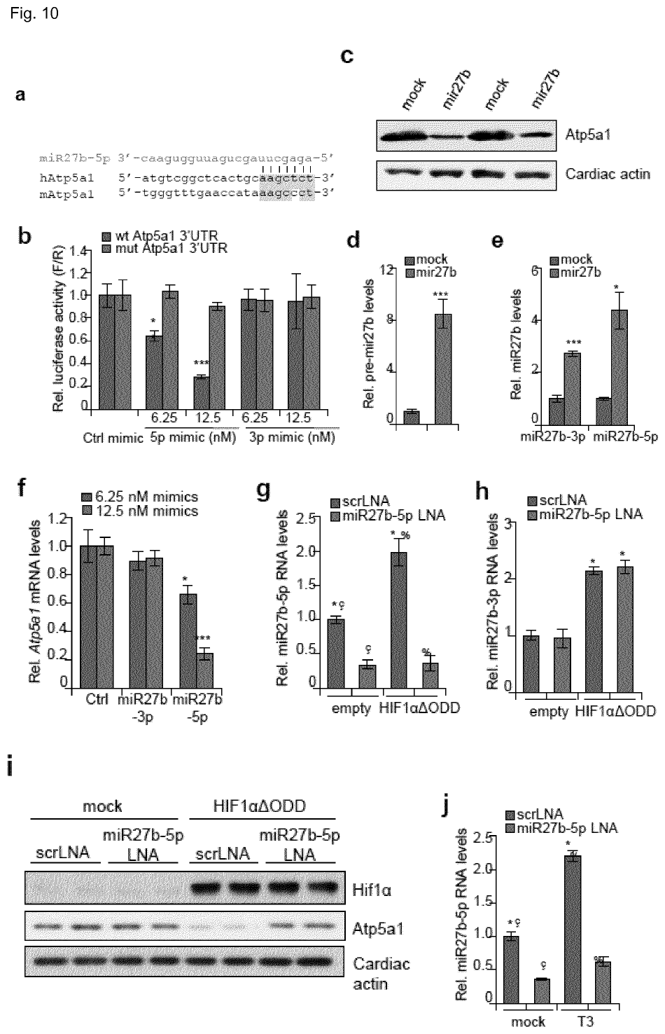

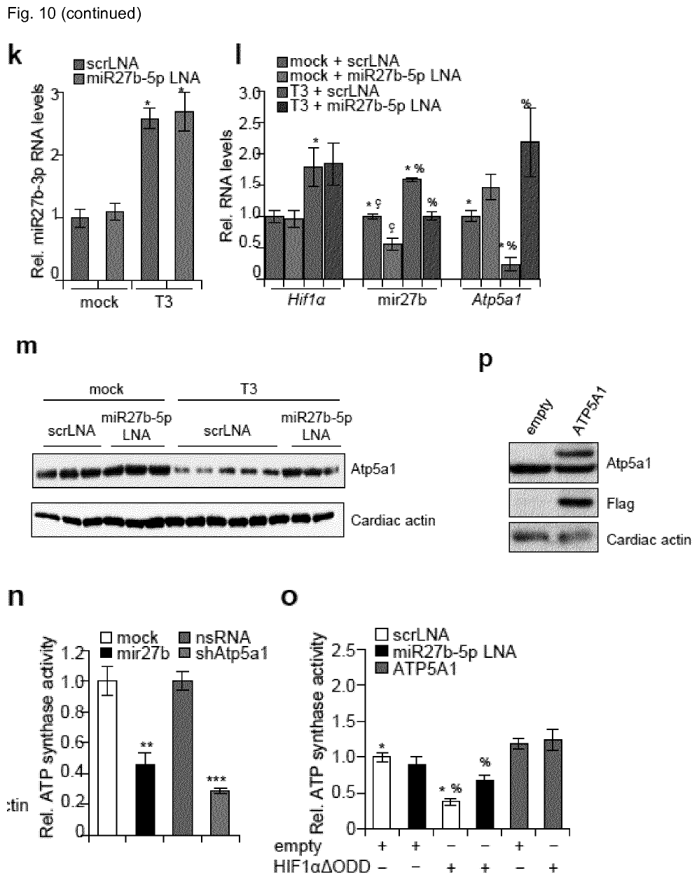

Atp5a1 harbors a miR27b-binding site in the 3′UTR, conserved between human and mice (FIG. 10a), which upon disruption by site-directed mutagenesis, results in de-repression of mir27b-mediated inhibition (FIG. 10b). In accord, ectopic mir27b expression in NRC resulted in pronounced repression of Atp5a1 mRNA and protein (FIG. 10c-e). Despite upregulation of both miR27b-3p and miR27b-5p RNA by HIF1α, miR27b-5p was unique by its capacity to inhibit wildtype Atp5a1 UTR-reporter expression (FIG. 10b). Treatment of cardiomyocytes with miR27b-5p mimics led to the dose-dependent downregulation of Atp5a1 mRNA but miR27b-3p mimics did not (FIG. 10f). These observations were recapitulated with locked nucleic acid (LNA)-mediated miR27b-5p inhibition in cardiomyocytes expressing ectopic HIF1αΔODD (FIG. 10g-i). LNAs specifically targeting miR27b-5p led to de-repression of HIF1αΔODD induced Atp5a1 downregulation (FIG. 10g-i). Similar results were observed upon T3 stimulation where Atp5a1 repression was rescued upon simultaneous treatment with miR27b-5p LNAs (FIG. 10j-m). Thus, miR27b inhibition of Atp5a1 is mediated specifically by the miR27b-5p species.

In order to determine if downregulation of Atp5a1 under these settings affected ATP synthase activity, we measured the ATP regenerating capacity of immunoprecipitated ATP synthase isolated from rat cardiomyocytes ectopically expressing mir27b or shAtp5a1; or upon ectopic HIF1αΔODD expression in combination with miR27b-5p LNAs (FIG. 10n,o). As noted, ectopic mir27b and HIF1αΔODD expression led to significant repression of ATP synthase activity, with activity rescued upon parallel treatment with miR27b-5p LNAs (FIG. 10n,o). Atp5a1 inactivation also mimicked the effects of ectopic mir27b expression, while concomitant ATP5A1 overexpression with a flag-tagged construct (FIG. 10p) rescued the diminished ATP synthase activity upon ectopic HIF1αΔODD expression (FIG. 100). Thus, ATP synthase activity is highly dependent on Atp5a1 expression levels.

miR27b-5p Inhibition Attenuates Severe Cardiac Hypertrophy and Dysfunction

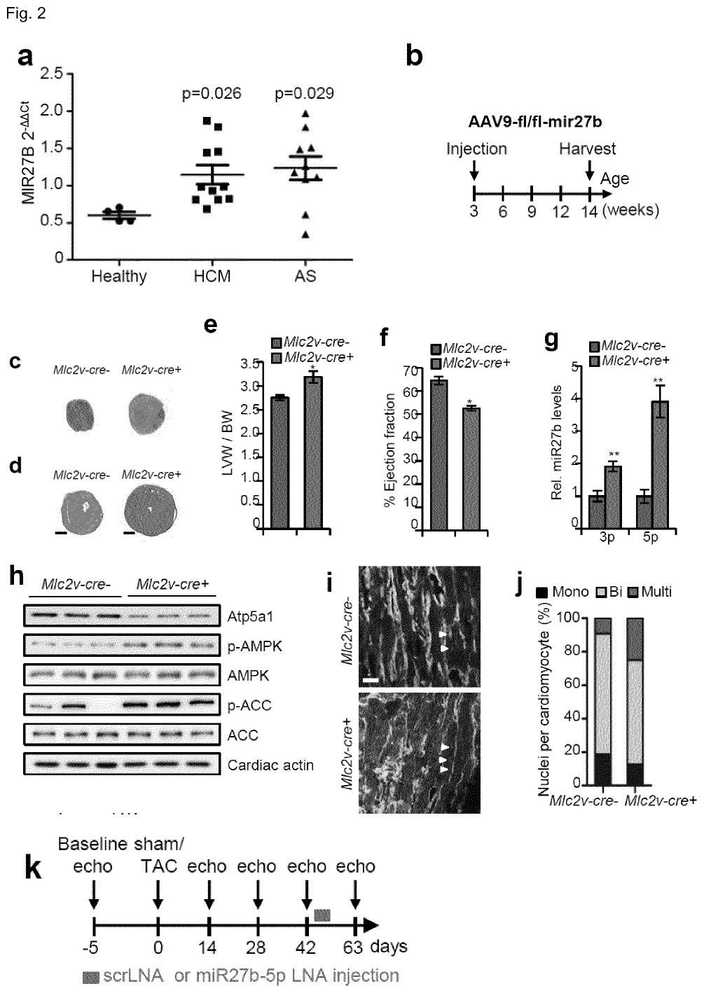

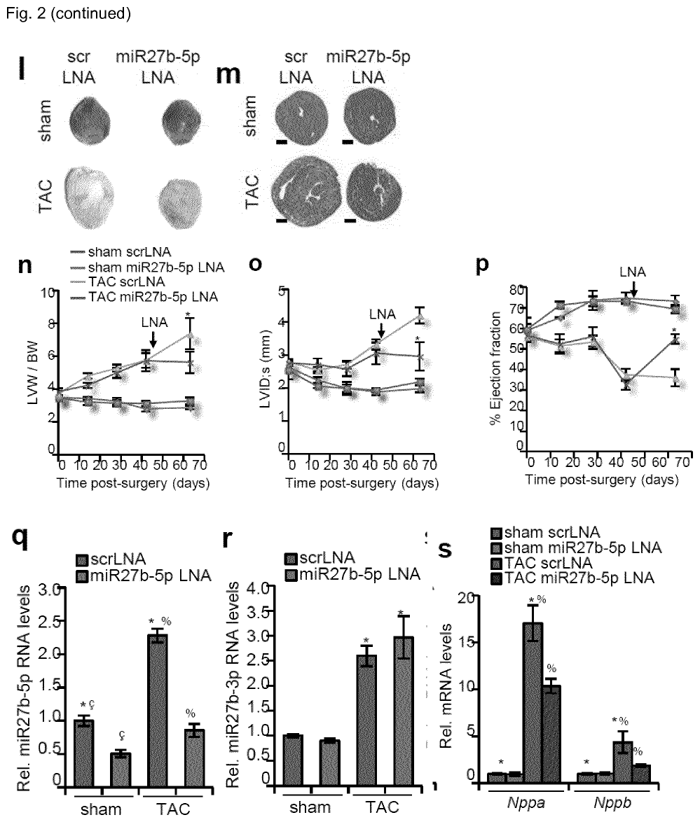

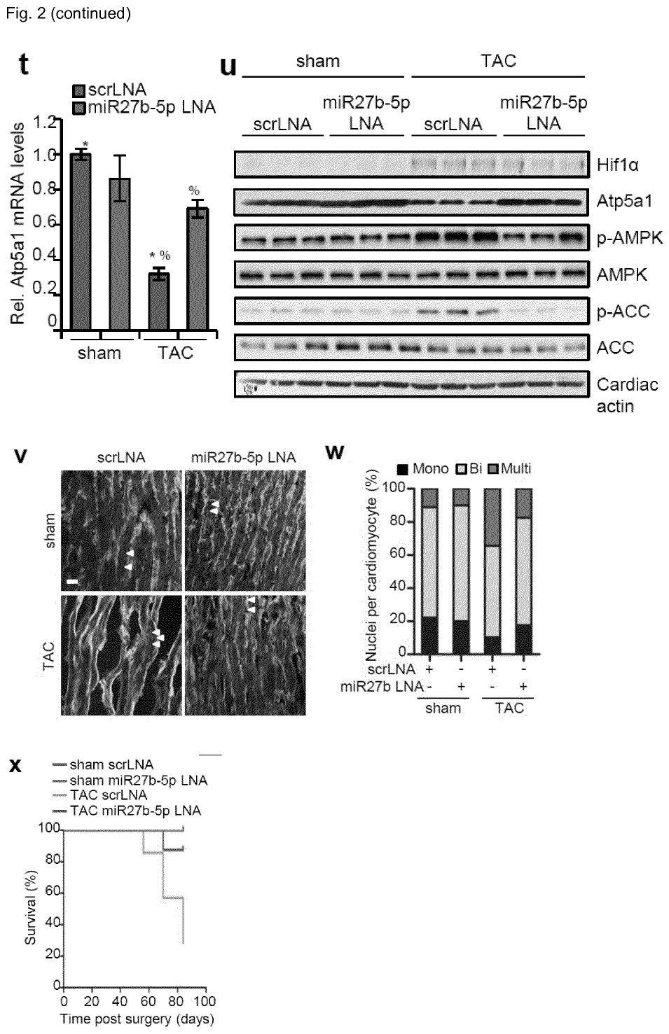

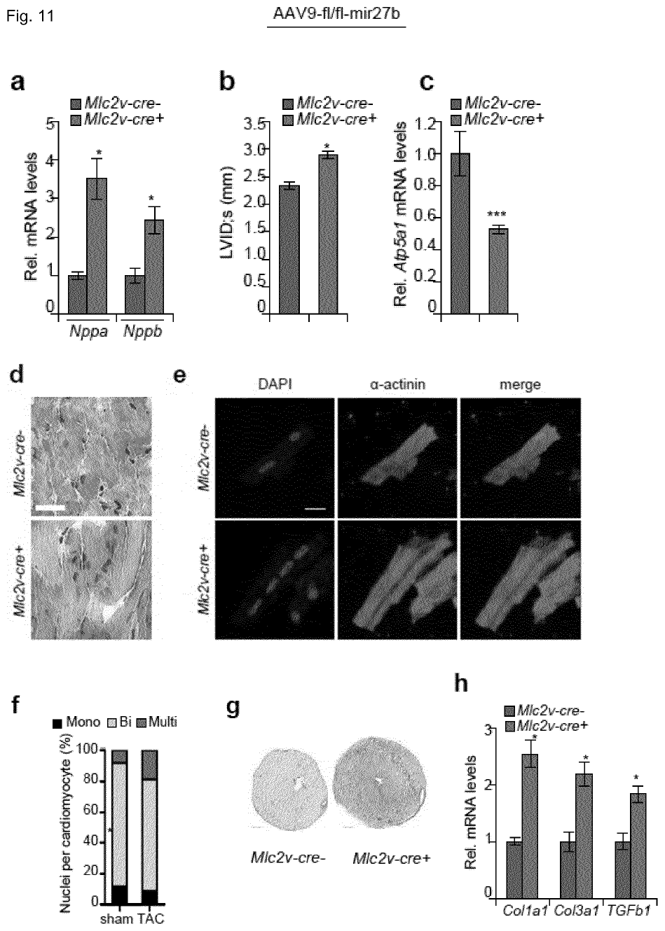

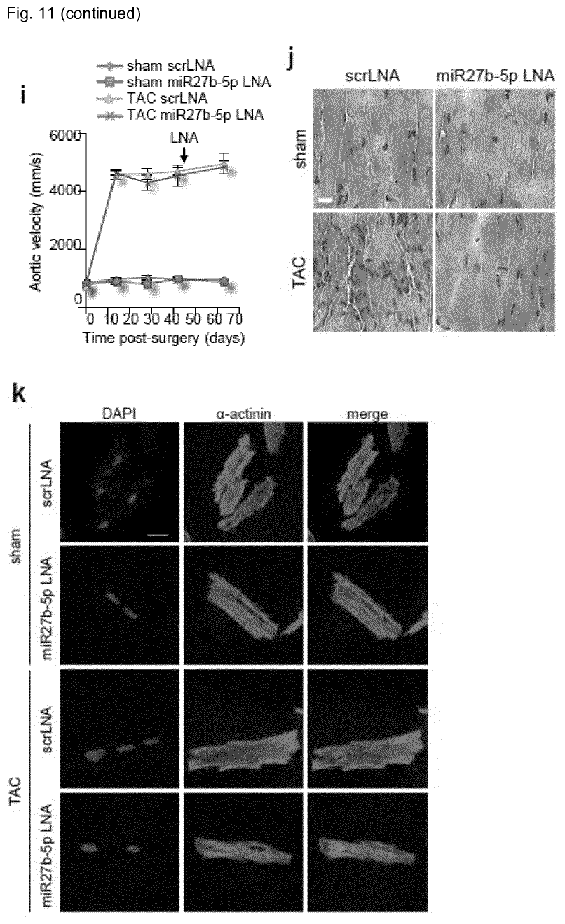

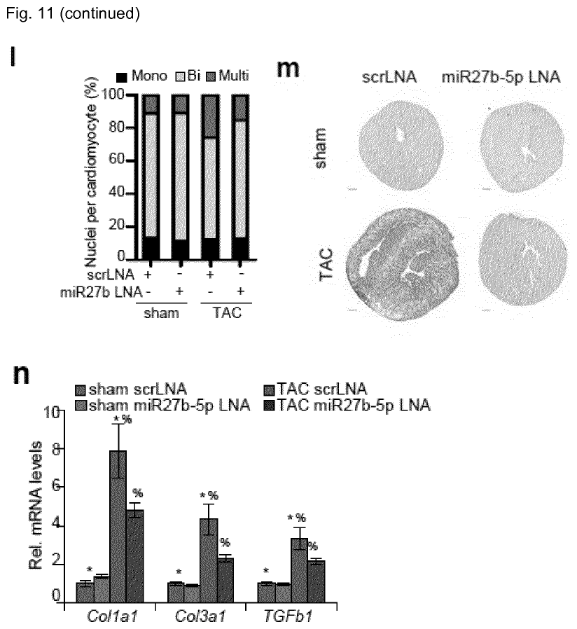

In order to assess if MIR27B expression correlates with human cardiac pathology, we profiled its expression in healthy and diseased human cardiac biopsies. As noted in FIG. 2a, MIR27B expression was elevated in left ventricular biopsies of HCM and AS patients compared to healthy subjects, thus inversely correlating with ATP5A1 expression (FIG. 1a). We examined the causal association between mir27b and Atp5a1 in vivo through delivery of AAV9 carrying pre-mir27b (AAV9-fl/fl-mir27b) to Mlc2v-Cre transgenic mice to achieve ectopic mir27b expression specifically in the ventricular myocardium. As noted ectopic mir27b expression was sufficient to drive cardiac overgrowth, contractile dysfunction and Atp5a1 mRNA and protein repression at 11 weeks after transduction (FIG. 2b-h,). Furthermore, an increase in the fraction of multinucleated cardiomyocytes, as a readout of elevated endoreplication, was observed in left ventricles of Mlc2v-Cre⁺ mice compared to Cre⁻ littermates (FIG. 2i,j and FIG. 11d-f), to a comparable extent as observed upon TAC surgery (FIG. 1i,j). Cardiac fibrosis was similarly elevated (FIG. 11g,h). These observations and our identification of miR27b-5p as the principal regulator of Atp5a1 expression (FIG. 10b,f-p) led us to interrogate miR27b-5p function in mice exhibiting severe heart failure—a disease state commonly associated with ATP synthase repression and ATP depletion. C57BL/6J mice were randomly assigned to two groups, with the groups subjected to either sham or TAC surgery and further subdivided into treatment groups with scrambled LNA (scrLNA) or miR27b-5p LNA (FIG. 2k). In order to mimic late-stage human heart failure, TAC surgery was performed and hypertrophy allowed to progress till severe cardiac dysfunction was detected by echocardiography. At which time LNA treatment was initiated and echocardiography performed to monitor disease progression, as indicated in the respective figure panels (FIG. 2k-p). TAC surgery led to an increase in aortic flow velocity, a control for the degree of overload applied on the myocardium, while sham treated mice displayed normal aortic flow (FIG. 11i). Concomitant to severe cardiac hypertrophy, as demonstrated by the increased ventricular weight:body weight ratio and ventricular dilatation, systolic cardiac dysfunction was evident by a dramatically decreased ejection fraction (EF) (FIG. 2l-p and Table 3) in TAC mice at 42-days post-surgery. Following this observation, mice from the respective groups were subdivided for scrLNA or miR27b-5p LNA therapy. Longitudinal monitoring of disease progression by echocardiography revealed an absence of pathology in sham operated mice. TAC operated mice treated with scrLNA displayed a further decline in cardiac function and heart failure as evidenced by pronounced hypertrophy development, ventricular dilatation and reduced cardiac ejection fraction (FIG. 2l-p and Table 3). In contrast, TAC mice treated with miR27b-5p LNA exhibited improved cardiac function and partial reversion of hypertrophic growth (FIG. 2l-p and Table 3). Analysis of miR27b-3p and miR27b-5p expression confirmed specific inactivation of the miR27b-5p species (FIG. 2q,r), correlating with reduced hypertrophic marker gene expression (FIG. 2s) and increased Atp5a1 mRNA and protein expression compared to scrambled LNA treated TAC operated mice, mimicking expression levels in sham-treated mice (FIG. 2t,u). Strikingly, the stress-induced increase in multinucleation detected by immunofluorescent staining of left ventricular sections from TAC operated mice injected with scrambled LNAs was markedly reduced in TAC operated mice receiving miR27b-5p LNAs (FIG. 2v,w and FIG. 11j-l). Likewise, miR27b-5p LNA therapy significantly reduced cardiac fibrosis and mortality in mice subjected to TAC surgery compared to mice that were injected with scrambled LNAs (FIG. 2x and FIG. 11m,n). Thus, pathologic-stress induced MIR27B-5p expression and ATP5A1 repression is required for maintenance of key aspects of pathologic cardiac growth in humans and mice.

### Mitochondrial ADP Drives Purine Biosynthesis

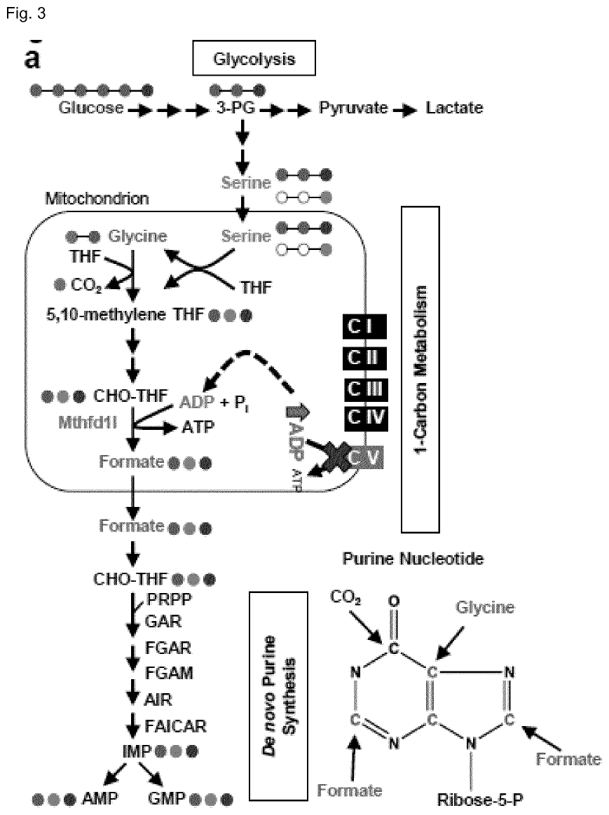

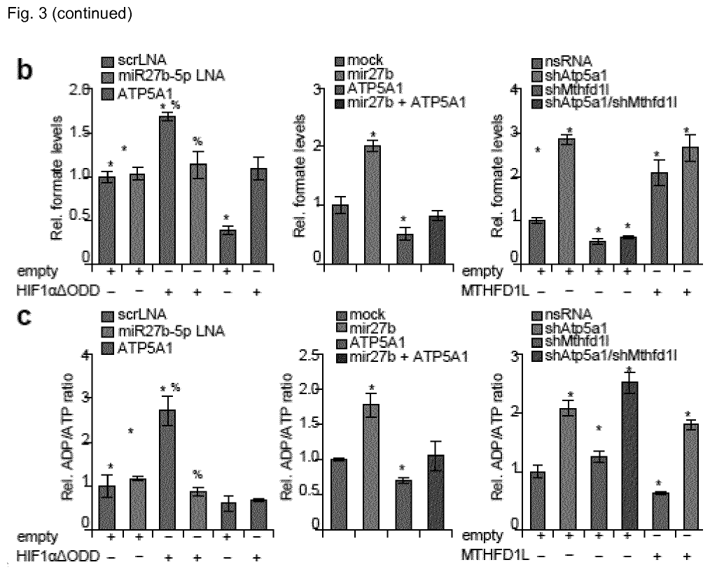

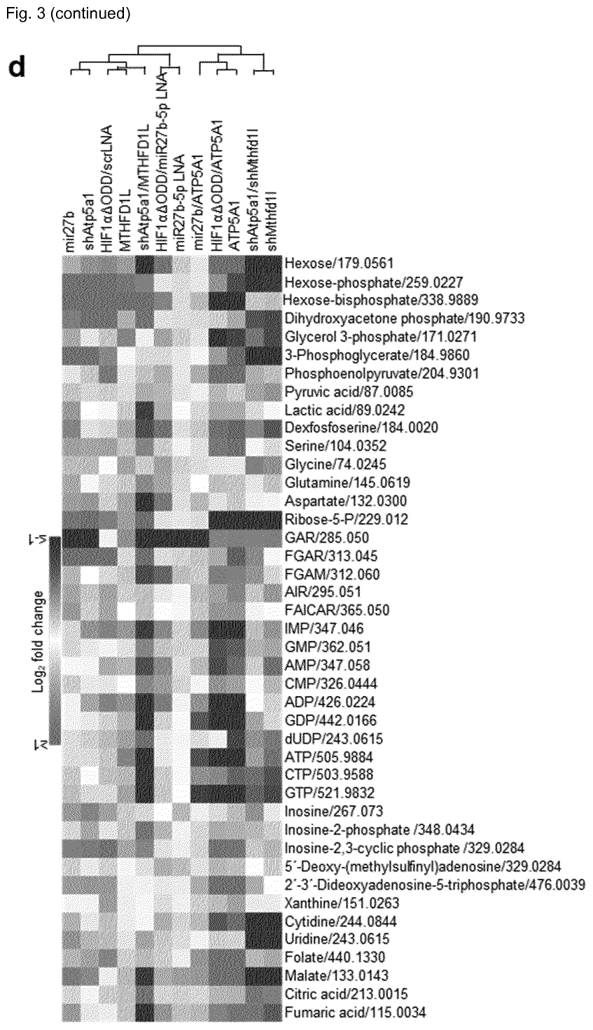

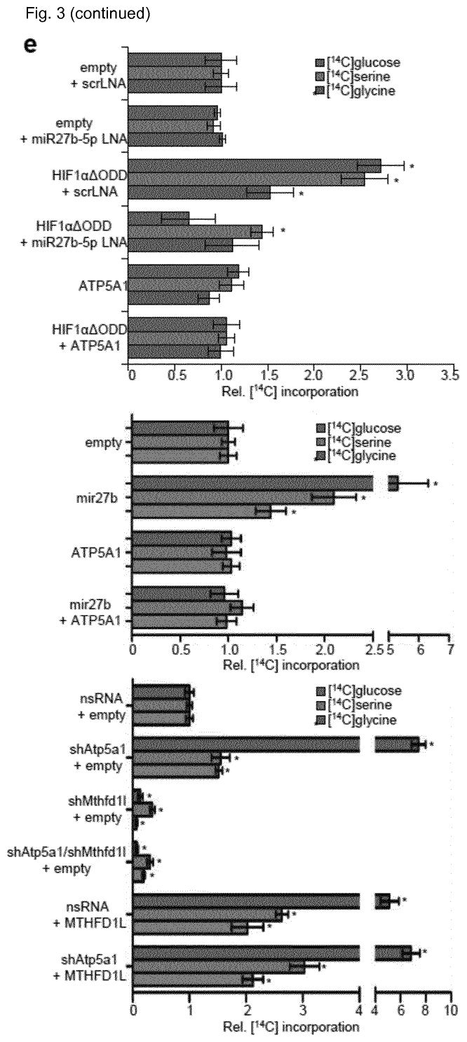

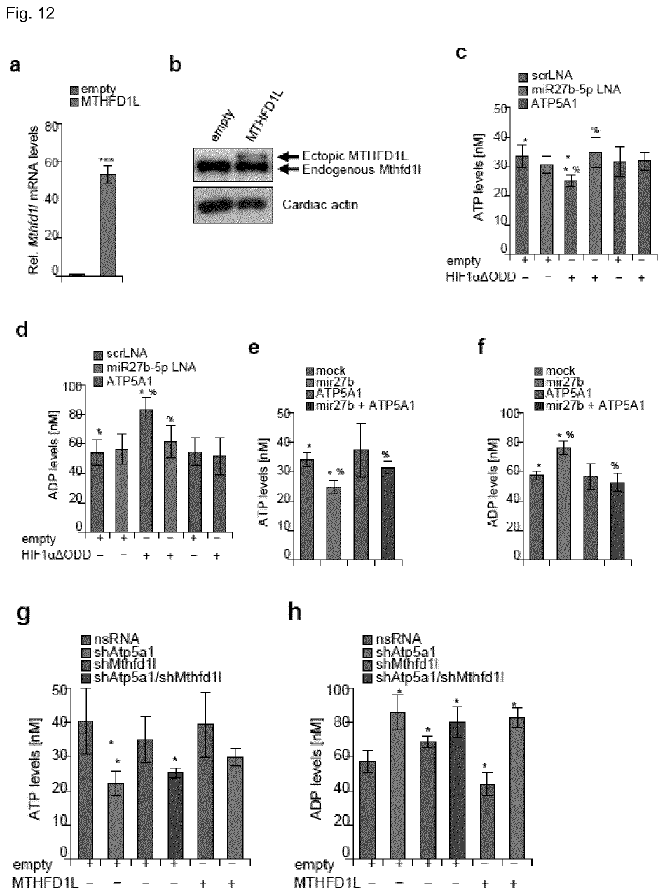

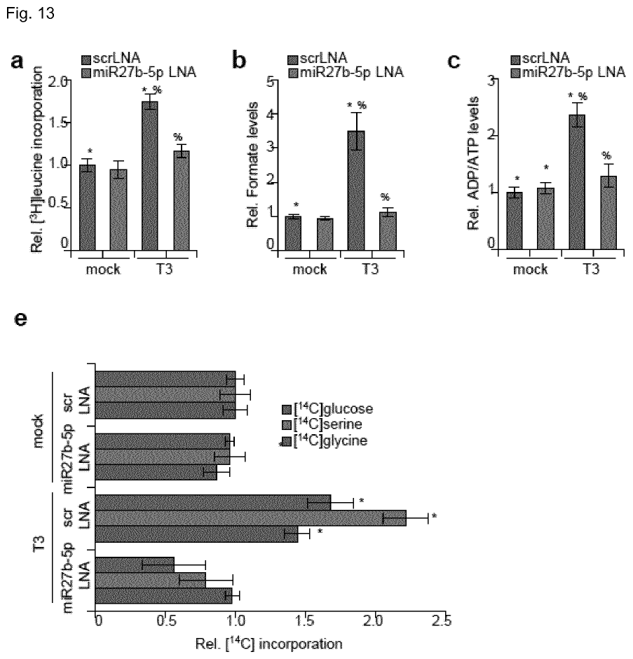

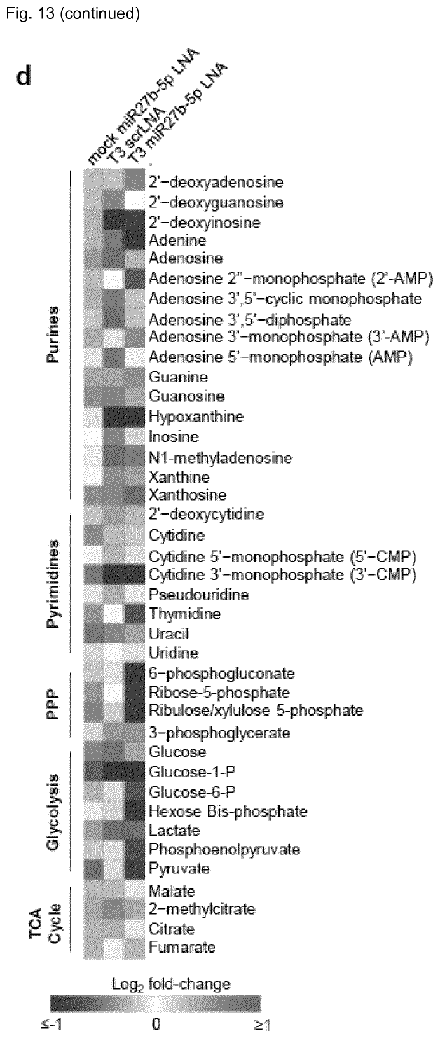

Given that ATP synthase inactivation elevates intra-mitochondrial ADP levels, we explored if ADP in the absence of substrate competition from ATP synthase could be rechanneled to MTHFD1L, an intra-mitochondrial rate-limiting enzyme of the 1-carbon pathway that accounts for 70-99% of formate produced in cells. MTHFD1L utilizes ADP as a rate-limiting cofactor for the hydrolysis of 10-formyltetrahydrofolate (CHO-THF) to formate (FIG. 3a). To test this we measured formate and mitochondrial ATP and ADP levels in genetic Hif1a, miR27b, Atp5a1 and Mthfd1l gain- and loss-of-function settings (FIG. 3b,c, FIG. 12a-h). Increased formate generation and ADP:ATP ratios correlated directly with ectopic Hif1α or miR27b expression, and shRNA-mediated Atp5a1 knockdown (FIG. 3b,c). Next, we performed metabolomics in similar settings as above to investigate the metabolic link between activation of the HIF1α-MIR27B-ATP5A1 axis and elevated levels of multinucelation and cardiomyocyte growth (FIG. 3d). Strikingly ectopic Hif1a, miR27b or shAtp5a1 expression resulted in increased levels of glucose and glycolytic intermediates (Dihydroxyacetonephosphate, 3-Phosphoglycerate) pointing to increased glucose uptake and glycolysis. Moreover, elevated levels of serine and glycine, intermediates of the purine biosynthesis pathway (Ribose-5-phosphate, FGAR) as well as purine and pyrimidine derivatives (Inosine, Xanthine, dUTP, GTP, Uridine) were observed. In contrast, concomitant miR27b-5p inactivation, depletion of Mthfd1l or ectopic ATP5A1 expression partially reversed these effects (FIG. 3d). Moreover, ectopic expression of MTHFD1L alone was not sufficient to induce the de novo purine biosynthesis pathway, supporting the view that mitochondrial ADP serves preferably as a substrate for ATP Synthase and that the induction of mitochondrial formate biosynthesis is highly dependent on repression of ATP5A1 (FIG. 3d). Similar results were observed with T3, resulting in highly miR27b-dependent growth (FIG. 13a), formate production (FIG. 13b) and ADP/ATP increase (FIG. 13c). LC-MS based analysis of the metabolome revealed increased production of purine and pyrimidine derivatives as a function of miR27b expression (FIG. 13d).

Thus, ADP rechanneling connects energetic compromise to stress-induced biosynthesis pathways.

Purines are essential for nucleic acid synthesis, endoreplication and cell growth. Its de novo synthesis can be traced to the glucose that enters the cell and undergoes glycolysis to form 3-phosphoglycerate (3-PG), and further metabolized to generate serine and (indirectly) glycine via the serine biosynthesis pathway, which then serve as key 1-carbon donors through incorporation of its carbon into the purine ring (FIG. 3a). As the carbon atom incorporated into the purine ring essentially traces back to glucose carbons, we followed the flux of carbon into the purine ring of nucleic acids through labeling of glucose, serine and glycine, respectively (FIG. 3e). As shown, increased incorporation of carbon atoms derived from glucose, serine and glycine was observed in the nucleic acid fraction of cells ectopically expressing HIF1αΔODD, miR27b or shAtp5a1 (FIG. 3e), while simultaneous ectopic MTHFD1L expression did not appreciably increase contribution of labeled carbons to the nucleic acid fraction (FIG. 3e), in line with the obtained metabolome profile (FIG. 3d). Similar effects were observed in settings of T3-induced cell growth and miR27b-5p inactivation (FIG. 13e). These data suggest that the entire upstream network impinges on the crosstalk between ATP5A1 and MTHFD1L in regulating pathology-induced de novo nucleic acid synthesis.

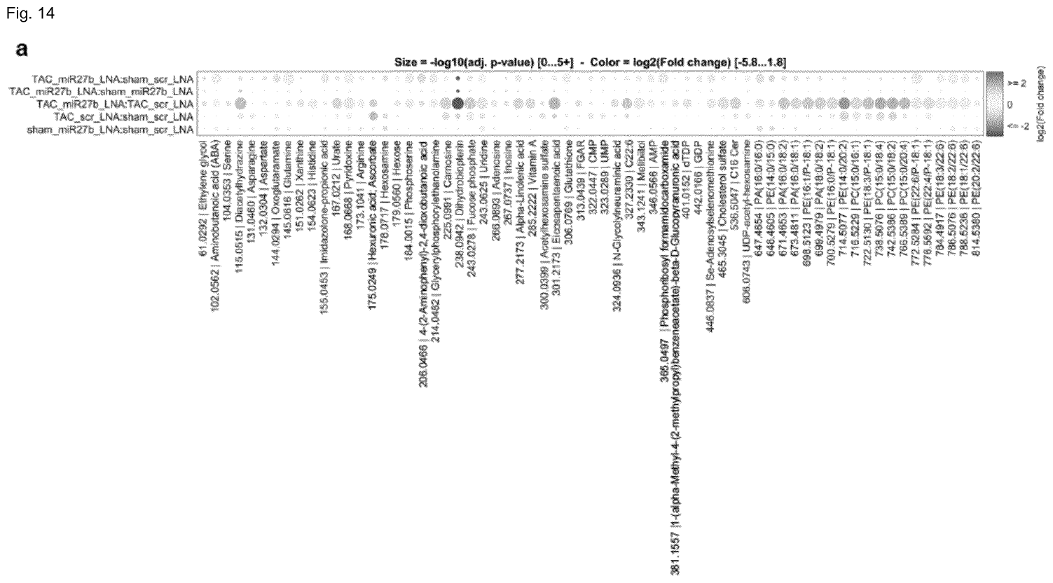

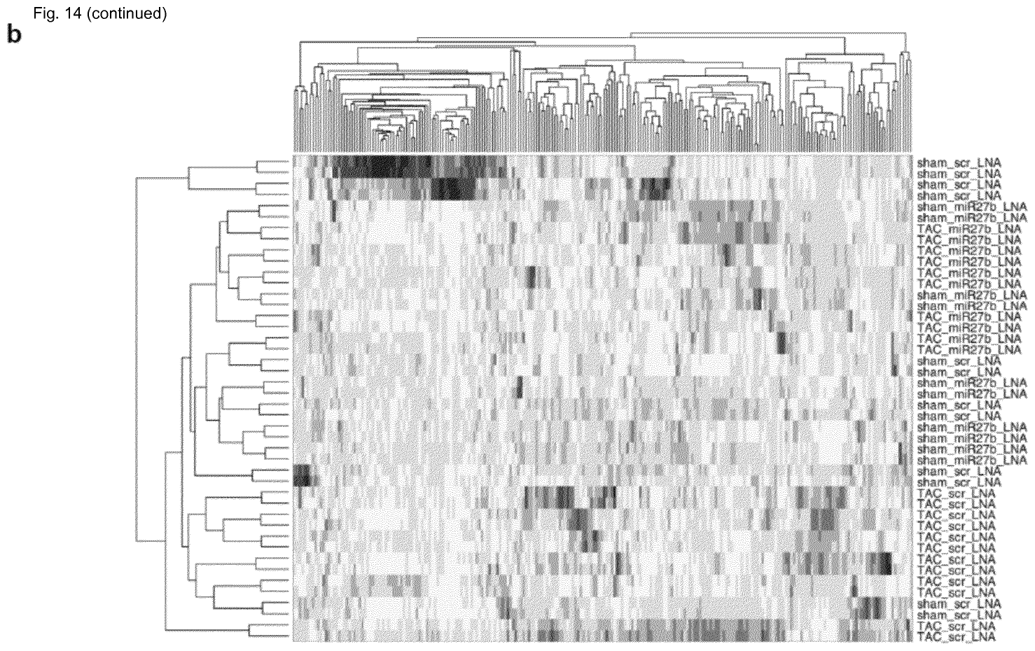

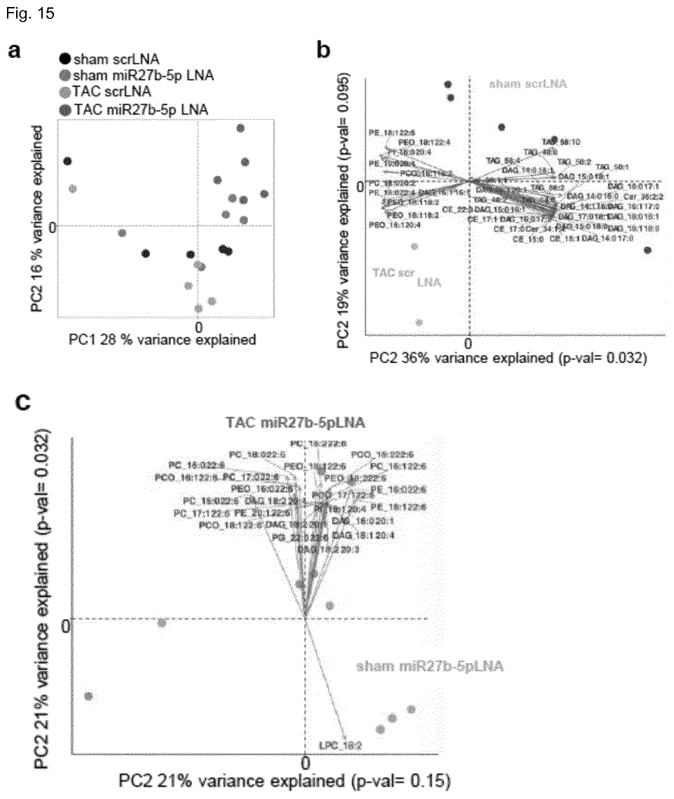

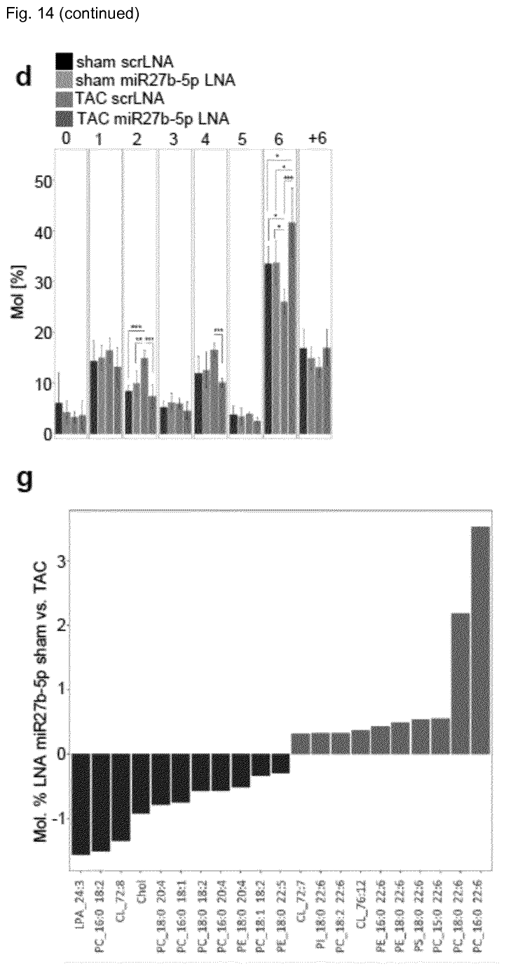

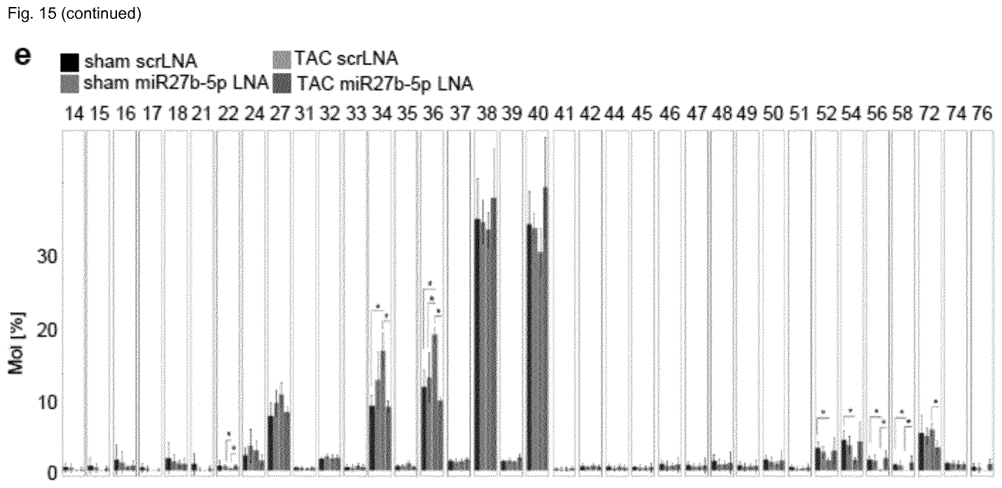

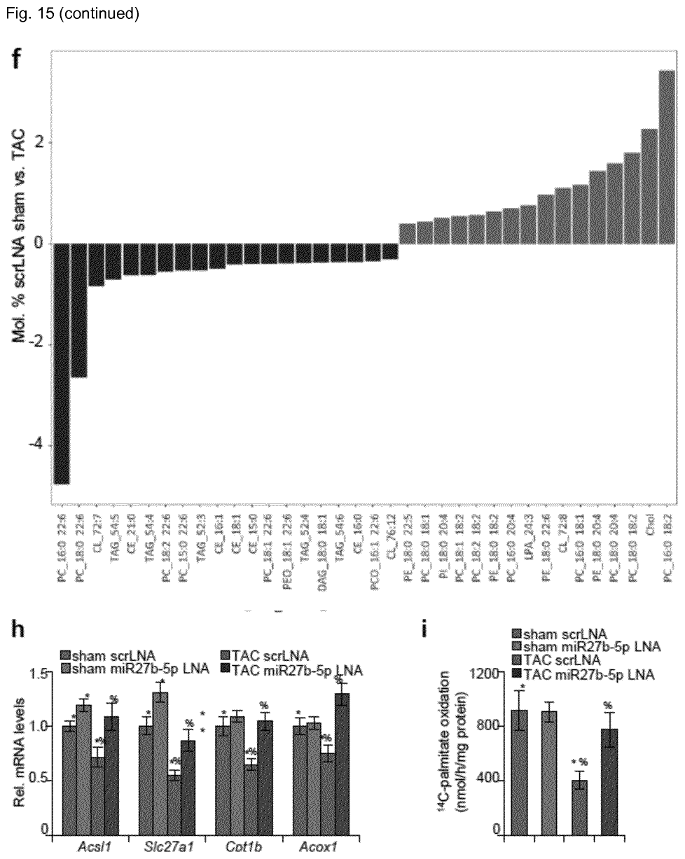

Finally, we determined if similar changes in metabolite distribution could be detected in left ventricular biopsies of mice subjected to either sham or TAC surgery with scrambled LNA (scrLNA) or miR27b-5p LNA treatment (FIG. 2k-p). As noted in FIG. 14a, the metabolite changes observed in cardiac left ventricles closely paralleled changes observed in vitro above, including the elevation of purine precursors such as N-Formylglycinamide ribonucleotide (FGAR) and 5-Formamidoimidazole-4-carboxamide ribotide (FAICAR) in scrLNA treated TAC mice compared to TAC-operated animals treated with miR27b-5p LNA or scrLNA treated mice subjected to sham surgery. Furthermore, the purines xanthine and inosine were significantly lower abundant in miR27b-5p treated TAC mice compared to TAC-operated mice treated with scrLNA (FIG. 14a). Hierarchical clustering revealed that the metabolic signature of left ventricular biopsies from TAC-treated mice injected with miR27b-5p LNAs was altered compared to scrLNA injected TAC-mice, but comparable to that of of sham-operated scrLNA and miR27b-5p LNA treated mice (FIG. 14b). In particular, TAC-operated left ventricular biopsies of miR27b-5p LNA injected mice revealed lower levels of free fatty acids and other lipid species compared to TAC scrLNA controls (FIG. 14a). Thus, we have in addition investigated the lipidome in the above-named in vivo samples (FIG. 15a-g). Principal component analyses (PCA) indicated a unique lipidomic signature in left ventricles of TAC-operated scrLNA vs. miR27b-5p LNA injected mice (FIG. 15a). Lipidomes for sham- or TAC-operated scrLNA treated hearts partially overlap, but significantly separate along the first Principal Component (PC1) whereas lipidomes of sham-or TAC treated hearts with diminished miR27b-5p expression levels significantly separate along the second principal component (PC2) (FIG. 15b,c). A closer look into the lipid composition revealed that left ventricular biopsies of TAC-operated/miR27b-5p LNA injected mice show indeed the highest amount of lipids containing 6 double-bonds (db), while TAC scrLNA samples significantly stand out for 2 db, 4 db and for lipids with 34 and 36 carbon atoms (FIG. 15d,e).

Analyzing the abundance of individual lipid species in TAC-operated mice minus mean mol % abundance of lipid species in corresponding controls revealed an enrichment of arachidonic (20:4) and linoleic (18:2) acids in TAC samples (FIG. 15f). Strikingly, lipids containing docosahexaenoic acid (22:6) accumulate in the left ventricle of TAC-operated mice with repressed miR27b-5p expression (FIG. 15g). In line with this result, free docosahexanoic acid was also increased in the untargeted metabolic analysis (FIG. 14a). Taken together the comprehensive analysis of the cardiac lipidome revealed a correlation between the prevention of left ventricular growth in response to pressure overload and an enrichment of polyunsaturated lipids, especially lipids containing docosahexanoic acid, which has been shown to be cardioprotective. Increased hypertrophy on the other hand is linked with a significant upregulation of lipids containing fatty acids with 34 and 36 carbons, pointing to decreased fatty acid oxidation. In accord with the greater oxidative phenotype of hearts derived from TAC mice treated with miR27b-5p LNA (compared to mice subjected to TAC and treated with scrLNA) (FIG. 14a), lipid catabolism was markedly elevated in miR27b-5p LNA-treated mice, as evidenced by enrichment of long chain fatty acids in TAC-operated hearts and treated with scrLNA compared to miR27b-5p LNA-treated TAC hearts (FIG. 15a-g). Furthermore, an increased expression of lipid catabolism mediators was observed in miR27b-5p LNA-treated TAC hearts (compared to mice subjected to TAC and treated with scrLNA), corresponding to an increased palmitate oxidation capacity in miR27b-5p LNA-treated TAC hearts (FIG. 15h,i). In sum, these analyses revealed a unique lipid signature for miR27b-5p LNA-treated TAC hearts characterized by a significant upregulation of lipids bearing the cardioprotective docosahexanoic acid and an increased fatty acid oxidation capacity.

### ATP Synthase Controls Cardiomyocyte Endoreplication and Pathologic Growth

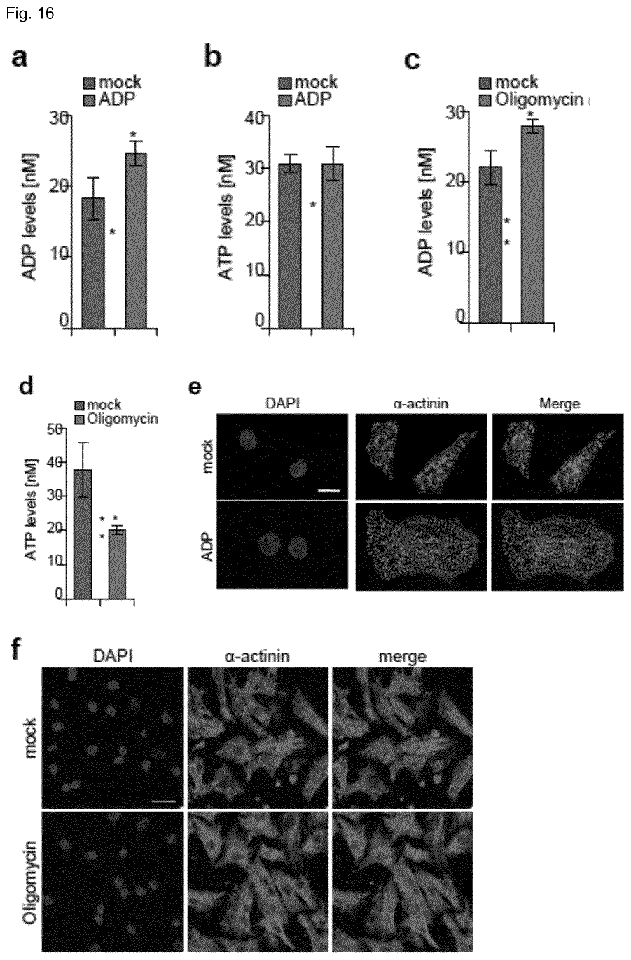

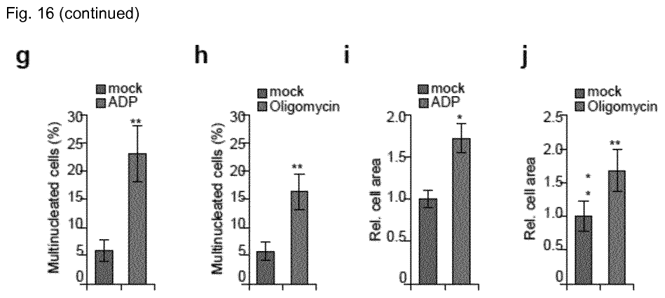

Cardiomyocyte endoreplication precedes pathologic cardiac overgrowth (Ahuja, P., et al., 2007, Physiol Rev, 87, 521-544). To understand if the axis identified indeed contributes to cardiomyocyte multinucleation and growth through ADP, the inventors mimicked miR27b-mediated ATP5A1 repression through treatment of primary cardiomyocytes with oligomycin (an F₀ subunit ATP synthase inhibitor (Shchepina, L. A., et al., 2002, Oncogene, 21, 8149-8157).) or stimulated primary cardiomyocytes with ADP (Hu, J., et al., 2014, Cell Death Dis, 5, 1424). As noted in FIG. 16a-d, ADP levels were indeed elevated in cells treated with oligomycin or ADP. Next, the inventors assessed the sufficiency for oligomycin or ADP to induce endoreplication and multinucleation, as visualized by immunofluorescent staining with DAPI and skeletal α-Actinin to quantify the fraction of multinucleated cells (FIG. 16e-h), and cardiomyocyte hypertrophy by cell size quantification (FIG. 16i,j).

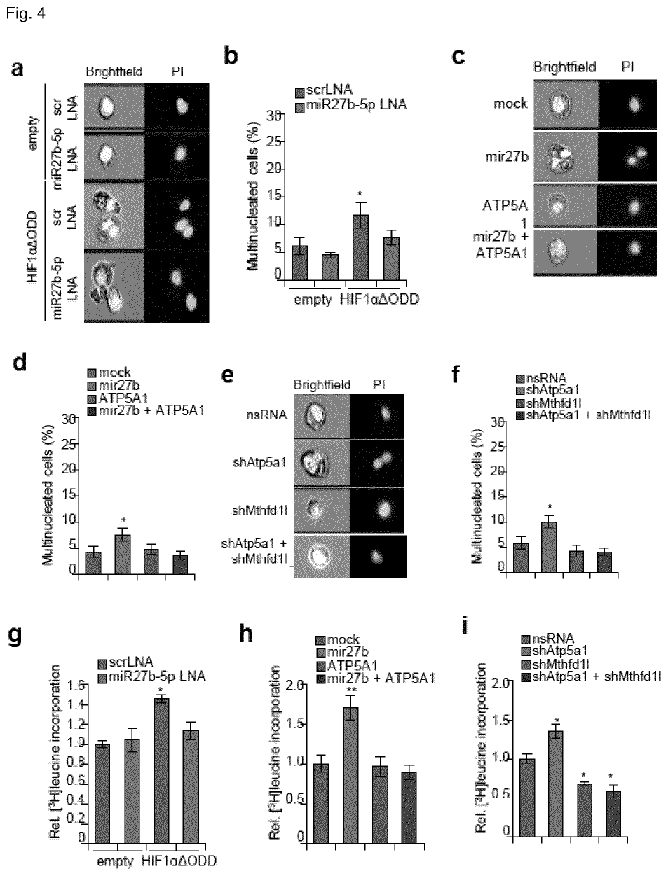

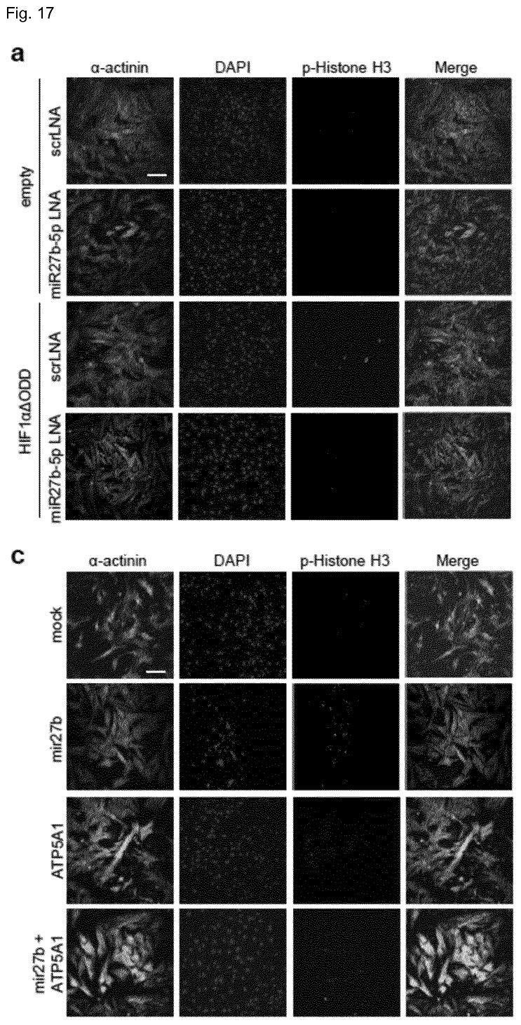

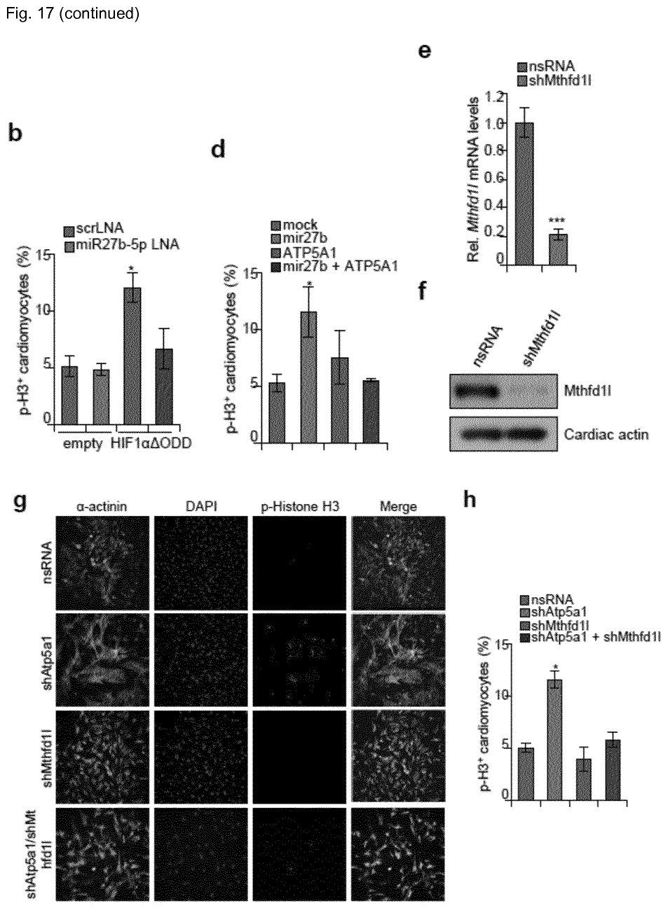

To better define the underlying mechanism, the inventors analyzed cardiomyocyte ploidy and cell size in Hif1a, miR27b, Atp5a1 and Mthfd1l gain- and loss of function settings (FIG. 4a-i and FIG. 17a-h). As shown by imaging coupled flow cytometry and flow cytometry of propidium iodide (PI) stained DNA, ectopic HIF1α, mir27b or shAtp5a1 expression was sufficient to increase the population of multinucleated polyploid cells (FIG. 4a-f), an effect reverted upon simultaneous miR27b-5p LNA-mediated inactivation (FIG. 4a,b), ectopic ATP5A1 expression (FIG. 4c,d) or depletion of Mthfd1l by co-transduction of shMthfd1l expressing lentiviruses (4e,f). In order to directly visualize endoreplicated multinucleated cells, cardiomyocytes were stained for phosphorylated histone 3 at serine 10 (p-Histone H3) and imaged by confocal microscopy (FIG. 17a-h). Phosphorylated histone H3 marks condensed chromosomes that are characteristic of karyokinetic cells. Consistent with the flow cytometry data, ectopic HIF1αΔODD expression led to increased phosphorylated histone H3 stained nuclei which was rescued upon parallel miR27b-5p inhibition with LNAs (FIG. 17a,b). Furthermore, mir27b overexpression or Atp5a1 inhibition similarly led to increased numbers of karyokinetic cells compared to corresponding controls (FIG. 17c,d). shRNA-mediated Mthfd1l inhibition led to efficient depletion of its mRNA and protein (FIG. 17e,f) and resulted in comparable phospho-Histone H3 staining as control cells (FIG. 17g,h). Remarkably, simultaneous expression of ectopic ATP5A1 and mir27b, or co-transduction of NRC with shMthfd1l and shAtp5a1 lentiviruses likewise reduced the extensive pattern of phospho-Histone H3 staining as observed upon ectopic expression of mir27b or shAtp5a1 alone (FIG. 17c-h). To verify that the increase in de novo purine biosynthesis and multinucleation links directly to cell growth, the inventors performed leucine-incorporation assays under identical treatment conditions. As shown in FIG. 4g-i ectopic HIF1αΔODD, mir27b expression, or Atp5a1 knockdown led to increased leucine-incorporation (indicative of increased protein synthesis and cell growth), which was negated upon simultaneous miR27b-5p inactivation (FIG. 4g), or ectopic expression of ATP5A1 (FIG. 4h). In line with the critical requirement for MTHFD1L in purine biosynthesis, the fraction of multinucleated cells was reduced upon simultaneous depletion of MTHFD1L and ATP5A1 (FIG. 4e,f) as was pathologic cell growth (FIG. 4i). Thus, our data supports the view that re-programming of the metabolic environment is sufficient to drive cardiomyocyte endoreplication, resulting in multinucleation and cell growth.

### MTHFD1L is a Key Modulator of Pathologic Cardiac Growth

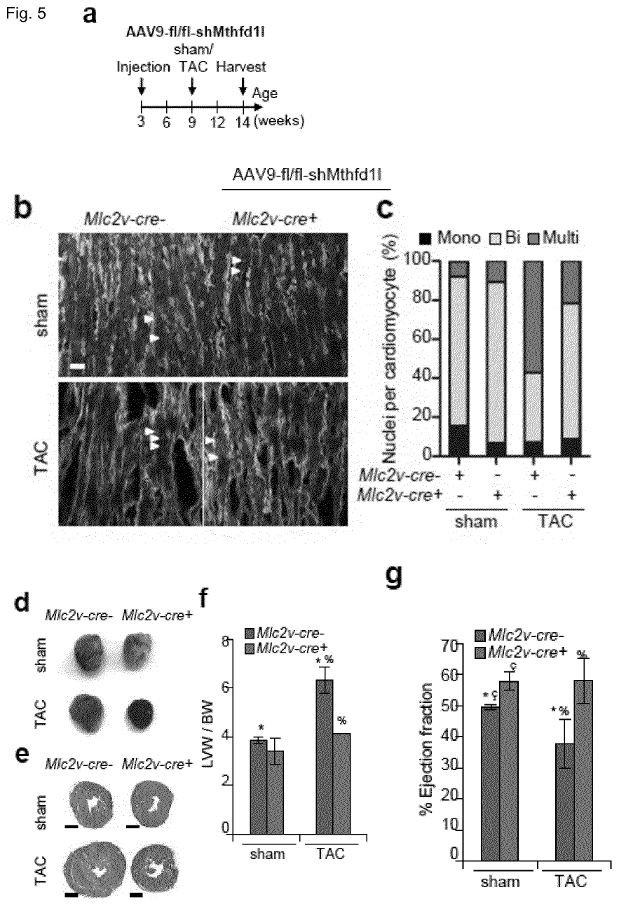

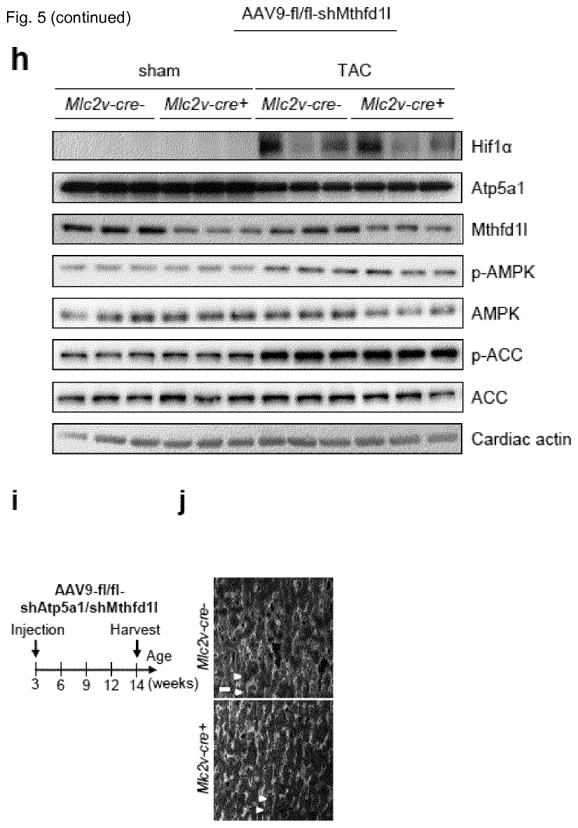

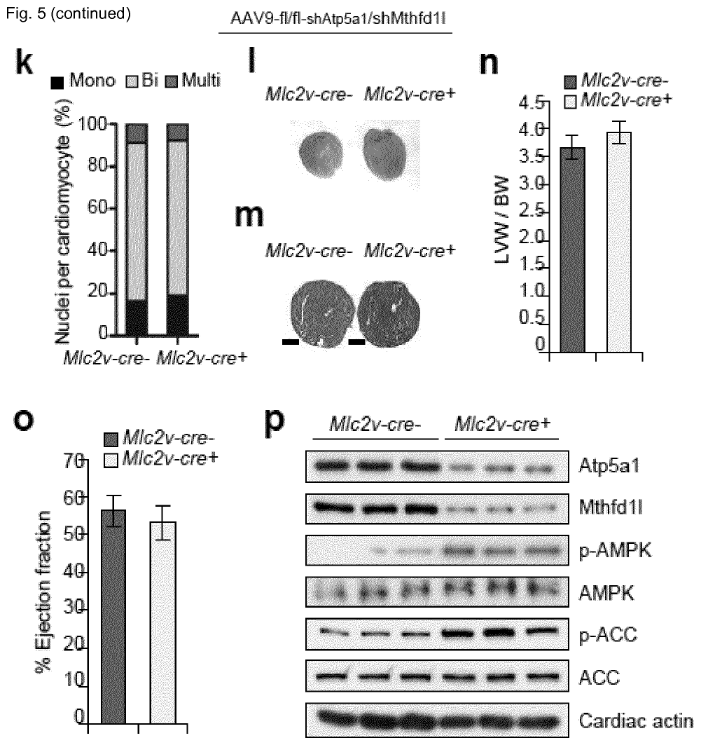

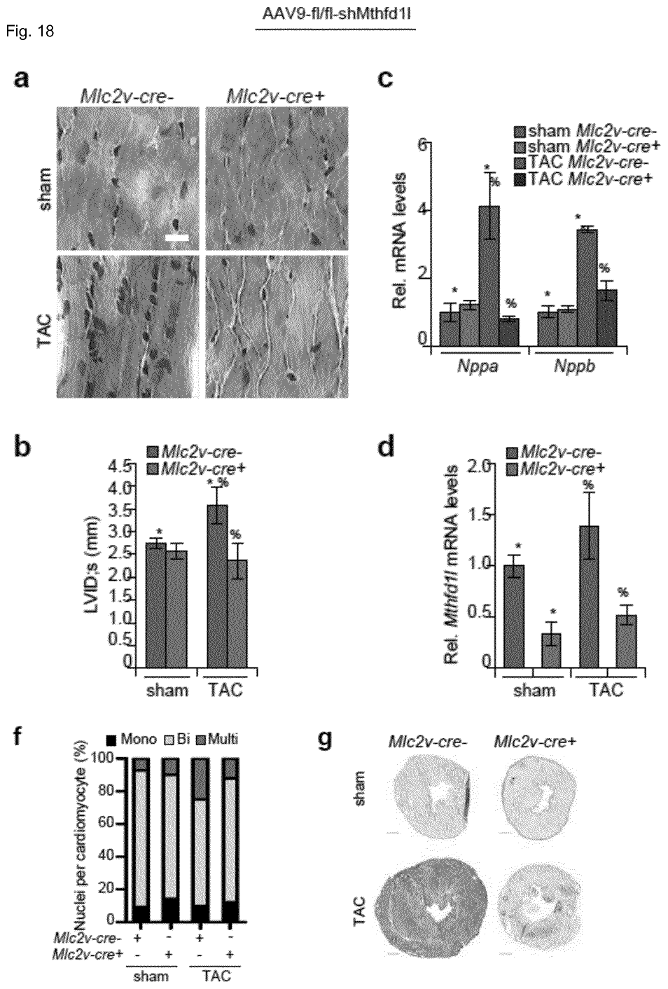

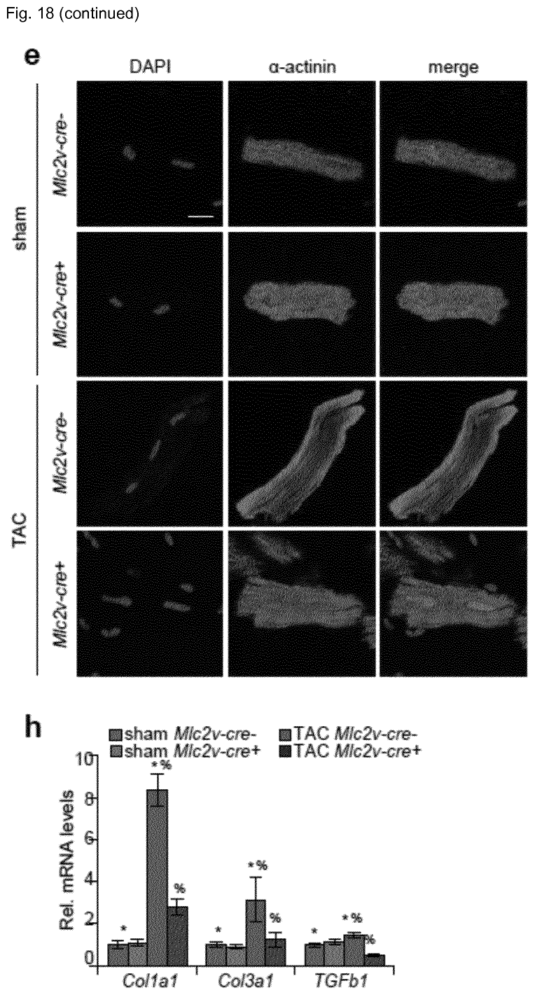

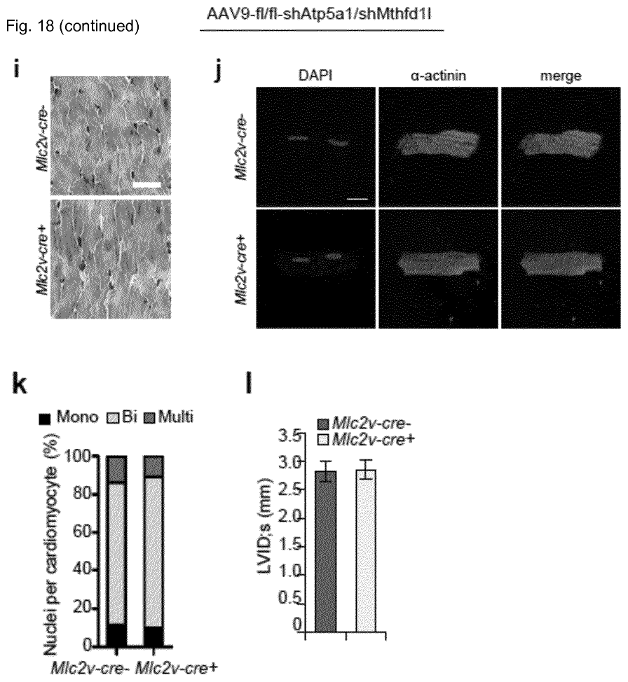

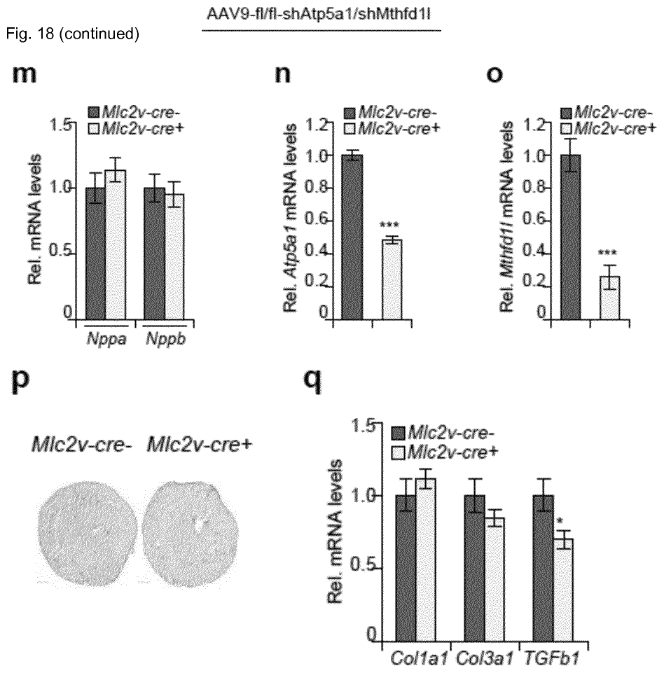

To confirm that MTHFD1L impacts cardiac pathologic hypertrophy, the inventors injected Mlc2v-cre⁻ and cre⁺ mice subjected to sham or TAC surgeries with AAV9 harboring short-hairpin RNAs targeting Mthfd1l (AAV9-fl/fl-shMthfd1) (FIG. 5a). Consistent with the previous TAC data (FIG. 1i,j), multinucleation was significantly increased in Mlc2v⁻ cre mice, as quantified from immunofluorescent staining (FIG. 5b,c and FIG. 18a). Concomitant with stress-induced endoreplication, Mlc2v⁻ cre mice developed pathologic cardiac hypertrophy and systolic dysfunction after TAC surgery, evident by increased left ventricular weight:body weight ratio, increased left ventricular internal diameter in diastole (LVId) and eleveated hypertrophic marker genes and reduced ejection fraction compared to sham-operated Mlc2v⁻ cre mice. (FIG. 5d-g, FIG. 18b-d and Table 4). Strikingly, the percentage of multinucleated cells was significantly lower in TAC operated Mlc2v-cre⁺ mice compared to similarly treated Mlc2v-cre littermates (FIG. 5b,c and FIG. 18a,e,f). In accord, cardiac hypertrophy, fibrosis and systolic dysfunction was significantly reduced (FIG. 5d-g, FIG. 18b,g,h and Table 4). qRT-PCR analysis and immunoblotting for Mthfd1l expression revealed sufficient repression of Mthf1l on both the RNA and protein level by AAV9-fl/fl-shMthfd1l transduction (FIG. 5h and FIG. 18d) coincident with resistance to TAC-induced cardiac overgrowth and contractile dysfunction.

In line with the accumulation of HIF1α in TAC treated Mlc2v-cre⁻ and Mlc2v-cre*hearts, Atp5a1 expression was repressed resulting in AMPK phosphorylation at Thr172 and hyperphosphorylation of ACC (FIG. 5h). In line with the fact that Mthfd1l repression was sufficient to prevent increased multinucleation in response to cardiometabolic endoreplication driven by TAC mediated pressure-overload, multinucleation, overgrowth, fibrosis and dysfunction induced by Atp5a1 downregulation (FIG. 1k-s and FIG. 8a-i) was similarly prevented by the parallel inactivation of Mthfd1l in vivo (FIG. 5i-o, FIG. 18i-q and Table 5). Mlc2v-cre⁺ mice co-injected with AAV9-fl/fl-shAtp5a1 and AAV9-fl/fl-shMthfd1l showed no increase in multinucleated cells (FIG. 5j,k and FIG. 18i-k), normal ventricular size (FIG. 5l,m and FIG. 18l), left ventricular weight:body weight ratio (FIG. 5n), hypertrophic marker gene expression (FIG. 18m) and systolic left ventricular function (FIG. 5o and Table 5), despite efficient inhibition of Atp5a1 (FIG. 5p and FIG. 18n) when Mthfd1l was inhibited simultaneously (FIG. 18o) indicating that Mthfd1l functions downstream of Atp5a1. Levels of cardiac fibrosis were similarly comparable between groups (FIG. 18p,q). Thus, ATP5A1 repression and the resulting rechanneling of ADP to MTHFD1L and the consequent increase in purine biosynthesis, drives cardiometabolic endoreplication and multinucleation to enforce pathologic cardiac growth.

### ATP5A1 Integrates Metabolic and Growth Signaling to Drive Pathologic Growth

AMPK activation has been shown to drive inhibition of the tumor suppressor Retinoblastoma protein (Rb) via phosphorylation at Ser804. Rb restricts DNA replication and cell cycle progression from G1 to S through its sequestration and inhibition of E2F transcription factors, which are composed of dimers E2F protein and a dimerization partner (DP) protein (E2F-DP). Rb phosphorylation results in the release and de-repression of E2F transcription factors from the E2F-DP complex, culminating in E2F target gene induction and cell cycle re-entry. Given that the HIF1α-miR27b-ATP5A1-MTHFD1L axis creates a metabolic context permissive for cell cycle re-entry and endomitosis in post-mitotic adult cardiomyocytes, the inventors interrogated possible cooperation of metabolic and growth factor signaling in cardiac pathologic hypertrophy.

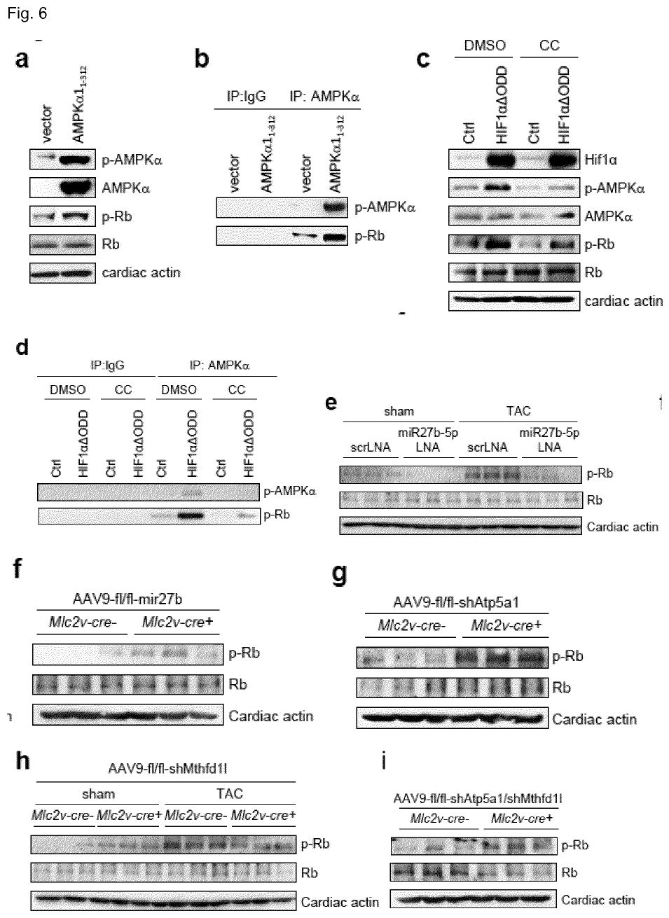

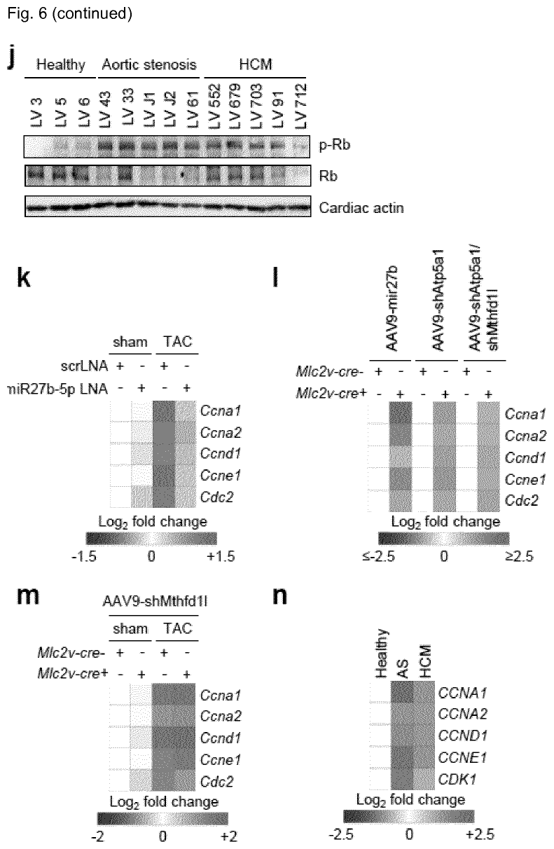

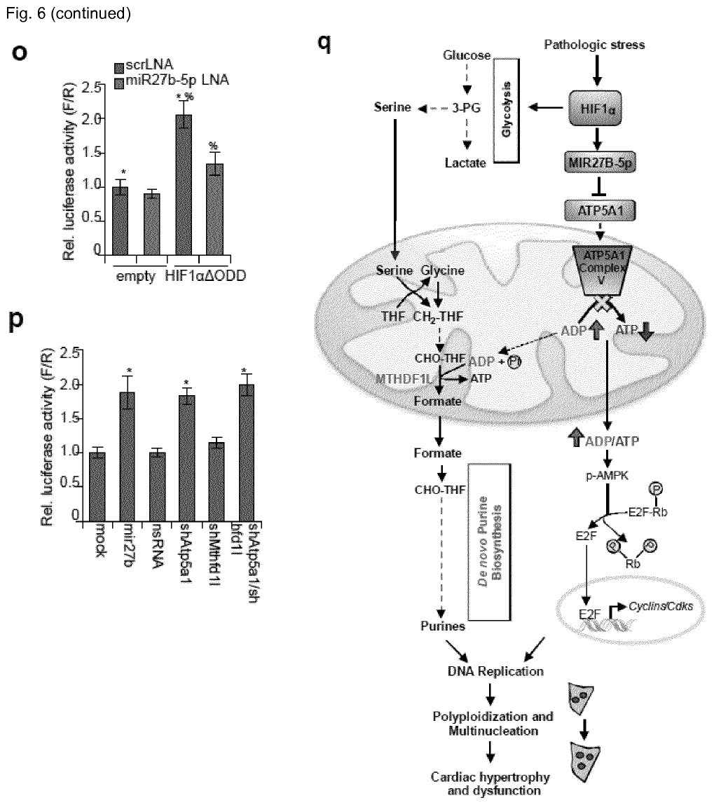

First the inventors confirmed direct interaction of AMPK with Rb in our setting through co-immunoprecipitation of AMPK and Rb. To that end, the AMPKα subunit was ectopically expressed in cardiomyocytes and subsequently immunoprecipitated. Pull-down products revealed co-precipitation of AMPKα with phosphorylated-Rb (FIG. 6a,b). The inventors confirmed this interaction through pull-down of endogenous AMPK in cells expressing ectopic HIF1αΔODD. As noted, AMPK immunoprecipitated with phosphorylated Rb in cardiomyocytes ectopically expressing HIF1αΔODD, but not upon simultaneous inhibition of AMPK with compound C (CC), a potent AMPK inhibitor (FIG. 6c,d).

Having confirmed the direct interaction between AMPK and phospho-Rb, the inventors next assessed if Rb was hyperphosphorylated in our human biopsies and various mouse models (FIG. 6e-j). Indeed, hyperphosphorylation of Rb was detected in lysates of left ventricular biopsies of TAC operated mice injected with scrLNA (FIG. 6e), ectopic mir27b expression (FIG. 6f) and depletion of Atp5a1 (FIG. 6g) in line with the phosphorylation of AMPK at Thr172 under these conditions as demonstrated before (FIG. 1l and FIG. 2h,u). Left ventricles of mice injected with LNAs targeting miR27b-5p (FIG. 6e) or simultaneously transduced with AAV9 bearing shRNAs against Mthfd1l alone or Mthfd1l and Atp5a1 were protected from pRb induction after TAC surgery (FIG. 6h,i). In accord with experiments in mice and the observed Thr172 phosphorylation of AMPK (FIG. 1c) immunoblotting of lysates from ventricular biopsies of AS and HCM patients revealed likewise increased phospho-Rb expression (FIG. 6j). Taken together, pRb is a downstream target of phosphorylated AMPK in the presence of the cardiometabolic endoreplication driving HIF1α-MIR27B-ATP5A1 axis.

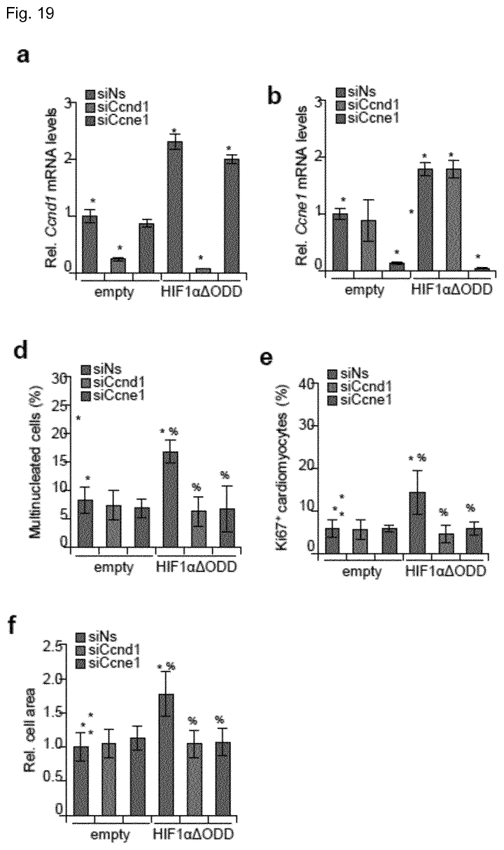

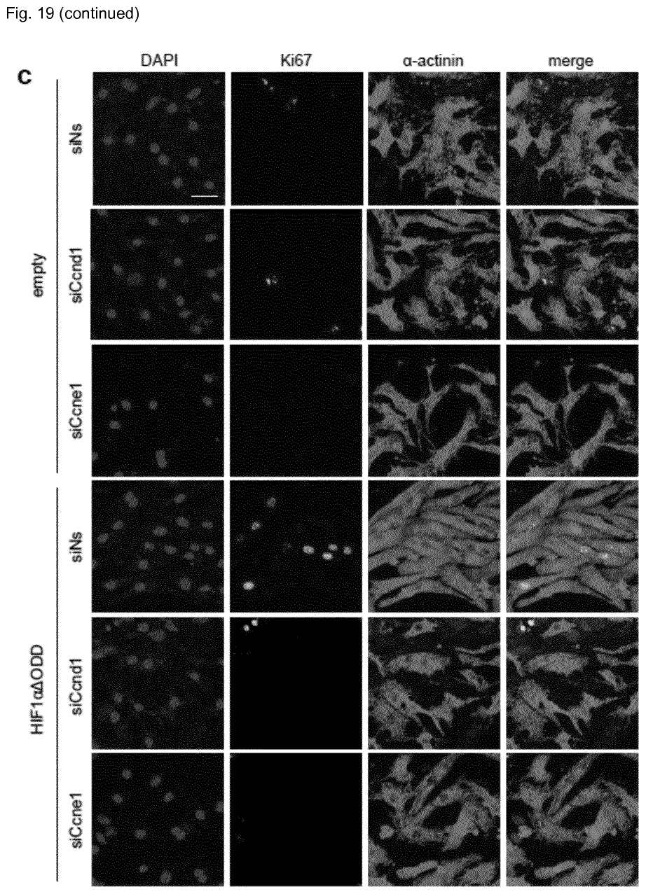

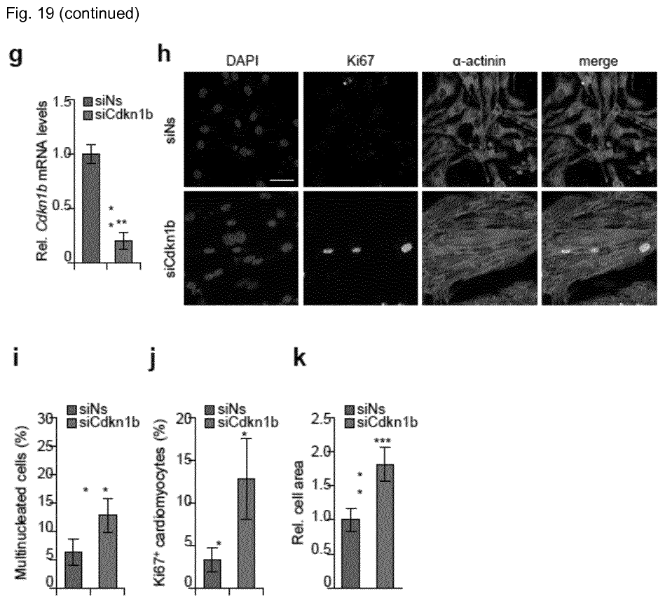

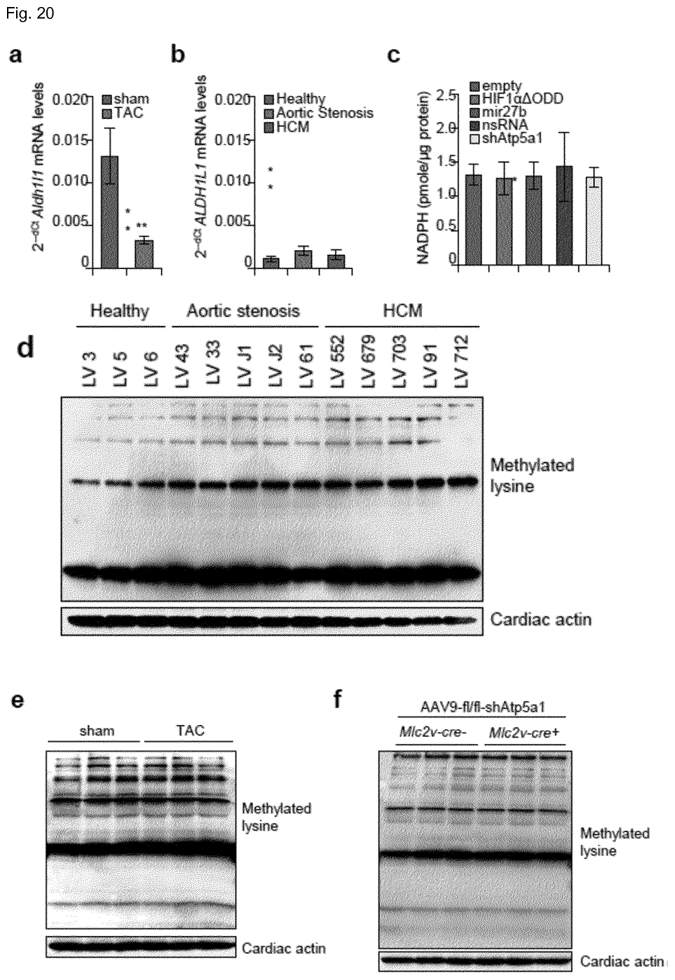

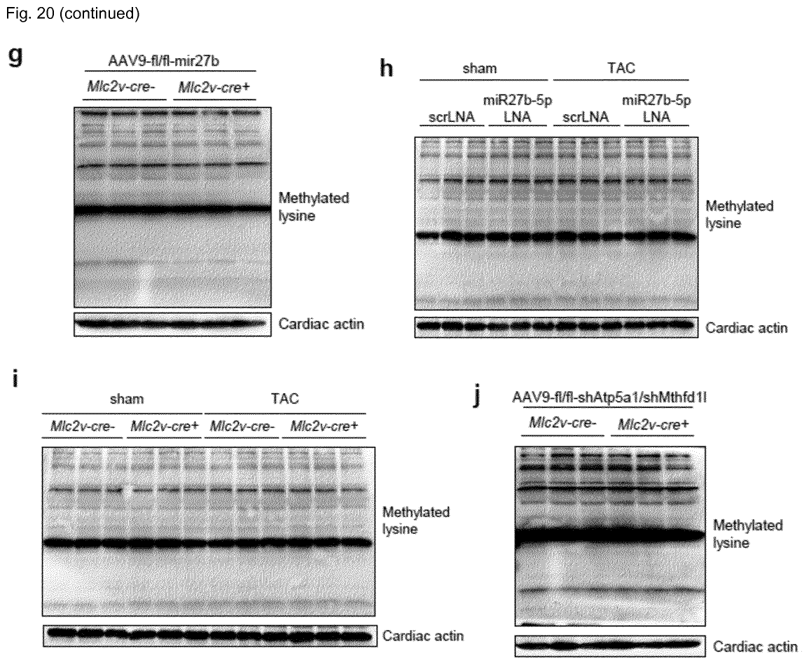

To investigate whether the predicted release of E2F1 upon Rb hyperphosphorylation leads to increased transcription activity of E2F1, the inventors performed qRT-PCRs to detect changes in RNA levels of E2F1-target genes (Cyclin A1, A2, D1, E1 (Ccna1, Ccna2, Ccnd1, Ccne1) and Cyclin-dependent kinase 1 (Cdk1)) on left ventricular samples of mice treated with the above mentioned conditions and on human heart biopsies (FIG. 6k-n). These gene expression analysis could confirm the activation of the E2F pathway in vivo, downstream of phospho-AMPK mediated Rb inhibition in mice and AS and HCM patients (FIG. 6k-n). E2F luciferase reporter assays performed in NRC further confirmed E2F transcriptional activity under ectopic expression of HIF1αΔODD, mir27b or Atp5a1 depletion (FIG. 6o,p). Finally, to functionally assess the impact of cell cycle regulators on cardiomyocyte endomitosis and multinucleation the inventors inactivated Ccnd1 (cyclin D1) and Ccne1 (cyclin E1), two key downstream components of Rb activation in cardiomyocytes expressing ectopic HIF1αΔODD. As evidenced, inactivation of Ccnd1 and Ccne1 inhibited HIF1α-mediated endomitosis, multinucleation and hypertrophy (FIG. 19a-f). In contrast, inactivation of the cell cycle inhibitor Cdkn1b (p27/Kip1) was sufficient to induce cardiomyocyte endomitosis, multinucleation and pathologic growth (FIG. 19g-k). Taken together, the inventors have demonstrated that cardiac endoreplication and multinucleation requires simultaneous activation of de novo purine biosynthesis (as facilitated by the identified HIF1α-MIR27B-ATP5A1-MTHFD1L axis) as well as activation of cell cycle by E2F1. This is reflected by the fact that depletion of Mthfd1l in combination with Atp5a1 inhibition was sufficient to induce AMPK activation and E2F transcription (FIG. 6h,i,l,m) but failed to induce cardiometabolic endoreplication (FIG. 5b,c,j,k).

### Materials and Methods

### Animal Breeding and Maintenance

Hif1α fl/fl mice were obtained from Randall S. Johnson (University of California, San Diego, USA) and Vhl fl/fl mice were kindly provided by Rudolf Jaenisch (Massachusetts Institute of Technology, USA). The myosin light-chain (Mlc)2v-Cre (Chen, J., et al., 1998, Development 125, 1943-1949) line was from Ju Chen (University of California, San Diego, USA). The respective ventricular-specific mouse lines described in this manuscript were generated by crossing loxP-flanked Hif1α (Hif1α fl/fl) (Ryan, H. E., et al., 2000, Cancer Res, 60, 4010-4015) or Vhl (Vhl fl/fl) (Haase, V. H., et al., 2001, Proc Natl Acad Sci USA, 98, 1583-1588) mice to myosin light-chain Mlc2v-Cre transgenic mice. The data presented represent studies with male mice aged 3-20 weeks of the C57BL/6J background. By 3-20 weeks the inventors were referring to the whole duration of the animal experiments. The AAV9 viruses were administered to 3-week old mice. As the AAV9 vectors have a lag phase of approximately 6-10 weeks until it reaches maximal expression, surgeries were performed at 9-11 weeks and hearts harvested at least 11 weeks after AAV9 administration as shown in the experimental outlines in FIG. 1k, FIG. 2b,k and FIG. 5a,i. In TAC experiments, mice were 10-12 weeks old at the beginning of the experiment. Only mice of a similar age (+/−1 week) were used in the corresponding experiments. In experiments with the Mlc2v⁻ cre mice, littermates and mice of a similar age (+/−1 week) were used. After baseline echocardiography mice were randomly assigned to groups, AAV injections, LNA delivery and echocardiography was performed blinded. In experiments including TAC surgery experiments were kept blinded over the baseline echocardiography until operation. All mice were maintained at the MRC Clinical Sciences Centre (Imperial College London), Institute of Molecular Health Sciences (ETH Zurich) and/or the Cardiovascular Assessment Facility (CAF), Department of Medicine, Department of Medicine, University of Lausanne in a specific pathogen-free facility. Maintenance and animal experimentation were in accordance with the Swiss Federal Veterinary Office (BVET) guidelines.

### Human and Mouse Ventricular Biopsies

Human heart biopsies and clinical data were generously provided by Samuel Sossalla (Georg-August-University Goettingen and DZHK, Goettingen, Germany) and Sebastian Stehr (University Hospital Leipzig, Germany). Human HCM and aortic stenosis biopsies were conducted in compliance with the local ethics committee, and written informed consent was received from all subjects prior to inclusion. Myocardial samples were obtained from patients with severe aortic stenosis undergoing aortic valve replacement and a Morrow resection from the hypertrophied left ventricular septum. Only patients without significant aortic valvular regurgitation and with preserved contractile function were included. Human left ventricular biopsies of HCM patients were obtained from left ventricular papillary muscle of explanted hearts. The myocardial samples were acquired directly in the operating room during the surgery and immediately washed in precooled cardioplegic solution (110 mM NaCl, 16 mM KCl, 16 mM MgCl₂, 16 mM NaHCO₃, 1.2 mM CaCl₂, 11 mM glucose) followed by rapid snap-freezing in liquid nitrogen. Healthy heart samples were obtained from left ventricles of donor hearts. Sample weight was approximately 20-150 mg. Clinical data pertaining to these subjects are shown in a previous publication 16

### Transaortic Banding

9-14 week old mice were subjected to transaortic banding (TAC) through constriction of the descending aorta as described (Kassiri, Z., et al., 2005, Circ Res, 97, 380-390). The mice were monitored up to 9 weeks after surgery and their heart dimensions and functions were determined by echocardiography. In vivo miRCURY LNA Power Inhibitors were injected intraperitoneally (i.p.) into C57BL/6J mice at a dose of 10 mg/kg for 4 consecutive days at 49 days post surgery as described in FIG. 2k. The following In vivo miRCURY LNA Power Inhibitors were purchased from Exiqon: i-mmu-miR-27b-5p (199900). i-Cel-control_inh (199900) was used as scrambled control LNA.

### In Vivo Transthoracic Ultrasound Imaging

Transthoracic echocardiography was performed using the MS400 (18-38 MHz) probe from Vevo 2100 color doppler ultrasound machine (VisualSonics). Mice were lightly anesthetized with 1-1.5% isoflurane, maintaining heart rate at 400-550 beats per minute. The mice were placed in decubitus dorsal on a heated 37° C. platform to maintain body temperature. A topical depilatory agent is used to remove the hair and ultrasound gel is used as a coupling medium between the transducer and the skin. Hearts were imaged in the 2D mode in the parasternal long-axis view. From this view, an M-mode cursor was positioned perpendicular to the inter-ventricular septum and the posterior wall of the left ventricle at the level of the papillary muscles. Diastolic and systolic interventricular septum diameter (IVS;d and IVS;s), diastolic and systolic left ventricular posterior wall diameter (LVPW;d and LVPW;s), and left ventricular internal end-diastolic and end-systolic chamber (LVID;d and LVID;s) dimensions were measured. The measurements were taken in three separate M-mode images and averaged. Left ventricular fractional shortening (% FS) and ejection fraction (% EF) was also calculated. Fractional shortening was assessed from M-mode based on the percentage changes of left ventricular end-diastolic and end-systolic diameters. % EF is derived from the formula of (LV vol;d−LV vol;s)/LV vol;d×100. At the end of the duration of the experiment, the animals were sacrificed and the heart weight-to-body weight ratio was measured.

### Isolation and Maintenance of Primary Neonatal Rat Cardiomyocytes

Isolation of primary neonatal rat cardiomyocytes (NRC) was performed using the neonatal heart dissociation kit (130-098-373, Miltenyi Biotec) as recommended by the manufacturer. Isolated cells were pre-plated with plating medium (65% DMEM, 16% M199, 10% fetal calf serum (FCS), 5% horse serum (HS), 2% glutamine and 1% penicillin/streptomycin (P/S)) for 1.5 h to deplete of the fibroblasts. NRC were plated on Type-I Collagen-coated (Advanced Biomatrix) 3 cm dishes (Nunc) in plating medium. The plating medium was changed to maintenance medium (88% DMEM, 9% M199, 1% HS, 2% glutamine and 1% P/S) 24 h after isolation of NRCs. Cardiomyocytes were treated with 3,3′,5-Triiodo-L-thyronine (T3, T5516, Sigma Aldrich) for 6 days at a concentration of 15 nM. NRCs were stimulated with exogenous ADP (A2754, Sigma Aldrich) at a concentration of 10 μM for 3 days. Oligomycin A (75351, Sigma Aldrich) and the AMPK inhibitor Compound C (CC; 171260, Sigma Aldrich) were applied at concentrations of 20 nM for 2 days and 5 μM for 24 h, respectively. NRC were randomly chosen for treatment the day after isolation.

### Isolation of Adult Mouse Cardiomyocytes

The left ventricle of adult mouse hearts was cut into pieces of 1 mm³ and fixed for 2 h with 4% paraformaldehyde (PFA)/PBS. The biopsies were digested with 1000 U collagenase type II (17101-015, Gibco) in HBSS for approximately 42 h at 37° C. while rotating. Appropriate concentrations of isolated adult cardiomyocytes were centrifuged at 600 rpm for 1 min onto gelatin-coated microscope slides using a cytospin (SCA-0030, Shandon Southern).

### Human Cardiomyocyte Culture

Human induced pluripotent stem cell-derived human cardiomyocytes (iPSC-CM) were purchased from Cellular Dynamics International (CMC-100-010-001) and cultured as recommended by the manufacturer. These cells show a clear sarcomeric organization as evidenced by striations. Furthermore, the cells are terminally differentiated as they do not proliferate and cannot be passaged. Gene expression analysis showed that iPSC-CM express more than 200 cardiac genes, among them α-actinin and cardiac troponin T, whose expression profiles are similar to adult human cardiac tissue. Cells started to beat spontaneously after 24-48 h post thawing and formed electrically connected syncytial layers that beat in synchrony. Cells were deemed suitable for studies when they formed a synchronically beating syncytium. Cells were randomly chosen for transduction with lentiviruses after 6-7 days in culture and experiments were performed on day 10.

### Isolation of Mitochondria

Mitochondria were isolated using the Mitochondria Isolation Kit for Cultured Cells (ab110171, Abcam). Briefly, 1.2×10⁶ NRCs were seeded per 6 cm dish and cells collected by scraping. After a freeze-thaw step to weaken the cell membrane, the cells were resuspended to a protein concentration of 5 mg/mL in Reagent A and incubated for 10 min on ice. The cells were homogenized with 30 strokes in a Dounce Homogenizer and centrifuged at 1000 g for 10 min at 4° C. The pellet was resuspended in the same volume of Reagent B than was used for Reagent A and rupturing and centrifugation was repeated. The supernatants after the two centrifugation steps were combined and centrifuged at 12000 g for 15 min at 4° C. Pellet was resuspended in 80 μL Reagent C supplemented with protease inhibitors and stored at −80° C. Mitochondrial ADP:ATP quantification was performed using the EnzyLight ADP/ATP Ratio Assay Kit (ELDT-100, Bioassay Systems) as recommended by the manufacturer.

### Lentivirus Production and Infection

HEK-293T cells were transfected at 80-90% confluence with polyethylenimine (PEI) transfection reagent. 10 μg transgene, 7.5 μg pMD2.G and 6.5 μg psPAX2 were mixed with 2 ml serum-free DMEM and 45 μg PEI per 10 cm dish. After 10 min incubation at room temperature (RT) the DNA/PEI-complexes were added slowly to the cells cultured in DMEM containing 0.5% FCS and L-glutamine. Medium was changed to NRC maintenance medium 4 h after transfection. Lentiviruses were harvested 48 h after transfection and stored at −80° C. NRC were infected 20 h after isolation and incubated at 37° C./5% C02 overnight.

### AAV9 Constructs

For the generation of the adeno-associated viral constructs, an shRNA targeting Mthfd1l was cloned into a pSico vector where the U6 promoter-driven shRNA expression is controlled in a Cre dependent manner. The shRNAs targeting Mthfd1l were designed using the online software pSico Oligomaker (MIT, version 1.5). The following shRNA sequence was used: AAV9-fl/fl-shMthfd1l,

Sense (SEQ ID NO 003):
TGAATGGTGTCAGAGAATTTTTCAAGAGAAAATTCTCTGACACCATTCT
TTTTTC,
antisense (SEQ ID NO 004):
TCGAGAAAAAAGAATGGTGTCAGAGAATTTTCTCTTGAAAAATTCTCTG
ACACCATTCA.

After in vitro validation of the knockdown efficiency of the shRNA using Adeno-Cre (Cat No ADV-005, Cell Biolabs) expression to induce Cre-mediated recombination and activation of shMthfd1l expression, the region bearing the U6 promoter, the shRNA and the TATA-lox sites of the pSico construct was amplified and ligated into Nhel/Xhol linearized AAV-bGH(+) vector. All other viruses were designed and engineered by Targeted Transgenesis. AAV9 viruses were injected at a concentration of 1.2×10¹³ genome copies (GC) per kg body weight into tail veins of 3-week-old Mlc2v-cre⁻ and Mlc2v-cre⁺ mice.

In Vitro Administration of Locked Nucleic Acids (LNA), miRNA Mimics and siRNAs

miRCURY LNA Power Inhibitors were added directly to the cell culture medium at a final concentration of 50 nM and fresh LNAs added every second day. The following miRCURY LNA Power Inhibitors were purchased from Exiqon: i-mmu-miR27b-5p (4101712-101). Negative Control A (199006-101) was used as non-targeting LNA.

miRIDIAN microRNA Mimics were transfected into NRCs using Lipofectamine 2000 (Invitrogen). Per well in a 96-well plate, 0.4 μL Lipofectamine 2000 was mixed with 25 μL OptiMEM (Invitrogen). After incubation of 5 min at RT, the mixture was added to an Eppendorf tube containing 6.25 nM or 12.5 nM miRNA mimics in 50 μL OptiMEM and incubated for 20 min at RT to form the complexes. 50 μL of the complexes was added to the cells cultured in 100 μL medium without antibiotics. The medium was replaced 4 h after transfection and cells were harvested 48 h after transfection. The following miRIDIAN microRNA Mimics were purchased from Dharmacon: mmu-miR27b-3p (C-310380-05-0005), mmu-miR27b-5p (C-310810-01-0005). miRIDIAN microRNA Mimic Negative Control #1 (CN-001000-01) was used as negative control.

siRNAs were transfected at a final concentration of 50 nM into NRCs using the DharmaFECT 1 transfection reagent (Dharmacon) as recommended by the manufacturer. Gene expression was analysed 72 h after transfection. The following ON-TARGETplus SMARTpool siRNAs from Dharmacon were used: Ccnd1 (L-089285-02-0005), Ccne1 (L-101575-02-0005) and Cdkn1b (L-090938-02-0005). Non-targeting siRNA pool (D-001810-10-05) was used as control.

### Transient Transfections

pCMV6 MTHFD1L (RC223034, Origene) or pEBG-AMPK₁₋₃₁₂ (#27632, addgene) were transiently transfected into primary neonatal rat cardiomyocytes 2 days after isolation using Lipofectamine 2000 (Invitrogen) according to the manufacturer's instructions. Cells were harvested and analyzed 48 h after transfection.

### Plasmid Constructions

The pLenti-HIF1αΔODD puro lentiviral expression vector was generated by subcloning the HIF1αΔODD fragment from a pcDNA3-HA-HIF1αΔODD (401-603) plasmid (Huang, L. E., et al., Proc Natl Acad Sci USA, 95, 7987-7992). into pLenti-pgk puro vector as described previously (Troilo, A., et al., 2014, EMBO Rep, 15, 77-85). As a corresponding control the pLenti-pgk puro empty vector was used. The miR27b overexpression construct was generated by amplification of the precursor mir27b sequence and the flanking sequence of 158 bp from either end of the mir27b precursor transcript from mouse genomic DNA. The sequence was cloned into the pLKO.1 CMV puro construct provided by A. Ittner (ETH Zurich), by using EcoRI and Sall restriction sites. As a control vector the empty pLKO.1 CMV puro construct was used.

Generation of miRNA Promoter-Luciferase Constructs

A 1.0 kilobyte (kb) fragment of the mir23b, mir24-1 and mir27b promoter was amplified from mouse genomic DNA and cloned into the pGL3 luciferase reporter vector (Stratagene) between the Xhol and HindIII restriction sites. Mutation of the HRE in the mir27b promoterwas generated by recombinant PCR (Elion, E. A., et al., 2007, Curr Protoc Mol Biol, Chapter 3, Unit 3 17). Sense and Antisense primers were designed bearing HRE mutations, which were used to amplify the mutant 5′ and 3′ regions of the mir27b promoter, respectively. Afterwards, the 5′ and 3′ products generated from the respective PCR reactions were mixed at a 1:1 ratio and the entire fragment was amplified using primers targeting the 5′ and 3′ ends of the promoters. The wildtype constructs and the mutation of the HRE in the mir27b promoter were confirmed by DNA sequencing (Microsynth).

### Generation of Atp5a1 3′ UTR Luciferase Construct

3′ UTR of Atp5a1 was amplified from mouse cDNA and cloned into the pmirGLO Dual-Luciferase miRNA Target Expression Vector (Promega). Mutation of mir27b binding site on the 3′ UTR of Atp5a1 was generated by recombinant PCR. Sense and antisense primers were generated bearing mir27b binding site mutations, which were used to amplify the mutant 5′ and 3′ regions of the 3′UTR, respectively. Afterwards, the 5′ and 3′ products generated from the respective PCR reactions were mixed at a 1:1 ratio and the entire sequence was amplified using primers targeting the 5′ and 3′ ends of the Atp5a1 3′UTR. The mutation of the mir27b binding site in the 3′ UTR of Atp5a1 was confirmed by sequencing (Microsynth).

shRNA Knockdown of Mthfd1l

shRNAs targeting Mthfd1l were designed using the online software BLOCK-iT RNAi Designer (Life Technologies) and compared against the rat genome using BLAST. The shRNA was cloned into the pLKO.1 vector. Sense and antisense oligomers were resuspended to a concentration of 20 μM in Annealing Buffer (100 mM NaCl, 50 mM HEPES, pH 7.4). 5 μL of sense and antisense oligomers were mixed and annealed by incubating them in a beaker containing boiling water and letting it cool to room temperature. The annealed oligomers were ligated into the pLKO.1 vector between the AgeI and EcoRI restriction sites. 50 ng vector, 200 ng annealed oligomers, 1 μL T4 DNA ligase were mixed with 1 μL 10× ligation buffer and brought to a final volume of 10 μL with PCR-grade water. The ligation reaction was incubated overnight at 16° C. and 2 μL of the ligation reaction was transformed into competent Stbl3 bacteria by heat shock transformation. Colonies were screened for insertion of shRNA by restriction digest using NdeI and BamHI and positive clones were confirmed by DNA sequencing (Microsynth).

### Lentiviral and Expression Constructs

For knockdowns with lentiviral shRNAs the following TRC pLKO.1 shRNA vectors were used: Atp5a1 (shAtp5a1, TRCN0000076239), Hif1α (shHif1α, TRCN0000232220) and Vhl (shVhl, TRCN0000436052). pLKO.1 vector containing non-silencing shRNA (nsRNA; SHC002, Sigma) was used as a control. pLenti ATP5A1 (RC214840L1) and pCMV6 MTHFD1L (RC223034) plasmids were from Origene. pEBG-AMPKα1(1-312) was a gift from Reuben Shaw (Addgene plasmid #27632).

RNA-Isolation, Reverse Transcription and qRT-PCR

Samples were harvested in Trizol (Invitrogen) and total RNA isolated as recommended by the manufacturer. 750 ng RNA were reverse transcribed into cDNA using RNA to cDNA EcoDry Premix (random hexamers) kit (Clontech, Cat No 639545) following the manufacturer's instructions. Quantitative real-time PCR (qRT-PCR) reactions were set up using iTaq Universal SYBR Green Supermix (Biorad, Cat No 1725121) according to manufacturer's recommendations and run on a PikoReal Real-Time PCR machine (Thermo Scientific). Ct values were normalized against the housekeeping gene Hprt1. The qRT-PCR primer sequences are shown in Table 6.

To assess mature miRNA levels, 10 ng total RNA was transcribed into cDNA using TaqMan MicroRNA Reverse Transcription Kit (4366596, Applied Biosystems) as recommended by the manufacturer. qRT-PCR was performed using TaqMan 2× Universal PCR Master Mix (4304437, Applied Biosystems) following the manufacturers instructions and run on a DNA Engine Opticon 2 (Bio-Rad). Ct values were normalized against the housekeeping genes snoRNA (rat) or snoRNA202 (mouse). The following primers from Thermo Fisher Scientific were used: miR23b-3p (ID000400), miR23b-5p (ID243680_mat) miR24-3p (ID000402), miR24-5p (ID000488), miR27b-3p (ID000409), miR27b-5p (ID002174), snoRNA (ID001718) and snoRNA202 (ID001232).

### PCR

To assess the integrity of the Nnt transcript in BL/6N and BL/6J mice, the PCR was performed using cardiac cDNA as described previously (Huang, T. T., et al., 2006, Hum Mol Genet, 15, 1187-1194).

### Chromatin Immunoprecipitation

ChIP assays were performed with material from NRCs and the assays carried out using the ChIP-IT Kit (Active Motif) according to the manufacturer's instructions and analyzed by qRT-PCR. ChIP was performed with a ChIP-grade antibody against Hif1α (mouse, ab1, Abcam). In silico promoter analyses and alignments were performed using MatInspector and DiAlignTF (Genomatix). Primer sequences used for mir27b in the ChIP were 5′-GCATGCTGATTTGTGACTTGAG-3′ (SEQ ID NO 006) and 5′-CCTCTGTTCTCCAAACTGCAG-3′ (SEQ ID NO 007).

### MicroRNA Microarray

The microRNA microarrays were performed on 3 biological replicates of Vhl cKO mice and three control mice (Vhl fl/fl), and on 3 biological replicates of Hif1α cKO mice subjected to TAC and three controls subjected to TAC surgery (TAC Hif1α fl/fl), respectively. Cardiac dimensions and function were confirmed in all mice by echocardiography. Total RNA was isolated from left ventricle and miRNAs were labelled using the miRCURY LNA microRNA Power Labelling Kit (Exiqon) and hybridized on miRNA arrays (miRXplore) that carry 1194 DNA oligonucleotides with the reverse-complementary sequence of the mature miRNAs. These arrays cover 728 human, 584 mouse, 426 rat and 122 viral miRNAs, each spotted on the arrays in quadruplicate. The Cy5-labelled miRNAs were normalized to a reference pool of miRNAs that were simultaneously labeled with Cy3. All the data are represented as ratios of logarithmic values between the diseased and control animals and deposited under GSE62418.

### NADPH Quantification

4×10⁵ NRCs were seeded per well in a 3 cm dish and the assay was performed using the NADP/NADPH Quantitation Kit (MAK038, Sigma) as recommended by the manufacturer. Data was normalized to protein amount in the cell lysate by the Bradford assay.

### ATP Quantification In Vivo

ATP was separated and quantified on an anion exchange column (Nucleosil 4000-7 PEI, 50/4 from Macherey-Nagel) with a linear gradient (0-1.5 M NaCl in 10 mM Tris-HCl, pH 8.0) using an HPLC system equipped with two independent UV-visible spectrometers (Shimadzu,). Elution of samples was monitored at 259 and 220 nm. The 220 nm wavelength was used to detect possible traces of contaminants.

### ADP and ATP Assays

For the ADP/ATP ratio analysis, 4×10⁵ NRCs were seeded per well in a 3 cm dish and the assay was performed using the EnzyLight ADP/ATP Ratio Assay Kit (ELDT-100, Bioassay Systems) as recommended by the manufacturer. For ADP and ATP quantifications the EnzyLight™ ADP Assay Kit (EADP-100, BioAssay Systems) and EnzyLight ATP Assay Kit (EATP-100, BioAssay Systems) were used as recommended by the manufacturers, respectively. The signal was measured on a FLUOstar Omega plate reader (BMG). For ADP/ATP quantification in human and mouse heart tissue, the assay was performed using the ADP/ATP Ratio Assay Kit (ab65313, Abcam) as recommended by the manufacturer with small modifications. Briefly, the heart tissue was frozen and stored in liquid nitrogen immediately after the harvest. The tissue was powdered with a mortar and suspended in lysis buffer (10 μl/mg of tissue powder) for 5 min at room temperature. After the centrifugation at 10000 g for 1 min, the supernatant was used for the assay. Data was normalized to protein amount in the supernatant by the Bradford assay.

### ATP Synthase Enzyme Activity Assay

4×10⁵ cells were seeded per 3 cm dish. Cells were harvested by trypsinization, followed by centrifugation at 1.2 krpm for 5 min. Pellet was washed 1× with PBS (Invitrogen) and centrifuged at 1.2 krpm for 5 min. Pellet was resuspended in 100 μl PBS and frozen at −80° C. and sample preparation was continued using the ATP synthase Enzyme Activity Assay Kit (ab109714, Abcam) as recommended by the manufacturer. Absorbance was measured at 340 nm and 30° C. for 3 h at 1 min intervals using a FLUOstar Omega plate reader (BMG Labtech). ATP synthase activity was normalized to protein concentration.

### Luciferase Assay

4×10⁴ NRCs were plated in a white 96-well plate and cultured for 3 days. 40 ng of wildtype or mutant pmirGLO Atp5a1 3′UTR construct was co-transfected with 6.25, 12.5 or 25 nM control or miR27b mimics using Lipofectamine 2000 (Invitrogen) as recommended by the manufacturer. Luciferase activity was measured 24 h after transfection using the Dual Luciferase Reporter Assay System (Promega) as recommended by the manufacturer on a FLUOstar Omega Microplate Reader (BMG Labtech).

To assess promoter activity, 20 ng pGL3 vector was co-transfected with 1.25 ng Renilla and 0-260 ng HIF1αΔODD. Luciferase activity was measured 36 h after transfection using the Dual Luciferase Reporter Assay System (Promega) as recommended by the manufacturer. E2F transactivation activities were measured using the Cignal Reporter Assay Kit (CCS-003L, Qiagen) according to the manufacturer's instructions using the Dual Luciferase ReporterAssay System (Promega).

### Immunoblotting

Heart tissue was solubilized in blue wonder sample buffer (3.7 M urea, 134.6 mM Tris pH 6.8, 5.4% (v/v) sodium dodecyl sulphate (SDS), 2.3% (v/v) NP-40, 4.45% (v/v) β-mercapto-ethanol, 4% (v/v) glycerol, 60 mg/L bromphenol blue) and proteins denatured for 5 min at 95° C. after homogenization using an Ultra-Turrax T10 tissue homogeniser (IKA). NRCs were washed twice with ice-cold PBS and harvested in blue wonder sample buffer. Samples were sonicated to reduce viscosity using a Bransonic 5510 Ultrasonic water bath (Branson) and boiled for 5 min. After brief centrifugation, protein lysates were loaded into 8% or 10% polyacrylamide minigels (Biorad) and transferred to nitrocellulose membrane (GE Healthcare) by wet transfer. Membranes were blocked in 5% (w/v) milk powder (Biorad) in TBST before incubation with primary antibody diluted in 5% (w/v) bovine serum albumin (BSA) in TBST for 2 hours at room temperature or overnight at 4° C. Following three washes in 5% (w/v) milk powder in TBST, membranes were incubated for 1-2 hours with the appropriate HRP conjugated secondary antibody (anti-Goat IgG HRP, 61-1620; anti-Mouse IgG HRP, 62-6520; anti-Rabbit IgG HRP, 65-6120, Invitrogen) at a dilution of 1:5000. Membranes were then washed three times with TBST before detection with ECL (Amersham) on X-ray RX NIF films (Fisher Scientific) to detect the chemiluminescence. Signal intensities were quantified by densitometry using Image J (version 1.47) (Schneider, C. A., et al., 2012, Nat Methods, 9, 671-675). The following antibodies were used for immunoblotting: anti-α-actinin (A7811, Sigma), anti-ACC (3676, Cell Signaling), anti-phospho-ACC Ser79 (11818, Cell Signaling), anti-AMPKα (5832, Cell Signaling), anti-phospho-AMPKα Thr172 (2535, Cell Signaling), anti-Atp5a1 (ab14748, Abcam), anti-cardiac actin (61075, Progen Biotechnik), anti-Hif1α (NB100-479, Novus Biologicals), methylated Lysine (di methyl) (ab23366, Abcam), anti-Mthfd1l (ab116615, Abcam), anti-Rb (9309, Cell Signaling), anti-phospho-Rb Ser807/811 (8516, Cell Signaling), anti-Vhl (2738, Cell Signaling) and IgG isotype-control antibody (ab171870).

### Co-Immunopprecipitation.

0.5×10⁷ transfected (pEBG-AMPK₁₋₃₁₂) or transduced (HIF1αΔODD-lentiviruses) NRC per condition were lysed in TNN cell lysis buffer (50 mM Tris-HCl pH 7.5, 250 mM NaCl, 5 mM EdTA, 0.5% NP-40.50 mM NaF, 0.5 mM EGTA, 1 mM PMSF, 1 mMDTT, and protease inhibitor cocktail) for 30 min. Magnetic dynabeads (Invitrogen) were incubated with anti-AMPKα antibody or IgG control antibody (ab171870) for 10 min at room temperature. AMPK was then immunoprecipitated from cell lysate by incubation for 3 hours at 4° C., IgG control antibody (ab171870). Finally, reaction was terminated by boiling beads in 6× Laemmli buffer for 5 min. Samples were resolved in 10% SDS-PAGE, and Western blot analysis was performed with phospho-RbSer800/804 (Cell Signaling Technology) and phospho-AMPKα Thr172 (Cell Signaling).

### Immunofluorescent Stainings

Immunofluorescent stainings were performed as described previously (Krishnan, J., et al., 2009, Cell Metab, 9, 512-524). After fixation of NRCs with 4% PFA/PBS, the cells were permeabilized and incubated with primary antibodies diluted in 2% (v/v) HS for 1h at RT. After 3 washes with PBS for 5 min, cells were incubated with 4′,6-diamidino-2-phenylindole (DAPI; D1306, Thermo Fisher Scientific, 0.1 μg/ml), Phalloidin 555 (A34055, Molecular Probes), AlexaFluor 647 anti-mouse (A-11001, Thermo Fisher Scientific) and AlexaFluor 488 anti-mouse (A-21235, Thermo Fisher Scientific) secondary antibody for 1 h at room temperature. For adult cardiomyocytes, the slides were incubated with the primary and secondary antibodies overnight at 4° C. in a humidified chamber. Slides and dishes were mounted onto glass slides (Fisher Scientific) with a drop of ProLong Antifade (Thermo Fisher Scientific). The slides/dishes were fixed with clear nail polish and left to dry. The following primary antibodies were used: sarcomeric α-actinin (A7811, Sigma Aldrich), Ki67 (ab15580, Abcam) and phospho-Histone H3 (Ser10) (05-817, Cell Signaling). Fluorescent images were acquired with the SP5 confocal microscopy (Leica) using a 20× magnification. Cell size was quantified blinded using the software Image J (version 1.47).

### Immunohistochemistry

Hearts were embedded in optimal cutting temperature (OCT) compound and sectioned at 10 μm. Sections were fixed for 10 min with 4% PFA/PBS and after 2 washes with PBS for 2 min blocked for 1 h with 2% HS/PBS for 1 h at room temperature. After permeabilisation for 10 min with 0.2% Triton X-100/PBS, the sections were washed 3 times with PBS for 5 min and incubated with primary antibodies against sarcomeric α-actinin (A7811, Sigma Aldrich, 1:800) and Laminin (ab11575, Abcam, 1:300) diluted in 2% (v/v) HS overnight in a humidified chamber at 4° C. After 3 washes with PBS for 10 min, sections were incubated with 4′,6-diamidino-2-phenylindole (DAPI; D1306, Thermo Fisher Scientific, 0.1 μg/ml), AlexaFluor 555 anti-mouse (A-21422, Thermo Fisher Scientific, 1:500) and AlexaFluor 488 anti-rabbit (A-11034, Thermo Fisher Scientific, 1:500) secondary antibody for 2 h at room temperature. Sections mounted with a drop of ProLong Antifade (Thermo Fisher Scientific) and fixed with clear nail polish and left to dry. Fluorescent images were acquired with the SP8 confocal microscopy (Leica) using a 20× magnification. The entire z-axis was imaged and z-stack image generated from approximately 0.2 μm steps. To determine the number of nuclei per myocyte the inventors included only myocytes that had been cut along their longitudinal axis. Nuclei that were surrounded by the extracellular matrix stain laminin were excluded from the analysis. Quantification of nuclei was performed blinded.

Cryosections were stained with hematoxylin and eosin (H&E) or picrosirius red (Sigma). Slides were visualized using a Motic AE2000 with a Moticam 3 or the Axioscan.Z1 slide scanner (Carl Zeiss).

### [³H]Leucine Incorporation Assay

[³H]leucine incorporation assay was used to measure de novo protein synthesis as an indirect readout for cell growth (Fukuzawa, J., et al., 2000, Hypertension, 35, 1191-1196). 4×10⁵ cells were seeded per 3 cm dish. After 3 days, cells were serum-starved overnight. Next day, cells were cultured in leucine-free medium for 4 h, followed by culturing in maintenance medium containing labeled L-[4,5-³H(N)]isoleucine (specific activity 30-60 μCi/mmol, ART0233, American Radiolabelled Chemicals) at a concentration of 0.5 μCi/mL for 20 h. The next day, cells were washed with PBS, trypsinized and the radioactivity was measured for 5 min in in the liquid scintillation analyzer Tri-Carb 2800TR (Perkin Elmer). Cells were counted blinded with a Neubauer chamber after trypsinization and scintillation counts were normalized to absolute cell number.

### Nucleic Acid Incorporation Assays

4×10⁵ cells were seeded per 3 cm dish. After 4 days, cells were starved of glucose for 1.5 h and serine or glycine for 3 h. For radiolabelled glucose incorporation, cells were incubated for 4 h in glucose-free DMEM (Invitrogen) containing 1 μCi/mL uniformly labeled [U-¹⁴C]glucose (specific activity 250-360 mCi/mmol, NEC042V250UC, Perkin Elmer). For radiolabelled serine incorporation, cardiomyocytes were incubated overnight in MEM (Invitrogen) supplemented with MEM Vitamin Solution (Invitrogen) and containing 0.6 μCi/mL radiolabelled serine at carbon 3 ([3-¹⁴C]serine, specific activity 50-62 mCi/mmol, NEC827050UC, Perkin Elmer). For radiolabelled glycine incorporation, cells were incubated in BME (Invitrogen) containing 1 μCi/mL uniformly radiolabelled glycine ([¹⁴C(U)]glycine, specific activity >100 mCi/mmol, NEC276E250UC, Perkin Elmer) for 4 h. After washing the cells three times with PBS, cells were harvested in Trizol (Invitrogen) and nucleic acids isolated as recommended by the manufacturer. Isolated nucleic acids were transferred to a scintillation vial containing 4 mL of the liquid scintillation cocktail Ultima Gold (Perkin Elmer) and radioactivity was measured for 5 min in the Liquid Scintillation Analyzer Tri-Carb 2800TR (Perkin Elmer). Scintillation counts were normalized to absolute nucleic acid quantity.

### Fatty Acid Oxidation

Fatty acid oxidation rate in animal tissues using [1-¹⁴C]-palmitic acid (NEC075H, Perkin Elmer) was determined as described previously (Huynh, F. K., et al., 2014, Methods Enzymol, 542, 391-405). with the exception that 0.7% BSA/500 μM palmitate/2 μCi ¹⁴C-palmitate was used per reaction. Radioactivity was measured for 5 min in the Liquid Scintillation Analyzer Tri-Carb 2800TR (Perkin Elmer). Scintillation counts were normalized to protein amount.

### Flow Cytometry

1×10⁶ NRCs were plated on a 6 cm dish (Nunc) and after 4-6 days cells were harvested by trypsinization. Cells were washed 1× with PBS and centrifuged at 80 g for 5 min. Cells were resuspended in 500 μL PBS and 5 mL cold 70% (v/v) ethanol (kept at −20° C.) was added immediately. The fixed cells were kept at 4° C. for up to 1 week. Prior to flow cytometry analysis, cells were centrifuged and washed with PBS. After centrifugation at 80 g for 5 min the cells were resuspended in 500 μL propidium iodide solution (69 μM propidium iodide in 38 mM sodium citrate, pH 7.4) containing 40 μg/mL RNase and incubated for 1 h at 37° C. Samples were run on the ImageStream (Amnis), a flow cytometer coupled to fluorescence image acquisition to obtain representative images of the flow cytometry data. The multinucleation was quantified from the ImageStream images.

### Formate Assay

4×10⁵ cells were seeded per 3 cm dish and the assay was performed using the Formate Assay Kit (Sigma). Cells were washed twice with cold PBS and collected in 25 μL Formate Assay Buffer by scraping. After centrifugation at 15000 g at 4° C. for 5 min, 20 μL lysate was mixed with 5 μL Formate Assay Buffer and added to a 96-well plate. 25 μL of the Reaction Mix consisting of 23 μL Formate Assay Buffer, 1 μL Formate Enzyme Mix and 1 μL Formate Substrate Mix was added to the plate containing the lysate. The samples were incubated for 1 h at 37° C. protected from light and the absorbance was measured at 450 nm on a FLUOstar Omega plate reader (BMG Labtech). The data was normalized to protein concentration.

### Metabolomics

4×10⁵ NRCs were cultured per 3 cm dish. The whole cell culture plates were snap frozen in liquid nitrogen after the cells were washed with 75 mM Ammonium carbonate (Sigma), adjusted to pH 7.4 with acetic acid. The metabolites were extracted with cold extraction buffer (−20° C.) containing acetonitrile:methanol:water in a 40:40:20 ratio. Untargeted analysis of metabolites by flow injection-time-of-flight mass spectrometry as previously described (Fuhrer, T., et al., 2011, Anal Chem, 83, 7074-7080). Data was processed and analyzed with Matlab. Metabolomics analysis performed by Metabolon (FIG. 12d) was performed as previously described (Cimen, I., et al., 2016, Sci Trans/Med, 8, 358ra126).

### Lipidomics

Mouse heart samples were homogenized on ice in ammonium-bicarbonate buffer (150 mM ammonium bicarbonate, pH 7) with ultra-turax homogenizer. Protein content was assessed using BCA Protein Assay Kit (Thermo Fisher). Equivalents of 20 μg of protein were taken for mass spectrometry analysis. Mass spectrometry-based lipid analysis was performed by Lipotype GmbH (Dresden, Germany) as described (Sampaio, J. L., et al., 2011, Proc Natl Acad Sci USA, 108, 1903-1907). Lipids were extracted using a two-step chloroform/methanol procedure (Ejsing, C. S., et al., 2009, Proc Natl Acad Sci USA, 106, 2136-2141). Samples were spiked with internal lipid standard mixture containing: cardiolipin 16:1/15:0/15:0/15:0 (CL), ceramide 18:1;2/17:0 (Cer), hexosylceramide 18:1;2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1;2/12:0;0 (SM), cholesterol D6 (Chol) (all Avanti Polar Lipids), triacylglycerol 17:0/17:0/17:0 (TAG) and diacylglycerol 17:0/17:0 (DAG) (both Larodan Fine Chemicals). After extraction, the organic phase was transferred to an infusion plate and dried in a speed vacuum concentrator. 1st step dry extract was re-suspended in 7.5 mM ammonium acetate (Sigma) in chloroform/methanol/propanol (1:2:4, V:V:V) and 2nd step dry extract in 33% ethanol solution of methylamine in chloroform/methanol (0.003:5:1; V:V:V) (all liquids were LC grade obtained from VWR). All liquid handling steps were performed using Hamilton Robotics STARlet robotic platform with the Anti Droplet Control feature for organic solvents pipetting.

Samples were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MSMS was triggered by an inclusion list encompassing corresponding MS mass ranges scanned in 1 Da increments ¹¹⁹ Both MS and MSMS data were combined to monitor CE, DAG and TAG ions as ammonium adducts; PC, PC O−, as acetate adducts; and CL, PA, PE, PE O−, PG, PI and PS as deprotonated anions. MS only was used to monitor LPA, LPE, LPE O−, LPI and LPS as deprotonated anions; Cer, HexCer, SM, LPC and LPC O− as acetate adduct and cholesterol as ammonium adduct of an acetylated derivative.

Data were analyzed with a lipid identification software based on LipidXplorer (Herzog, R., et al., 2012, PLoS One, 7, e29851) (Herzog, R., et al., 2011, Genome Biol, 12, R8). Data post-processing and normalization were performed using an in-house developed data management system. Only lipid identifications with a signal-to-noise ratio >5, and a signal intensity 5-fold higher than in corresponding blank samples were considered for further data analysis.

All downstream analyses were performed in R¹²³on the mol %-transformed dataset, i.e., after transforming raw data (picomol) to mole percent (each quantity was divided by the sum of the lipids detected in its respective sample and multiplied by 100). Principal Component Analysis (PCA) was computed using the Singular Value Decomposition function. Total Carbon chain length and saturation plots result from grouping together all the lipids that present the same number of carbon atoms (total length) or the same number of double bonds (saturation) and calculating their mean and standard deviation in each cohort of samples. The difference between the means was calculated for each species by subtracting the mean of the controls from the mean of the treated samples (Treated minus Reference), but only the lipids with a delta exceeding 10.31 are shown in the supplemental material. Significance was calculated by means of the non-parametric test Wilcoxon and, in case of multiple comparisons, p-values were adjusted after the Benjamini-Hochberg correction. Significance was set at p<0.05. Alongside R-base functions the following packages were used: factoextra (Kassambara, A. & Mundt, F., 2017, factoextra: Extract and Visualize the Results of Multivariate Data Analyses.)., reshape2 (Wickham, H., 2017, Journal of Statistical Software, 21, 1-20). and ggplot2 (Wickham, H., 2009, ggplot2: Elegant Graphics for Data Analysis).

### Bioinformatic Analysis

In silico promoter analyses were performed using MatInspector (Genomatix). Sequence alignments were performed with BLAST alignment (http://www.ncbi.nlm.nih.gov/blast). shRNAs were designed by proprietary algorithm (Targeted Transgenesis), or computationally predicted using pSico Oligomaker (MIT, version 1.5), BLOCK-iT RNAi Designer (Life Technologies). To predict miR27b targets the miRWalk 2.0 database (http://mirwalk.uni-hd.de) which includes many databases such as TargetScan, miRanda and RNA22 was used (Dweep, et al., 2011, J Biomed Inform, 44, 839-847). Confocal images were quantified using Image J (version 1.47) (Schneider, C. A., et al., 2012, Nat Methods 9, 671-675).

1. A method for treatment or prevention of heart disease comprising:
administering to a subject an oligonucleic acid agent directed at and capable of specifically inhibiting and/or degrading the microRNA miR27b-5p, thereby treating or preventing the heart disease.

2. The method of claim 1, wherein the oligonucleic acid agent comprises, or essentially consists of a hybridizing sequence of nucleotides, which is capable of forming a hybrid with the microRNA miR27b-5p.

3. The method of claim 1, wherein the oligonucleic acid agent comprises, or essentially consists of, the sequence ACC AAT CAG CTA AGC T (SEQ ID NO 001).

4. The method of claim 1, wherein the oligonucleic acid agent is an antisense oligonucleotide.

5. The method of claim 1, wherein the oligonucleic acid agent comprises one or several, or essentially consists of, locked nucleic acid (LNA) and/or peptide nucleic acid (PNA) moieties, particularly wherein the oligonucleic acid agent essentially consists of locked nucleic acid (LNA) moieties.

6. The method of claim 5, wherein the LNA moieties are connected by thiophosphate bonds.

7. The method of claim 1, wherein the oligonucleic acid agent comprises 12-20 nucleotides, particularly 14-16 nucleotides.

8. The method of claim 1, wherein the oligonucleic acid agent comprises or essentially consists of a central block of 5 to 10 deoxyribonucleotides flanked on either side by 2′-O modified ribonucleotides or PNA oligomers, more particularly a central block of 5 to 10 deoxyribonucleosides flanked by LNA nucleoside analogues, even more particularly wherein said LNA nucleoside analogues are linked by phosphothioate moieties.

9. The method of claim 1, wherein the oligonucleic acid agent comprises or essentially consists of the sequence ACCA-atcagcta-AGCT (SEQ ID NO 005), wherein
the capital letters signify nucleoside analogues, particularly LNA, more particularly LNA linked by phosphothioate esters, and
the lower case letters signify DNA nucleosides linked by phosphate esters,
and the link between a nucleoside analogue and a DNA nucleoside is selected from phosphate ester and thiophosphate.

10. The method of claim 1, wherein the oligonucleic acid agent is linked to a nanoparticle, or encapsulated in a virus or a lipid complex.

11. The method of claim 1, wherein the heart disease is selected from cardiomyopathy, hypertrophic cardiomyopathy, aortic stenosis, hypertension and heart failure.

12. The method of claim 1, wherein the heart disease is associated with reduced or absent mitochondrial Complex V (ATP Synthase) activity.

13. (canceled)


SEQUENCE LISTING (8 sequences)

SEQ ID NO: 1 - DNA, 16 bp, Artificial Sequence
  oligonucleic acid agent
accaatcagctaagct

SEQ ID NO: 2 - RNA, 21 nt, Homo sapiens
gcagaacuuagccacugugaa

SEQ ID NO: 3 - DNA, 55 bp, Artificial Sequence
  oligonucleic acid agent
tgaatggtgtcagagaatttttcaagagaaaattctctgacaccattcttttttc

SEQ ID NO: 4 - DNA, 59 bp, Artificial Sequence
  oligonucleic acid agent
tcgagaaaaaagaatggtgtcagagaattttctcttgaaaaattctctgacaccattca

SEQ ID NO: 5 - DNA, 16 bp, Artificial Sequence
  oligonucleic acid agent
accaatcagctaagct

SEQ ID NO: 6 - DNA, 22 bp, Artificial Sequence
  Primer
gcatgctgatttgtgacttgag

SEQ ID NO: 7 - DNA, 21 bp, Artificial Sequence
  Primer
cctctgttctccaaactgcag

SEQ ID NO: 8 - DNA, 16 bp, Artificial Sequence
  oligonucleic acid agent
accaatcagctaagct